SEQUENCE LISTING (191 sequences)

SEQ ID NO: 1 - protein, 20 aa, Human herpesvirus 8
MDGTDALEKLTKGLSGGGGS

SEQ ID NO: 2 - protein, 20 aa, Human herpesvirus 8
ALEKLTKGLSGGGGSLHQTK

SEQ ID NO: 3 - protein, 20 aa, Human herpesvirus 8
TKGLSGGGGSLHQTKLLMEF

SEQ ID NO: 4 - protein, 20 aa, Human herpesvirus 8
GGGGSLHQTKLLMEFQLRGL

SEQ ID NO: 5 - protein, 20 aa, Human herpesvirus 8
LHQTKLLMEFQLRGLPVPAL

SEQ ID NO: 6 - protein, 20 aa, Human herpesvirus 8
LLMEFQLRGLPVPALLNSST

SEQ ID NO: 7 - protein, 20 aa, Human herpesvirus 8
QLRGLPVPALLNSSTTEQFL

SEQ ID NO: 8 - protein, 20 aa, Human herpesvirus 8
PVPALLNSSTTEQFLNTVAQ

SEQ ID NO: 9 - protein, 20 aa, Human herpesvirus 8
LNSSTTEQFLNTVAQLPTDL

SEQ ID NO: 10 - protein, 20 aa, Human herpesvirus 8
TEQFLNTVAQLPTDLSKFIR

SEQ ID NO: 11 - protein, 20 aa, Human herpesvirus 8
NTVAQLPTDLSKFIRDYRVF

SEQ ID NO: 12 - protein, 20 aa, Human herpesvirus 8
LPTDLSKFIRDYRVFALVRA

SEQ ID NO: 13 - protein, 20 aa, Human herpesvirus 8
SKFIRDYRVFALVRAAYFLE

SEQ ID NO: 14 - protein, 20 aa, Human herpesvirus 8
DYRVFALVRAAYFLEPPSSI

SEQ ID NO: 15 - protein, 20 aa, Human herpesvirus 8
ALVRAAYFLEPPSSIDPLEA

SEQ ID NO: 16 - protein, 20 aa, Human herpesvirus 8
AYFLEPPSSIDPLEAARALG

SEQ ID NO: 17 - protein, 20 aa, Human herpesvirus 8
PPSSIDPLEAARALGRLVDI

SEQ ID NO: 18 - protein, 20 aa, Human herpesvirus 8
DPLEAARALGRLVDILSSQP

SEQ ID NO: 19 - protein, 20 aa, Human herpesvirus 8
ARALGRLVDILSSQPPQNTA

SEQ ID NO: 20 - protein, 20 aa, Human herpesvirus 8
RLVDILSSQPPQNTAPAQPP

SEQ ID NO: 21 - protein, 20 aa, Human herpesvirus 8
LSSQPPQNTAPAQPPTSDDT

SEQ ID NO: 22 - protein, 20 aa, Human herpesvirus 8
PQNTAPAQPPTSDDTLNNCT

SEQ ID NO: 23 - protein, 20 aa, Human herpesvirus 8
PAQPPTSDDTLNNCTLLKLL

SEQ ID NO: 24 - protein, 20 aa, Human herpesvirus 8
TSDDTLNNCTLLKLLAHYAD

SEQ ID NO: 25 - protein, 20 aa, Human herpesvirus 8
LNNCTLLKLLAHYADQIAGF

SEQ ID NO: 26 - protein, 20 aa, Human herpesvirus 8
LLKLLAHYADQIAGFKTPAL

SEQ ID NO: 27 - protein, 20 aa, Human herpesvirus 8
AHYADQIAGFKTPALPPVPP

SEQ ID NO: 28 - protein, 20 aa, Human herpesvirus 8
QIAGFKTPALPPVPPGIIGL

SEQ ID NO: 29 - protein, 20 aa, Human herpesvirus 8
KTPALPPVPPGIIGLFTCVE

SEQ ID NO: 30 - protein, 20 aa, Human herpesvirus 8
PPVPPGIIGLFTCVEQMYHA

SEQ ID NO: 31 - protein, 20 aa, Human herpesvirus 8
GIIGLFTCVEQMYHACFQKY

SEQ ID NO: 32 - protein, 20 aa, Human herpesvirus 8
FTCVEQMYHACFQKYWAAAL

SEQ ID NO: 33 - protein, 20 aa, Human herpesvirus 8
QMYHACFQKYWAAALPPMWI

SEQ ID NO: 34 - protein, 20 aa, Human herpesvirus 8
CFQKYWAAALPPMWILTYDP

SEQ ID NO: 35 - protein, 20 aa, Human herpesvirus 8
WAAALPPMWILTYDPPTSPL

SEQ ID NO: 36 - protein, 20 aa, Human herpesvirus 8
PPMWILTYDPPTSPLQDWLI

SEQ ID NO: 37 - protein, 20 aa, Human herpesvirus 8
LTYDPPTSPLQDWLIVAYGN

SEQ ID NO: 38 - protein, 20 aa, Human herpesvirus 8
PTSPLQDWLIVAYGNKEGLL

SEQ ID NO: 39 - protein, 20 aa, Human herpesvirus 8
QDWLIVAYGNKEGLLLPSGI

SEQ ID NO: 40 - protein, 20 aa, Human herpesvirus 8
VAYGNKEGLLLPSGIPSEEV

SEQ ID NO: 41 - protein, 20 aa, Human herpesvirus 8
KEGLLLPSGIPSEEVLAKTL

SEQ ID NO: 42 - protein, 20 aa, Human herpesvirus 8
LPSGIPSEEVLAKTLVTEHH

SEQ ID NO: 43 - protein, 20 aa, Human herpesvirus 8
PSEEVLAKTLVTEHHELFVS

SEQ ID NO: 44 - protein, 20 aa, Human herpesvirus 8
LAKTLVTEHHELFVSRSNST

SEQ ID NO: 45 - protein, 20 aa, Human herpesvirus 8
VTEHHELFVSRSNSTETAVT

SEQ ID NO: 46 - protein, 20 aa, Human herpesvirus 8
ELFVSRSNSTETAVTMPVSK

SEQ ID NO: 47 - protein, 20 aa, Human herpesvirus 8
RSNSTETAVTMPVSKERALA

SEQ ID NO: 48 - protein, 20 aa, Human herpesvirus 8
ETAVTMPVSKERALAIYRVF

SEQ ID NO: 49 - protein, 20 aa, Human herpesvirus 8
MPVSKERALAIYRVFAKGEV

SEQ ID NO: 50 - protein, 20 aa, Human herpesvirus 8
ERALAIYRVFAKGEVVAENT

SEQ ID NO: 51 - protein, 20 aa, Human herpesvirus 8
IYRVFAKGEVVAENTPILAF

SEQ ID NO: 52 - protein, 20 aa, Human herpesvirus 8
AKGEVVAENTPILAFTDVEL

SEQ ID NO: 53 - protein, 20 aa, Human herpesvirus 8
VAENTPILAFTDVELSTLKP

SEQ ID NO: 54 - protein, 20 aa, Human herpesvirus 8
PILAFTDVELSTLKPHYLFI

SEQ ID NO: 55 - protein, 20 aa, Human herpesvirus 8
TDVELSTLKPHYLFIYDFII

SEQ ID NO: 56 - protein, 20 aa, Human herpesvirus 8
STLKPHYLFIYDFIIEALCK

SEQ ID NO: 57 - protein, 20 aa, Human herpesvirus 8
HYLFIYDFIIEALCKSYTYS

SEQ ID NO: 58 - protein, 20 aa, Human herpesvirus 8
YDFIIEALCKSYTYSCTQAR

SEQ ID NO: 59 - protein, 20 aa, Human herpesvirus 8
EALCKSYTYSCTQARLESFL

SEQ ID NO: 60 - protein, 20 aa, Human herpesvirus 8
SYTYSCTQARLESFLSRGID

SEQ ID NO: 61 - protein, 20 aa, Human herpesvirus 8
CTQARLESFLSRGIDFMTDL

SEQ ID NO: 62 - protein, 20 aa, Human herpesvirus 8
LESFLSRGIDFMTDLGQYLD

SEQ ID NO: 63 - protein, 20 aa, Human herpesvirus 8
SRGIDFMTDLGQYLDTATSG

SEQ ID NO: 64 - protein, 20 aa, Human herpesvirus 8
FMTDLGQYLDTATSGKQQLT

SEQ ID NO: 65 - protein, 20 aa, Human herpesvirus 8
GQYLDTATSGKQQLTHSQIK

SEQ ID NO: 66 - protein, 20 aa, Human herpesvirus 8
TATSGKQQLTHSQIKEIKYR

SEQ ID NO: 67 - protein, 20 aa, Human herpesvirus 8
KQQLTHSQIKEIKYRLLSCG

SEQ ID NO: 68 - protein, 20 aa, Human herpesvirus 8
HSQIKEIKYRLLSCGLSASA

SEQ ID NO: 69 - protein, 20 aa, Human herpesvirus 8
EIKYRLLSCGLSASACDVFR

SEQ ID NO: 70 - protein, 20 aa, Human herpesvirus 8
LLSCGLSASACDVFRTVIMT

SEQ ID NO: 71 - protein, 20 aa, Human herpesvirus 8
LSASACDVFRTVIMTLPYRP

SEQ ID NO: 72 - protein, 20 aa, Human herpesvirus 8
CDVFRTVIMTLPYRPTPNLA

SEQ ID NO: 73 - protein, 20 aa, Human herpesvirus 8
TVIMTLPYRPTPNLANLSTF

SEQ ID NO: 74 - protein, 20 aa, Human herpesvirus 8
LPYRPTPNLANLSTFMGMVH

SEQ ID NO: 75 - protein, 20 aa, Human herpesvirus 8
TPNLANLSTFMGMVHQLTMF

SEQ ID NO: 76 - protein, 20 aa, Human herpesvirus 8
NLSTFMGMVHQLTMFGHYFY

SEQ ID NO: 77 - protein, 20 aa, Human herpesvirus 8
MGMVHQLTMFGHYFYRCLGS

SEQ ID NO: 78 - protein, 20 aa, Human herpesvirus 8
QLTMFGHYFYRCLGSYSPTG

SEQ ID NO: 79 - protein, 20 aa, Human herpesvirus 8
GHYFYRCLGSYSPTGLAFTE

SEQ ID NO: 80 - protein, 20 aa, Human herpesvirus 8
RCLGSYSPTGLAFTELQKIL

SEQ ID NO: 81 - protein, 20 aa, Human herpesvirus 8
YSPTGLAFTELQKILTRASA

SEQ ID NO: 82 - protein, 20 aa, Human herpesvirus 8
LAFTELQKILTRASAEQTER

SEQ ID NO: 83 - protein, 20 aa, Human herpesvirus 8
LQKILTRASAEQTERNPWRH

SEQ ID NO: 84 - protein, 20 aa, Human herpesvirus 8
TRASAEQTERNPWRHPGISD

SEQ ID NO: 85 - protein, 20 aa, Human herpesvirus 8
EQTERNPWRHPGISDIPLRW

SEQ ID NO: 86 - protein, 20 aa, Human herpesvirus 8
NPWRHPGISDIPLRWKISRA

SEQ ID NO: 87 - protein, 20 aa, Human herpesvirus 8
PGISDIPLRWKISRALAFFV

SEQ ID NO: 88 - protein, 20 aa, Human herpesvirus 8
IPLRWKISRALAFFVPPAPI

SEQ ID NO: 89 - protein, 20 aa, Human herpesvirus 8
KISRALAFFVPPAPINTLQR

SEQ ID NO: 90 - protein, 20 aa, Human herpesvirus 8
LAFFVPPAPINTLQRVYAAL

SEQ ID NO: 91 - protein, 20 aa, Human herpesvirus 8
PPAPINTLQRVYAALPSQLM

SEQ ID NO: 92 - protein, 20 aa, Human herpesvirus 8
NTLQRVYAALPSQLMRAIFE

SEQ ID NO: 93 - protein, 20 aa, Human herpesvirus 8
VYAALPSQLMRAIFEISVKT

SEQ ID NO: 94 - protein, 20 aa, Human herpesvirus 8
PSQLMRAIFEISVKTTWGGA

SEQ ID NO: 95 - protein, 20 aa, Human herpesvirus 8
RAIFEISVKTTWGGAVPANL

SEQ ID NO: 96 - protein, 20 aa, Human herpesvirus 8
ISVKTTWGGAVPANLARDID

SEQ ID NO: 97 - protein, 20 aa, Human herpesvirus 8
TWGGAVPANLARDIDTGPNT

SEQ ID NO: 98 - protein, 20 aa, Human herpesvirus 8
VPANLARDIDTGPNTQHISS

SEQ ID NO: 99 - protein, 20 aa, Human herpesvirus 8
ARDIDTGPNTQHISSTPPPT

SEQ ID NO: 100 - protein, 20 aa, Human herpesvirus 8
TGPNTQHISSTPPPTLKDVE

SEQ ID NO: 101 - protein, 20 aa, Human herpesvirus 8
QHISSTPPPTLKDVETYCQG

SEQ ID NO: 102 - protein, 20 aa, Human herpesvirus 8
TPPPTLKDVETYCQGLRVGD

SEQ ID NO: 103 - protein, 20 aa, Human herpesvirus 8
LKDVETYCQGLRVGDTEYDE

SEQ ID NO: 104 - protein, 20 aa, Human herpesvirus 8
TYCQGLRVGDTEYDEDIVRS

SEQ ID NO: 105 - protein, 20 aa, Human herpesvirus 8
LRVGDTEYDEDIVRSPLFAD

SEQ ID NO: 106 - protein, 20 aa, Human herpesvirus 8
TEYDEDIVRSPLFADAFTKS

SEQ ID NO: 107 - protein, 20 aa, Human herpesvirus 8
DIVRSPLFADAFTKSHLLPI

SEQ ID NO: 108 - protein, 20 aa, Human herpesvirus 8
PLFADAFTKSHLLPILREVL

SEQ ID NO: 109 - protein, 20 aa, Human herpesvirus 8
AFTKSHLLPILREVLENRLQ

SEQ ID NO: 110 - protein, 20 aa, Human herpesvirus 8
HLLPILREVLENRLQKNRAL

SEQ ID NO: 111 - protein, 20 aa, Human herpesvirus 8
LREVLENRLQKNRALFQIRW

SEQ ID NO: 112 - protein, 20 aa, Human herpesvirus 8
ENRLQKNRALFQIRWLIIFA

SEQ ID NO: 113 - protein, 20 aa, Human herpesvirus 8
KNRALFQIRWLIIFAAEAAT

SEQ ID NO: 114 - protein, 20 aa, Human herpesvirus 8
FQIRWLIIFAAEAATGLIPA

SEQ ID NO: 115 - protein, 20 aa, Human herpesvirus 8
LIIFAAEAATGLIPARRPLA

SEQ ID NO: 116 - protein, 20 aa, Human herpesvirus 8
AEAATGLIPARRPLARAYFH

SEQ ID NO: 117 - protein, 20 aa, Human herpesvirus 8
GLIPARRPLARAYFHIMDIL

SEQ ID NO: 118 - protein, 20 aa, Human herpesvirus 8
RRPLARAYFHIMDILEERHS

SEQ ID NO: 119 - protein, 20 aa, Human herpesvirus 8
RAYFHIMDILEERHSQDALY

SEQ ID NO: 120 - protein, 20 aa, Human herpesvirus 8
IMDILEERHSQDALYNLLDC

SEQ ID NO: 121 - protein, 20 aa, Human herpesvirus 8
EERHSQDALYNLLDCIQELF

SEQ ID NO: 122 - protein, 20 aa, Human herpesvirus 8
QDALYNLLDCIQELFTHIRQ

SEQ ID NO: 123 - protein, 20 aa, Human herpesvirus 8
NLLDCIQELFTHIRQAVPDA

SEQ ID NO: 124 - protein, 20 aa, Human herpesvirus 8
IQELFTHIRQAVPDAQCPHA

SEQ ID NO: 125 - protein, 20 aa, Human herpesvirus 8
THIRQAVPDAQCPHAFLQSL

SEQ ID NO: 126 - protein, 20 aa, Human herpesvirus 8
AVPDAQCPHAFLQSLFVFQF

SEQ ID NO: 127 - protein, 20 aa, Human herpesvirus 8
QCPHAFLQSLFVFQFRPFVL

SEQ ID NO: 128 - protein, 20 aa, Human herpesvirus 8
FLQSLFVFQFRPFVLKHQQG

SEQ ID NO: 129 - protein, 20 aa, Human herpesvirus 8
FVFQFRPFVLKHQQGVTLFL

SEQ ID NO: 130 - protein, 20 aa, Human herpesvirus 8
RPFVLKHQQGVTLFLDGLQT

SEQ ID NO: 131 - protein, 20 aa, Human herpesvirus 8
KHQQGVTLFLDGLQTSLPPV

SEQ ID NO: 132 - protein, 20 aa, Human herpesvirus 8
VTLFLDGLQTSLPPVISLAN

SEQ ID NO: 133 - protein, 20 aa, Human herpesvirus 8
DGLQTSLPPVISLANLGDKL

SEQ ID NO: 134 - protein, 20 aa, Human herpesvirus 8
SLPPVISLANLGDKLCRLEF

SEQ ID NO: 135 - protein, 20 aa, Human herpesvirus 8
ISLANLGDKLCRLEFEYDSE

SEQ ID NO: 136 - protein, 20 aa, Human herpesvirus 8
LGDKLCRLEFEYDSEGDFVR

SEQ ID NO: 137 - protein, 20 aa, Human herpesvirus 8
CRLEFEYDSEGDFVRVPVAP

SEQ ID NO: 138 - protein, 20 aa, Human herpesvirus 8
EYDSEGDFVRVPVAPPEQPP

SEQ ID NO: 139 - protein, 20 aa, Human herpesvirus 8
GDFVRVPVAPPEQPPHVHLS

SEQ ID NO: 140 - protein, 20 aa, Human herpesvirus 8
VPVAPPEQPPHVHLSHFKKT

SEQ ID NO: 141 - protein, 20 aa, Human herpesvirus 8
PEQPPHVHLSHFKKTIQTIE

SEQ ID NO: 142 - protein, 20 aa, Human herpesvirus 8
HVHLSHFKKTIQTIEQATRE

SEQ ID NO: 143 - protein, 20 aa, Human herpesvirus 8
HFKKTIQTIEQATREATVAM

SEQ ID NO: 144 - protein, 20 aa, Human herpesvirus 8
IQTIEQATREATVAMTTIAK

SEQ ID NO: 145 - protein, 20 aa, Human herpesvirus 8
QATREATVAMTTIAKPIYPA

SEQ ID NO: 146 - protein, 20 aa, Human herpesvirus 8
ATVAMTTIAKPIYPAYIRLL

SEQ ID NO: 147 - protein, 20 aa, Human herpesvirus 8
TTIAKPIYPAYIRLLQRLEY

SEQ ID NO: 148 - protein, 20 aa, Human herpesvirus 8
PIYPAYIRLLQRLEYLNRLN

SEQ ID NO: 149 - protein, 20 aa, Human herpesvirus 8
YIRLLQRLEYLNRLNHHILR

SEQ ID NO: 150 - protein, 20 aa, Human herpesvirus 8
QRLEYLNRLNHHILRIPFPQ

SEQ ID NO: 151 - protein, 20 aa, Human herpesvirus 8
LNRLNHHILRIPFPQDALSE

SEQ ID NO: 152 - protein, 20 aa, Human herpesvirus 8
HHILRIPFPQDALSELQETY

SEQ ID NO: 153 - protein, 20 aa, Human herpesvirus 8
IPFPQDALSELQETYLAAFA

SEQ ID NO: 154 - protein, 20 aa, Human herpesvirus 8
DALSELQETYLAAFARLTKL

SEQ ID NO: 155 - protein, 20 aa, Human herpesvirus 8
LQETYLAAFARLTKLAADAA

SEQ ID NO: 156 - protein, 20 aa, Human herpesvirus 8
LAAFARLTKLAADAANTCSY

SEQ ID NO: 157 - protein, 20 aa, Human herpesvirus 8
RLTKLAADAANTCSYSLTKY

SEQ ID NO: 158 - protein, 20 aa, Human herpesvirus 8
AADAANTCSYSLTKYFGVLF

SEQ ID NO: 159 - protein, 20 aa, Human herpesvirus 8
NTCSYSLTKYFGVLFQHQLV

SEQ ID NO: 160 - protein, 20 aa, Human herpesvirus 8
SLTKYFGVLFQHQLVPTAIV

SEQ ID NO: 161 - protein, 20 aa, Human herpesvirus 8
FGVLFQHQLVPTAIVKKLLH

SEQ ID NO: 162 - protein, 20 aa, Human herpesvirus 8
QHQLVPTAIVKKLLHFDEAK

SEQ ID NO: 163 - protein, 20 aa, Human herpesvirus 8
PTAIVKKLLHFDEAKDTTEA

SEQ ID NO: 164 - protein, 20 aa, Human herpesvirus 8
KKLLHFDEAKDTTEAFLQSL

SEQ ID NO: 165 - protein, 20 aa, Human herpesvirus 8
FDEAKDTTEAFLQSLAQPVV

SEQ ID NO: 166 - protein, 20 aa, Human herpesvirus 8
DTTEAFLQSLAQPVVQGQRQ

SEQ ID NO: 167 - protein, 20 aa, Human herpesvirus 8
FLQSLAQPVVQGQRQGAAGG

SEQ ID NO: 168 - protein, 20 aa, Human herpesvirus 8
AQPVVQGQRQGAAGGSGVLT

SEQ ID NO: 169 - protein, 20 aa, Human herpesvirus 8
QGQRQGAAGGSGVLTQKELE

SEQ ID NO: 170 - protein, 20 aa, Human herpesvirus 8
GAAGGSGVLTQKELELLNKI

SEQ ID NO: 171 - protein, 20 aa, Human herpesvirus 8
SGVLTQKELELLNKINPQFT

SEQ ID NO: 172 - protein, 20 aa, Human herpesvirus 8
QKELELLNKINPQFTDAQAN

SEQ ID NO: 173 - protein, 20 aa, Human herpesvirus 8
LLNKINPQFTDAQANIPPSI

SEQ ID NO: 174 - protein, 20 aa, Human herpesvirus 8
NPQFTDAQANIPPSIKRSYS

SEQ ID NO: 175 - protein, 20 aa, Human herpesvirus 8
DAQANIPPSIKRSYSNKYDV

SEQ ID NO: 176 - protein, 20 aa, Human herpesvirus 8
IPPSIKRSYSNKYDVPEVSV

SEQ ID NO: 177 - protein, 20 aa, Human herpesvirus 8
KRSYSNKYDVPEVSVDWETY

SEQ ID NO: 178 - protein, 20 aa, Human herpesvirus 8
NKYDVPEVSVDWETYSRSAF

SEQ ID NO: 179 - protein, 20 aa, Human herpesvirus 8
PEVSVDWETYSRSAFEAPDD

SEQ ID NO: 180 - protein, 20 aa, Human herpesvirus 8
DWETYSRSAFEAPDDELRFV

SEQ ID NO: 181 - protein, 20 aa, Human herpesvirus 8
SRSAFEAPDDELRFVPLTLA

SEQ ID NO: 182 - protein, 20 aa, Human herpesvirus 8
EAPDDELRFVPLTLAGLRKL

SEQ ID NO: 183 - protein, 18 aa, Human herpesvirus 8
ELRFVPLTLAGLRKLFVE

SEQ ID NO: 184 - protein, 131 aa, Human herpesvirus 8
SRSNSTETAVTMPVSKERALAIYRVFAKGEVVAENTPILAFTDVELSTLKPHYLFIYDFIIEALCKSYTYSCTQARLESFLSRGIDFMTDLGQYLDTATSGKQQLTHSQIKEIKYRLLSCGLSASACDVFR

SEQ ID NO: 185 - protein, 137 aa, Homo sapiens
SLSHSAVKSLCKTLRRPRCLLETLRLAGCGLTAEDCKDLAFGLRANQTLTELDLSFNVLTDAGAKHLCQRLRQPSCKLQRLQLVSCGLTSDCCQDLASVLSASPSLKELDLQQNNLDDVGVRLLCEGLRHPACKLIR

SEQ ID NO: 186 - protein, 180 aa, Homo sapiens
PDYVEENRGHLIEIRDLFGPGLDTQEPRIVILQGAAGIGKSTLARQVKEAWGRGQLYGDRFQHVFYFSCRELAQSKVVSLAELIGKDGTATPAPIRQILSRPERLLFILDGVDEPGWVLQEPSSELCLHWSQPQPADALLGSLLGKTILPEASFLITARTTALQNLIPSLEQARWVEVLG

SEQ ID NO: 187 - protein, 122 aa, Human herpesvirus 8
PSEEVLAKTLVTEHHELFVSRSNSTETAVTMPVSKERALAIYRVFAKGEVVAENTPILAFTDVELSTLKPHYLFIYDFIIEALCKSYTYSCTQARLESFLSRGIDFMTDLGQYLDTATSGKQ

SEQ ID NO: 188 - protein, 928 aa, Human herpesvirus 8
MDGTDALEKLTKGLSGGGGSLHQTKLLMEFQLRGLPVPALLNSSTTEQFLNTVAQLPTDLSKFIRDYRVFALVRAAYFLEPPSSIDPLEAARALGRLVDILSSQPPQNTAPAQPPTSDDTLNNCTLLKLLAHYADQIAGFKTPALPPVPPGIIGLFTCVEQMYHACFQKYWAAALPPMWILTYDPPTSPLQDWLIVAYGNKEGLLLPSGIPSEEVLAKTLVTEHHELFVSRSNSTETAVTMPVSKERALAIYRVFAKGEVVAENTPILAFTDVELSTLKPHYLFIYDFIIEALCKSYTYSCTQARLESFLSRGIDFMTDLGQYLDTATSGKQQLTHSQIKEIKYRLLSCGLSASACDVFRTVIMTLPYRPTPNLANLSTFMGMVHQLTMFGHYFYRCLGSYSPTGLAFTELQKILTRASAEQTERNPWRHPGISDIPLRWKISRALAFFVPPAPINTLQRVYAALPSQLMRAIFEISVKTTWGGAVPANLARDIDTGPNTQHISSTPPPTLKDVETYCQGLRVGDTEYDEDIVRSPLFADAFTKSHLLPILREVLENRLQKNRALFQIRWLIIFAAEAATGLIPARRPLARAYFHIMDILEERHSQDALYNLLDCIQELFTHIRQAVPDAQCPHAFLQSLFVFQFRPFVLKHQQGVTLFLDGLQTSLPPVISLANLGDKLCRLEFEYDSEGDFVRVPVAPPEQPPHVHLSHFKKTIQTIEQATREATVAMTTIAKPIYPAYIRLLQRLEYLNRLNHHILRIPFPQDALSELQETYLAAFARLTKLAADAANTCSYSLTKYFGVLFQHQLVPTAIVKKLLHFDEAKDTTEAFLQSLAQPVVQGQRQGAAGGSGVLTQKELELLNKINPQFTDAQANIPPSIKRSYSNKYDVPEVSVDWETYSRSAFEAPDDELRFVPLTLAGLRKLFVE

SEQ ID NO: 189 - protein, 1473 aa, Homo sapiens
MAGGAWGRLACYLEFLKKEELKEFQLLLANKAHSRSSSGETPAQPEKTSGMEVASYLVAQYGEQRAWDLALHTWEQMGLRSLCAQAQEGAGHSPSFPYSPSEPHLGSPSQPTSTAVLMPWIHELPAGCTQGSERRVLRQLPDTSGRRWREISASLLYQALPSSPDHESPSQESPNAPTSTAVLGSWGSPPQPSLAPREQEAPGTQWPLDETSGIYYTEIREREREKSEKGRPPWAAVVGTPPQAHTSLQPHHHPWEPSVRESLCSTWPWKNEDFNQKFTQLLLLQRPHPRSQDPLVKRSWPDYVEENRGHLIEIRDLFGPGLDTQEPRIVILQGAAGIGKSTLARQVKEAWGRGQLYGDRFQHVFYFSCRELAQSKVVSLAELIGKDGTATPAPIRQILSRPERLLFILDGVDEPGWVLQEPSSELCLHWSQPQPADALLGSLLGKTILPEASFLITARTTALQNLIPSLEQARWVEVLGFSESSRKEYFYRYFTDERQAIRAFRLVKSNKELWALCLVPWVSWLACTCLMQQMKRKEKLTLTSKTTTTLCLHYLAQALQAQPLGPQLRDLCSLAAEGIWQKKTLFSPDDLRKHGLDGAIISTFLKMGILQEHPIPLSYSFIHLCFQEFFAAMSYVLEDEKGRGKHSNCIIDLEKTLEAYGIHGLFGASTTRFLLGLLSDEGEREMENIFHCRLSQGRNLMQWVPSLQLLLQPHSLESLHCLYETRNKTFLTQVMAHFEEMGMCVETDMELLVCTFCIKFSRHVKKLQLIEGRQHRSTWSPTMVVLFRWVPVTDAYWQILFSVLKVTRNLKELDLSGNSLSHSAVKSLCKTLRRPRCLLETLRLAGCGLTAEDCKDLAFGLRANQTLTELDLSFNVLTDAGAKHLCQRLRQPSCKLQRLQLVSCGLTSDCCQDLASVLSASPSLKELDLQQNNLDDVGVRLLCEGLRHPACKLIRLGLDQTTLSDEMRQELRALEQEKPQLLIFSRRKPSVMTPTEGLDTGEMSNSTSSLKRQRLGSERAASHVAQANLKLLDVSKIFPIAEIAEESSPEVVPVELLCVPSPASQGDLHTKPLGTDDDFWGPTGPVATEVVDKEKNLYRVHFPVAGSYRWPNTGLCFVMREAVTVEIEFCVWDQFLGEINPQHSWMVAGPLLDIKAEPGAVEAVHLPHFVALQGGHVDTSLFQMAHFKEEGMLLEKPARVELHHIVLENPSFSPLGVLLKMIHNALRFIPVTSVVLLYHRVHPEEVTFHLYLIPSDCSIRKAIDDLEMKFQFVRIHKPPPLTPLYMGCRYTVSGSGSGMLEILPKELELCYRSPGEDQLFSEFYVGHLGSGIRLQVKDKKDETLVWEALVKPGDLMPATTLIPPARIAVPSPLDAPQLLHFVDQYREQLIARVTSVEVVLDKLHGQVLSQEQYERVLAENTRPSQMRKLFSLSQSWDRKCKDGLYQALKETHPHLIMELWEKGSKKGLLPLSS

SEQ ID NO: 190 - protein, 74 aa, Rhesus monkey rhadinovirus
CKNQAYGCSRNAIEHFIKKGIDFMAELGAFIEKTCGYRSTVSLSNVRAVKARLASCGLSKEACEDFRAMILMTP

SEQ ID NO: 191 - protein, 69 aa, Homo sapiens
CKDLAFGLRANQTLTELDLSFNVLTDAGAKHLCQRLRQPSCKLQRLQLVSCGLTSDCCQDLASVLSASP

(19) United States
(12) Patent Application Publication
(10) Pub. No.: US 2013/0225479 A1
(43) Pub. Date: Aug. 29, 2013

(54) Methods and Compositions for Treating Inflammation

(71) Applicants: Blossom Damania, Chapel Hill, NC (US); Jenny P.-Y. Ting, Chapel Hill, NC (US); Sean Gregory, Hillsborough, NC (US)

(72) Inventors: Blossom Damania, Chapel Hill, NC (US); Jenny P.-Y. Ting, Chapel Hill, NC (US); Sean Gregory, Hillsborough, NC (US)

(73) Assignee: The University of North Carolina at Chapel Hill, Chapel Hill, NC (US)

(21) Appl. No.: 13/818,620

(22) PCT Filed: Aug. 26, 2011
(86) PCT No.: PCT/US2011/049309

§ 371 Date: Apr. 10, 2013

Related U.S. Application Data
(60) Provisional application No. 61/377,675, filed on Aug. 27, 2010.

(51) Int. Cl. A61K 38/10; A61K 45/06

(52) U.S. Cl. CPC A61K 38/10; A61K 45/06
USPC 514/1.4; 514/1.9; 514/15.1; 514/1.7; 514/1.1; 514/15.4; 530/326; 536/23.72; 435/235.1; 514/21.4; 514/13.2; 514/18.7; 514/16.8

(57) ABSTRACT

The present invention provides methods and compositions for treating inflammation and inflammatory disorders in a subject, by administering an effective amount of KSHV-Orf63 and/or active peptides and/or fragments thereof.

### PRIORITY STATEMENT

This application is a 35 U.S.C. §371 national phase entry of PCT Application PCT/US2011/049309, filed Aug. 26, 2011, which claims the benefit, under 35 U.S.C. §119(e) of U.S. Provisional Application Ser. No. 61/377,675, filed Aug. 27, 2010, the entire contents of each of-which are incorporated by reference herein.

### STATEMENT OF GOVERNMENT SUPPORT

Aspects of this invention were funded under Grant Nos. DE018281 and CA096500 awarded by the National Institutes of Health. The U.S. Government has certain rights in this invention.

### BACKGROUND OF THE INVENTION

Kaposi's sarcoma-associated herpesvirus (KSHV/HHV8) is the etiological agent of several human cancers including Kaposi's sarcoma (KS), primary effusion lymphoma (PEL) and multicentric Castleman's disease (MCD) (1, 2). The ability of KSHV to evade host innate immunity is essential for productive infection, latency and life-long persistence (3).

Several families of pattern recognition receptors (PRRs) have been described: Toll-like receptors (TLRs), nucleotide-binding and oligomerization, leucine-rich repeat (NLR) proteins, retinoic acid inducible gene (RIG)-1-like receptors (RLRs) and C-type lectin receptors (CLRs) (4, 5). Upon recognition of pathogen-associated molecular patterns (PAMPS), PRRs signal immune cell activation. TLRs play an important role in the lifecycle of KSHV (6, 7), but whether NLRs also do is unknown. Over twenty NLR family members have been identified in humans, and polymorphisms in several NLRs are linked to various autoinflammatory diseases (8-12). Activation of a subset of NLRs by PAMPs causes the formation of large multimeric complexes termed inflammasomes, which are composed of oligomers of a specific NLR, procaspase-1 and apoptotic-associated speck-like (ASC) adaptor protein (13). Inflammasome formation results in the proteolytic processing of proinflammatory cytokines IL-1β and IL-18 by active caspase-1. Excessive IL-1β and IL-18 production in response to pathogen infection is associated with pyroptosis, an inflammatory process involving caspase-1-mediated cell death.

The present invention provides methods and compositions comprising a KSHV Orf63 protein and/or active peptides and/or fragments thereof for the treatment of inflammation as well as inflammatory diseases and disorders.

### SUMMARY OF THE INVENTION

The present invention provides, in one aspect, a method of reducing inflammation in a subject, comprising administering to the subject an effective amount of a KSHV Orf63 protein or a biologically active fragment thereof and/or a KSHV Orf63 peptide, thereby reducing inflammation in the subject.

Further provided herein is a method of treating an inflammatory disorder in a subject in need thereof, comprising administering to the subject an effective amount of a KSHV Orf63 protein or a biologically active fragment thereof and/or a KSHV Orf63 peptide, thereby treating an inflammatory disorder in the subject.

In addition, the present invention provides a method of inhibiting activity in a subject of a proinflammatory molecule selected from the group consisting of interleukin-1β, interleukin-18, NLRP1, NLRP2 and any combination thereof, comprising administering to the subject an effective amount of a KSHV Orf63 protein or a biologically active fragment thereof and/or a KSHV Orf63 peptide, thereby inhibiting activity of the proinflammatory molecule in the subject.

The present invention additionally provides an isolated peptide comprising, consisting essentially of and/or consisting of the amino acid sequence as set forth in any of SEQ ID NOs:1-183 (Table 1) and any combination thereof (i.e., a peptide having an amino acid sequence of a KSHV Orf63 protein as set forth under the GenBank® Accession numbers provided herein and as are well known in the art). Further provided is a composition comprising the isolated peptide or combination thereof of this invention, with or without a full length KSHV Orf63 protein or biologically active fragment thereof, in a pharmaceutically acceptable carrier.

In further aspects, the present invention provides an isolated nucleic acid and/or a virus particle comprising a nucleotide sequence encoding a KSHV Orf63 protein or biologically active fragment thereof and/or an isolated peptide or combination thereof of this invention, which nucleic acid and/or virus particle can be present in a pharmaceutically acceptable carrier.

In additional aspects of this invention, a KSHV Orf63 peptide is provided in the compositions and methods of this invention, which can be a peptide comprising, consisting essentially of and/or consisting of an amino acid sequence of any of SEQ ID NOs:1-183 and in combination thereof and in particular embodiments, the KSHV Orf63 peptide of this invention can comprise, consist essentially of or consist of:

a) the amino acid sequence of SEQ ID NO:45;

b) the amino acid sequence of SEQ ID NO:46;

c) the amino acid sequence of SEQ ID NO:47;

d) the amino acid sequence of SEQ ID NO:48;

e) the amino acid sequence of SEQ ID NO:49;

f) the amino acid sequence of SEQ ID NO:50;

g) the amino acid sequence of SEQ ID NO:51;

h) the amino acid sequence of SEQ ID NO:52;

i) the amino acid sequence of SEQ ID NO:53;

j) the amino acid sequence of SEQ ID NO:54;

k) the amino acid sequence of SEQ ID NO:55;

l) the amino acid sequence of SEQ ID NO:56;

m) the amino acid sequence of SEQ ID NO:57;

n) the amino acid sequence of SEQ ID NO:58;

o) the amino acid sequence of SEQ ID NO:59;

p) the amino acid sequence of SEQ ID NO:60;

q) the amino acid sequence of SEQ ID NO:61;

r) the amino acid sequence of SEQ ID NO:62;

s) the amino acid sequence of SEQ ID NO:63;

t) the amino acid sequence of SEQ ID NO:64;

u) the amino acid sequence of SEQ ID NO:65;

v) the amino acid sequence of SEQ ID NO:66;

w) the amino acid sequence of SEQ ID NO:67;

x) the amino acid sequence of SEQ ID NO:68;

y) the amino acid sequence of SEQ ID NO:69;

z) the amino acid sequence of SEQ ID NO:70;

aa) the amino acid sequence of SEQ ID NO:71; and

bb) any combination of (a) through (aa) above.

### BRIEF DESCRIPTION OF THE DRAWINGS

FIGS. 1A-I. Orf63 is a viral homolog and inhibitor of NLRP1. (A-D) Stable Orf63 expression in THP-1 cells is detected by immunoblot (top panel in A). THP-1-Control and THP-1-Orf63 cells were either treated with vehicle control or primed with 5 ng/ml LPS for 1 hour, followed by stimulation with 10 μg/ml MDP for 6 hours. Supernatants were harvested and analyzed for IL-1β by enzyme linked immunosorbent assay (ELISA) and values were normalized to extracellular lactate dehydrogenase (LDH) released (A) or analyzed for pro-IL-1β and cleaved IL-1β by immunoblot (B). Supernatants were also analyzed for IL-18 by ELISA and normalized to extracellular LDH (C) and by immunoblot (D). (E) THP-1-Control or THP-1-Orf63 cells were treated as described above and extracellular LDH was calculated relative to positive control. (F) Procaspase-1, pro-IL-1β, ASC (together denoted as CIA) and NLRP1 expression plasmids were transfected into 293T cells with Orf63 or vector control. Cell extracts and supernatants were harvested 24 hours later and subjected to an IL-1β ELISA and immunoblot for cleaved IL-1β. (G) 293T cells were transfected with the indicated expression constructs followed by immunoblot for IL-1β expression. (H) NLRP1 and procaspase-1 were transfected into 293T cells with Orf63 or vector control for 24 hours. Caspase-1 enzymatic activity was determined by incubating lysates with caspase-1 substrate, WEHD-7-amino-4-trifluoromethyl coumarin (AFC). Fluorescence emission was measured every 15 minutes for 2 hours. (I) Caspase-1 was immunoprecipitated from 293T cells transfected with the indicated expression plasmids followed by immunoblot. ** indicates statistical significance P≦0.05 by two-tailed student's t-test. Data are representative of a minimum of three experimental replicates.

FIGS. 2A-E. Orf63 interacts with NLRP1. (A) 293T cells were transfected with expression plasmids for NLRP1, Orf63 or both plasmids and 48 hours later, NLRP1 was immunoprecipitated followed by immunoblotting for Orf63. (B) A reverse immunoprecipitation was performed. 293T cells were transfected with expression plasmids for NLRP 1, Orf63 or both plasmids and 48 hours later, Orf63 was immunoprecipitated followed by immunoblotting for NLRP1. (C) Co-immunoprecipitation of purified Orf63 and NLRP1-GST proteins followed by immunoblotting for Orf63-FLAG and GST-NLRP1. (D) 293T cells were transfected with Orf63 and full-length or mutant NLRP 1 expression plasmids followed by immunoprecipitation of Orf63 and immunoblotting for NLRP1. (E) 293T cells were transfected with Orf63-N and Orf63ΔN mutants and full-length NLRP1 expression plasmids for 48 hours. Immunoprecipitations were performed for NLRP1 followed by immunoblots for Orf63-N and Orf63ΔN mutants. Asterisk indicates non-specific band in input lanes. Data are representative of a minimum of three experimental replicates.

FIGS. 3A-H. Orf63 inhibits NLRP1 inflammasome formation and is necessary for IL-1β inhibition during viral infection. (A) Indicated expression plasmids were transfected into 293T cells. NLRP1 was immunoprecipitated 48 hours later and procaspase-1 interactions with NLRP 1 were determined by immunoblot. (B) 293T cells were transfected with indicated expression plasmids for 48 hours. NLRP1-myc was immunoprecipitated with anti-myc-antibody and interactions with NLRP1-V5 detected by immunoblot. (C) 293T cells were transfected with indicated expression plasmids followed by immunoprecipitation of either Orf63 or NOD2 48 hours later. Interaction of Orf63 with NOD2 was detected by immunoblot. (D) KSHV-infected primary human monocytes were transfected with siRNAs against Orf63 or non-targeting controls. 48 hours later, Orf63 and GAPDH transcription was analyzed by PCR. (E) Control and Orf63 siRNA monocytes were analyzed for IL-1β expression by ELISA 48 hours after transfection with Orf63 siRNA or non-targeting control siRNA. (F) Transcription of lytic viral genes, Orf49, Orf50 and Orf57, in KSHV-infected monocytes was analyzed by qPCR 48 hours after knock-down of Orf63. (G) BCBL-1 PEL cells were nucleofected with siRNAs against Orf63 or a non-targeting control. 24 hours later, the cells were treated with 25 ng/ml TPA. 96 hours post-TPA treatment, Orf49, Orf57 and Orf63 transcription was analyzed by qPCR. (H) BCBL-1 PEL cells were treated as in panel G, and 96 hours later, supernatants were used to infect naive Vero cells. 72 hours later, KSHV viral load in the infected Vero cells was determined by qPCR for Orf49. ** indicates statistical significance P≦0.05 by two-tailed student's t-test. Data are representative of a minimum of three experimental replicates.

FIGS. 4A-H. Orf63 inhibits the NLRP3 inflammasome. (A-D) THP-1-control or THP-1-Orf63 cells were mock treated or primed with 5 ng/ml LPS for 1 hour followed by stimulation with 2.5 mM ATP for 6 hours. Supernatants were harvested and analyzed by ELISA for IL-1β and normalized to extracellular LDH (A) or for pro-IL-1β and cleaved IL-1β by immunoblot (B). ATP-stimulated cells were also examined for IL-18 expression by ELISA and values were normalized to extracellular LDH (C) and pro-IL-18 and cleaved IL-18 by immunoblot (D). (E) THP-1-control or THP-1-Orf63 cells were treated with ATP as described above and subjected to an LDH release assay. (F) 293T cells were transfected with NLRP3 and Orf63 expression plasmids for 48 hours. NLRP3 was immunoprecipitated followed by immunoblotting for Orf63. (G) GST and GST-Orf63 was purified and subjected to SDS-PAGE and Coomassie staining. (H) GST or GST-Orf63 protein was incubated with glutathione beads and ³⁵S-methionine-labeled NLRP1, NLRP3 or luciferase protein as previously described (29). Complexes were washed several times and subjected to SDS-PAGE. The gel was dried and the bound radiolabeled proteins were detected by a phosphorimager. The first three lanes represent 20% of the input of each of the three labeled proteins added to the GST and GST-Orf63 binding reactions. ** indicates statistical significance P≦0.05 by two-tailed student's t-test. Data are representative of a minimum of three experimental replicates.

FIG. 5. Alignment of KSHV Orf63 with NLRP1. Amino acid alignments were performed using BLASTP and ClustalW2 bioinformatics programs with Orf63 (NCBI accession # YP_{—}001129420.1) and human NLRP1 (NCBI accession # NP_{—}127499). The LRR and NBD homology regions are shown in detail.

FIG. 6. Alignment of KSHV Orf63 with NLRP1. Amino acid sequence alignment of the full-length KSHV Orf63 and human NLRP 1 proteins performed using the ClustalW2 program is shown.

FIGS. 7A-D. KSHV Orf63 but not KSHV RTA inhibits NLRP 1-mediated IL-1β secretion. (A) 0.5×10⁶ THP-1 vector control cells or Orf63-expressing cells were primed with 5 ng/ml LPS for 1 hour followed by stimulation with 1 μg/ml, 10 μg/ml and 50 μg/ml MDP. Supernatants were harvested 6 hours later followed by an ELISA for extracellular IL-1β secretion. The IL-1β values were normalized to LDH. (B) THP1 cells were treated as described above with 10 μg/ml MDP and TNF-α expression was analyzed by ELISA. (C) NLRP1, procaspase-1, ASC and pro-IL-1β expression plasmids were transfected into 293T cells with Orf63, RTA or vector control. Supernatants were harvested 24h later and subjected to ELISA for extracellular IL-1β expression and expression of each component was depicted by immunoblot. (D) Immunoprecipitation of Orf63 from THP-1 cells followed by detection of endogenous NLRP1 by immunoblot using anti-NLRP1 antibody. Asterisk indicates non-specific band. **indicates statistical significance P≦0.05 by two-tailed student's t-test. Data are representative of a minimum of three experimental replicates.

FIGS. 8A-B. Orf63 does not interact with NLRP1 inflammasome components ASC and caspase-1 and blocks the interaction of caspase-1 with NLRP1. (A-B) 293T cells were transfected with expression plasmids for Orf63 and either ASC (A) or caspase-1 (B) and 48h later, immunoprecipitation of Orf63 (A) or caspase-1 (B) was performed by immunoblot for ASC or Orf63, respectively.

FIGS. 9A-D. The NBD of NLRP 1 is required, but not sufficient for interactions with Orf63. (A) Summary of NLRP1 mutants and Orf63's interactions with NLRP1. (B-D) Expression plasmids encoding PYD and CARD (B) or ΔPYD, PYD+NBD, ΔLRR, LRR and FIIND (C) or ΔCARD (D) mutants of NLRP1 were cotransfected into 293T cells with Orf63. Forty-eight hours post-transfection, Orf63 was immunoprecipitated and western blots for NLRP1 mutants were performed.

FIGS. 10A-B. Orf63-N and ORF63ΔN mutants are capable of inhibiting NLRP1 activity. (A) Panel depicting Orf63 mutants. Orf63-N, which contains amino acids 1-200, and Orf63ΔN, which contains amino acids 201-928. (B) NLRP 1 inflammasome was reconstituted by transfecting 293T cells with NLRP1, ASC, procaspase-1 and pro-IL-1β expression plasmids and wild-type Orf63, Orf63-N or Orf63ΔN mutants. Twenty four hours post-transfection, supernatants were analyzed for IL-1β by ELISA. ** indicates statistical significance P≦0.05 by two-tailed student's t-test. Data are representative of a minimum of three experimental replicates.

FIGS. 11A-C. Orf63 inhibits the interaction of procaspase-1 with NLRP1 and NLRP1 oligomerization. (A-B) Expression plasmids encoding NLRP1, ASC (A) and procaspse-1 (B) with either vector control or Orf63 were transfected into 293T cells. 48h later, NLRP1 was immunoprecipitated and immunoblots for each protein were performed. (C) 293T cells expressing NLRP1 or co-expressing NLRP1 and Orf63 were lysed under native conditions and subject to gel filtration analysis. Fractions were isolated and subjected to immunoblotting to detect the presence of NLRP1 and Orf63.

FIGS. 12A-B. NOD2 NBD is required for interactions with Orf63, and Orf63 does not interact with NOD1. (A) Full-length NOD2 or NOD2 mutants were transfected into 293T cells. 48h later, Orf63 was immunoprecipitated and NOD2 interactions detected by immunoblot. * indicates non-specific band. (B) 293T cells were transfected with Orf63 and NOD1 expression plasmids for 48 h. Orf63 was immunoprecipitated and the immunoprecipitates were subjected to western blot analysis for NOD1.

FIGS. 13A-B. Purity of primary human monocytes purified from healthy donors. (A-B) Human monocytes were isolated from peripheral blood mononuclear cells (PBMC) and analyzed by flow cytometry. Forward and side scatter (A) and CD14 expression (B) plots are shown to demonstrate purity. Percentage of cells that are CD14+ is determined by calculating the number of cells within the R2 marker.

FIGS. 14A-C. Orf63 inhibits IL-1β expression during KSHV reactivation. (A) KSHV-293T cells were transfected with RTA or vector control expression plasmids and either control siRNA (siControl) or Orf63-targeting siRNA (SiOrf63) as well as plasmids encoding procaspase-1, ASC and pro-IL-1β. RNA was harvested 24 hours later and cDNA was synthesized. Orf63 and GAPDH transcription levels were determined by PCR amplification and agarose gel electrophoresis. ˜indicates non-specific band. (B) KSHV-293T cells were transfected as described above and supernatants harvested 48 hours later. Extracellular IL-1β was determined by ELISA. (C) KSHV-293T cells were transfected with procaspe-1, pro-IL-1β, ASC, NLRP1 and either vector control or RTA expression plasmids. 72 hours post-transfection, reactivation and vIL-6 expression (indicator of lytic protein expression) was analyzed by immunoblot. ** indicates statistical significance P≦0.05 by two-tailed student's t-test. Data are representative of a minimum of three experimental replicates.

FIGS. 15A-C. NLRP1 inhibits KSHV reactivation from latency and production of infectious progeny virus. (A) BCBL-1-shRNA-control and BCBL-1-shRNA-NLRP1 stable knockdown cell lines were analyzed for NLRP1 expression by immunoblot. (B) BCBL-1 or BCBL-1-shRNA-NLRP1 stable knockdown PEL cells were mock treated or treated with 25 ng/ml TPA followed by analysis of viral genomes 96 hours post-treatment by qPCR. (C) BCBL-1-shRNA-control or BCBL-1-shRNA-NLRP1 PEL cells were treated as in panel B, and 96 hours later, supernatants were used to infect naïve Vero cells. 72 hours later, KSHV viral load in the infected Vero cell was determined by qPCR.

FIG. 16. Orf63 inhibits NLRP3 in dose-dependent manner. A dose-response graph for ATP treatment in control versus Orf63-expressing THP-1 cells. THP-1 controls cells or Orf63-expressing cells were stimulated with LPS as described followed by stimulation with 0.25 mM, 0.5 mM or 25 mM ATP. Supernatants were harvested 6 hours later and IL-1β secretion was analyzed by ELISA. The values were normalized to LDH. ** indicates statistical significance. P≦0.05 by two-tailed student's t-test. Data are representative of a minimum of three experimental replicates.

FIGS. 17A-E. Orf63 inhibits the NLRP3 inflammasome. (A-D) THP-1 control or THP-1-Orf63 cells were mock treated or primed with 5 ng/ml LPS for 1 hour followed by stimulation with 130 μg/ml Alum for 6 hours. Supernatants were harvested and analyzed by ELISA for IL-1β (A) or for pro-IL-1β and cleaved IL-1β by immunoblot (B). Alum-stimulated cells were also examined for IL-18 expression by ELISA (C) and pro-IL-18 and cleaved IL-18 by immunoblot (D). For both IL-1β and IL-18 ELISAs, values were normalized to LDH. (E) THP-1-control or THP-1-Orf63 cells were treated with ATP as described above and subjected to an LDH release assay. ** indicates statistical significance P≦0.05 by two-tailed student's t-test. Data are representative of a minimum of three experimental replicates.

FIG. 18. Rhesus monkey rhadinovirus (RRV) Orf63 demonstrates homology to NLRP1. Amino acid alignments were performed using BLASTP and ClustalW2 (depicted) bioinformatics programs with RRV Orf63 (NCBI accession# YP_{—}001129420.1) and human NLRP1 (NCBI accession# NP_{—}127499). The E value of the BlastP alignment of RRV Orf63 and NLRP1 was 0.002.

FIG. 19. Amino acid sequence of Orf63 (human herpesvirus 8; GenBank® Database Accession No. YP_{—}001129420).

### DETAILED DESCRIPTION OF THE INVENTION

Particular aspects of this invention are explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure that do not depart from the instant invention. Hence, the following specification is intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, nucleotide sequences, amino acid sequences and other references mentioned herein are incorporated by reference in their entirety.

The present invention is based on the unexpected discovery that KSHV Orf63, as well as active fragments and/or peptides of KSHV Orf 63, can be used to treat inflammation and inflammatory diseases and disorders.

### Embodiments of Compositions of this Invention

The present invention provides, in one aspect, an isolated peptide and/or fragment of a KSHV Orf63 protein that has activity in reducing inflammation and/or treating an inflammatory disease or disorder. Thus, in particular embodiments, the present invention provides an isolated peptide or fragment comprising, consisting essentially of and/or consisting of the amino acid sequence as set forth in any of SEQ ID NOs:1-183, and any combination thereof. For example, the present invention can comprise, consist essentially of, and/or consist of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182 or 183 of the peptides of Table 1 in any combination and/or ratio relative to one another and in any association with one another (e.g., as single peptides, as linked peptides and/or as a combination of both single and linked peptides, which can include any number and combination of peptides including repeats, linked in any order).

The peptides set forth in SEQ ID NOs:1-183 are based on a 928 amino acid KSHV Orf63 protein (e.g., as provided as GenBank® Accession No. YP_{—}001129420).

The term “equivalent” in some embodiments of this invention means a KSHV Orf63 peptide made up of or comprising amino acids that correspond to the same or similarly numbered amino acids in a peptide from a different organism or that the KSHV Orf63 peptide has substantially similar identity or homology to a peptide of a different organism (e.g., 60%, 70%, 75%, 80%, 85%, 90%, 95%, 97%, 98%, 99%, 100%). The term “equivalent” is also intended in some embodiments to mean a peptide of a KSHV Orf63 protein having the same or similar biological activity or function as a peptide of a different organism. Such equivalent peptides would be readily produced and analyzed by one of ordinary skill in the art according to standard and well known methods, as well as according to the methods described herein.

Thus, in some embodiments, the present invention provides a peptide comprising, consisting essentially of and/or consisting of amino acids 1-20, 5-25, 10-30, 15-35, 20-40, 25-45, 30-50, 35-55, 40-60, 45-65, 50-70, 55-75, 60-80, 65-85, 70-90, 75-95, 80-100, 85-105, 90-110, 95-115, 100-120, 105-125, 110-130, 115-135, 120-140, 125-145, 130-150, 135-155, 140-160, 145-165, 150-170, 155-175, 160-180, 165-185, 170-190, 175-195, 180-200, 185-205, 190-210, 195-215, 200-220, 205-225, 210-230, 215-235, 220-240, 225-245, 230-250, 235-355, 240-260, 245-265, 250-270, 255-275, 260-280, 265-285, 270-290, 275-295, 280-300, 285-305, 290-310, 295-315, 300-320, 305-325, 310-330, 315-335, 320-340, 325-345, 330-350, 335-355, 340-360, 345-365, 350-370, 355-375, 360-380, 365-385, 370-390, 375-395, 380-400, 385-405, 390-410, 395-415, 400-420, 405-425, 410-430, 415-435, 420-440, 425-445, 430-450, 435-455, 440-460, 445-465, 450-470, 455-475, 460-480, 465-485, 470-490, 475-495, 480-500, 485-505, 490-510, 495-515, 500-520, 505-525, 510-530, 515-535, 520-540, 525-545, 530-550, 535-555, 540-560, 545-565, 550-570, 555-575, 560-580, 565-585, 570-590, 575-595, 580-600, 585-605, 590-610, 595-615, 600-620, 605-625, 610-630 615-635, 620-640, 625-645, 630-650, 635-655, 640-660, 645-665, 650-670, 655-675, 660-680, 665-685, 670-690, 675-695, 680-700, 685-705, 690-710, 695-715, 700-720, 705-725, 710-730, 715-735, 720-740, 725-745, 730-750, 735-755, 740-760, 745-765, 750-770, 755-775, 760-780, 765-785, 770-790, 775-795, 780-800, 785-805, 790-810, 795-815, 800-820, 805-825, 810-830, 815-835, 825-845, 835-855, 845-865, 855-875, 865-885, 875-895, 895-915 and/or 905-925, singly or in any combination, of a KSHV Orf63 protein of this invention (e.g., as set forth according to the numbering of amino acids in the amino acid sequence identified by the GenBank® Accession number YP_{—}001129420, as set forth herein, which is incorporated by reference herein in its entirety, and as otherwise known in the art).

The present invention further provides a domain or fragment (e.g., a biologically active domain or fragment) of a KSHV Orf63 protein as described herein. Such a domain or fragment of this invention can comprise, consist essentially of and/or consist of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910 or 920 contiguous amino acids of the KSHV Orf63 protein of this invention (e.g., as set forth pursuant to the numbering of the amino acid sequence identified by the GenBank® Accession number YP_{—}001129420, as set forth herein and as otherwise known in the art), starting from either the amino terminus, the carboxy terminus and/or any internal site and in any combination. Furthermore, the domain or fragment of this invention can be combined with any other domain or fragment, either in operable association therewith, as separate domains or fragments (e.g., in a composition) or both. As one nonlimiting example, a 30 amino acid fragment near the amino terminus of the KSHV Orf63 protein can be combined, either in operable association with or as part of a composition with, a different 20 amino acid fragment that may also be near the amino terminus or it may be near the carboxy terminus.

Further provided is a composition comprising, consisting essentially of and/or consisting of an isolated peptide or combination thereof of this invention, with or without a full length KSHV Orf63 protein and/or biologically active fragment thereof (e.g., a fragment of the KSHV Orf63 protein that has at least one activity of the full length KSHV Orf63 protein), in a pharmaceutically acceptable carrier. Also provided herein is a composition comprising, consisting essentially of and/or consisting of a fragment or domain of a KSHV Orf63 protein, with or without a full length KSHV Orf63 protein. Such compositions can further comprise any of the delivery components and/or biological agents of this invention, as described herein. In particular, the compositions of this invention can comprise other therapeutic agents (e.g., anti-inflammatory agents) that reduce inflammation and/or treat inflammatory disease, as would be known to one of ordinary skill in the art.

In further aspects, the present invention provides an isolated nucleic acid and/or virus particle comprising a nucleotide sequence encoding a KSHV Orf63 protein and/or biologically active fragment thereof and/or an isolated peptide or combination thereof of this invention, any of which can be present in a pharmaceutically acceptable carrier.

### Embodiments of Methods of the Invention

The present invention is based on the discovery that KSHV Orf63 and/or active peptides and/or active fragments of KSHV Orf63, as well as nucleic acids encoding any of these, can be administered to a subject to treat or reduce inflammation and/or treat or prevent an inflammatory disease or disorder.

Thus in one aspect, the present invention provides a method of treating and/or reducing inflammation in a subject, comprising administering to the subject an effective amount of a KSHV Orf63 protein or a biologically active fragment thereof and/or a KSHV Orf63 peptide, thereby reducing inflammation in the subject.

Further provided herein is a method of treating and/or preventing an inflammatory disease or disorder in a subject in need thereof, comprising administering to the subject an effective amount of a KSHV Orf63 protein or a biologically active fragment thereof and/or a KSHV Orf63 peptide, thereby treating an inflammatory disease or disorder in the subject.

In addition, the present invention provides a method of inhibiting activity in a subject of a proinflammatory molecule, which can be but is not limited to, interleukin-10, interleukin-18, NLRP1, NLRP3, any other NLR protein family member and any combination thereof, comprising administering to the subject an effective amount of a KSHV Orf63 protein or a biologically active fragment thereof and/or a KSHV Orf63 peptide, thereby inhibiting activity of the proinflammatory molecule in the subject.

In the methods of this invention, in some embodiments, the KSHV Orf63 protein or biologically active fragment thereof and/or peptide can be administered directly to a site of inflammation in the subject. In some embodiments, the KSHV Orf63 protein or biologically active fragment thereof and/or peptide can be administered to the subject intravenously, intra-arterially, orally and/or transdermally.

Some aspects of this invention include administering an anti-inflammatory agent to the subject in addition to the KSHV Orf63 protein or biologically active fragment thereof and/or peptide. Such agents can be administered before, concurrently with and/or after administration of the KSHV Orf63 protein or biologically active fragment thereof and/or peptide.

Non-limiting examples of an inflammatory disorder that can be treated by the methods of this invention include atherosclerosis, ulcerative colitis, inflammatory bowel disease, Crohn's disease, pancreatitis, pelvic inflammatory disease, rheumatoid arthritis, osteoarthritis, asthma, hay fever, seasonal allergies, perennial allergies, vasculitis, psoriasis, blisters, allergic rhinitis, peptic ulcer disease, acne vulgaris, dermatitis, hypersensitivities, glomerulonephritis, sarcoidosis, inflammation-associated cancer, transplant rejection, gout, postoperative intra-abdominal sepsis, ischemia-reperfusion injury, pancreatic and liver damage, sepsis and septic shock, gastric damage caused by certain drugs, stress-induced gastric damage, gastric damage caused by H. pylori, inflammatory pain, chronic kidney disease, intestinal inflammation, autoimmune disease (including but not limited to familial cold autoinflammatory syndrome, Muckle Wells syndrome and neonatal onset multisystem inflammatory disease (NOMID), vitiligo and any combination thereof.

In some embodiments of this invention, the inflammation to be reduced and/or treated can be caused by, e.g., infection, trauma, autoimmune dysfunction, autoinflammatory disorder, autoimmune disorder, genetic predisposition, allergic reaction, surgical wounding, burn, tissue damage, tissue incision and any combination thereof.

As noted above, in some embodiments of the methods of this invention, the KSHV Orf63 protein or biologically active fragment thereof and/or peptide and/or nucleic acid and/or virus particle and/or composition can be administered directly to an injury/trauma/wound/surgical site in the subject.

In the methods of this invention, an effective amount of the KSHV Orf63 protein or biologically active fragment thereof or the peptide is in the range of about 1 microgram/ml to about 500 milligrams/ml, with the optimum dosage for a given subject being routinely determined according to methods standard in the art (see, e.g., Remington's Pharmaceutical Sciences, latest edition).

The methods of this invention can further comprise delivering an effective amount of an anti-inflammatory agent, and effective amount of an antimicrobial agent, an effective amount of a tissue regeneration agent, an effective amount of a cytokine, or any combination thereof to the subject or to the injury/trauma/surgical/wound site of the subject to reduce and/or prevent inflammation and damage to tissue surrounding the site. Nonlimiting examples of an anti-inflammatory agent of this invention include steroids and nonsteroid anti-inflammatory agents as are well known in the art. Nonlimiting examples of a cytokine of this invention include anti-inflammatory cytokines such as IL-10, IL-4, IL-11, IL1Ra, TGF-β, osteoprotegerin and any combination thereof.

Further non-limiting examples of anti-inflammatory agent of this invention include an inhibitor of interleukin-1 (IL-1), an anti-IL-1 antibody, an inhibitor of interleukin-6 (IL-6), an inhibitor of tumor necrosis factor alpha (TNF-α), an inhibitor of Caspase-1, an inhibitor of NF-KB or members of the NF-KB pathway that activate NF-KB, an inhibitor of matrix metalloproteinase (MMP) 1, 2, 8 and/or 9, an inhibitor of p38 mitogen activated protein kinase (MAPK), an inhibitor of extracellular signal-related kinase (ERK) (ERK1; ERK2), SBR203580 (p38 inhibitor), PD98059 (ERK inhibitor), U0126 (inhibitor of MMP expression) simvastatin (inhibitor of MMP-1 expression), and any combination thereof.

The anti-inflammatory agents, antimicrobial agents, tissue regeneration agents and cytokines of this invention can be delivered to the subject as a protein or active fragment thereof and/or as a nucleic acid encoding the protein or active fragment or peptide thereof. The amino acid sequences and nucleic acid sequences of exemplary anti-inflammatory agents and cytokines of this invention, as well as active fragments thereof are well known in the art and would be readily available to those skilled in the art. The KSHV Orf63 protein, peptides and/or fragments and/or nucleic acids of this invention, as well as the anti-inflammatory agents and cytokines, either as proteins or nucleic acids, can be administered in any combination and in any order relative to one another and in any time frame relative to one another.

In further embodiments of this invention, it is contemplated that a nucleic acid of this invention can be delivered to a subject of this invention, wherein the nucleic acid encodes a KSHV Orf63 protein, a peptide and/or fragment of this invention, and an antagonist of a pro-inflammatory agent. In some embodiments, the nucleic acid can be under the control of a promoter and/or other regulatory element such that expression of the nucleic acid is induced by a pro-inflammatory agent to be expressed to produce the KSHV Orf63 protein, peptide and/or fragment and antagonist of the pro-inflammatory agent. Nonlimiting examples of antagonists of pro-inflammatory agents include antagonists of TNFα, CSF-1, IL-6, IL 12, IL 17, IL1B, receptor activator of nuclear factor-kappa B (RANK), RANK ligand (RANKL) and combinations thereof.

As used herein, the term “antimicrobial agent” means any agent that kills, inhibits the growth of, or prevents the growth of a bacterium (including mycoplasma), fungus, yeast, or virus. Suitable antimicrobial agents of this invention include, but are not limited to, antibiotics such as vancomycin, bleomycin, pentostatin, mitoxantrone, mitomycin, dactinomycin, plicamycin and amikacin. Other antimicrobial agents include, but are not limited to, antibacterial agents such as 2-p-sulfanilyanilinoethanol, 4,4′-sulfinyldianiline, 4-sulfanilamidosalicylic acid, acediasulfone, acetosulfone, amikacin, amoxicillin, amphotericin B, ampicillin, apalcillin, apicycline, apramycin, arbekacin, aspoxicillin, azidamfenicol, azithromycin, aztreonam, bacitracin, bambermycin(s), biapenem, brodimoprim, butirosin, capreomycin, carbenicillin, carbomycin, carumonam, cefadroxil, cefamandole, cefatrizine, cefbuperazone, cefclidin, cefdinir, cefditoren, cefepime, cefetamet, cefixime, cefinenoxime, cefininox, cefodizime, cefonicid, cefoperazone, ceforanide, cefotaxime, cefotetan, cefotiam, cefozopran, cefpimizole, cefpiramide, cefpirome, cefprozil, cefroxadine, ceftazidime, cefteram, ceftibuten, ceftriaxone, cefuzonam, cephalexin, cephaloglycin, cephalosporin C, cephradine, chloramphenicol, chlortetracycline, ciprofloxacin, clarithromycin, clinafloxacin, clindamycin, clindamycin phosphate, clomocycline, colistin, cyclacillin, dapsone, demeclocycline, diathymosulfone, dibekacin, dihydrostreptomycin, dirithromycin, doxycycline, enoxacin, enviomycin, epicillin, erythromycin, flomoxef, fortimicin(s), gentamicin(s), glucosulfone solasulfone, gramicidin S, gramicidin(s), grepafloxacin, guamecycline, hetacillin, imipenem, isepamicin, josamycin, kanamycin(s), leucomycin(s), lincomycin, lomefloxacin, lucensomycin, lymecycline, meclocycline, meropenem, methacycline, micronomicin, midecamycin(s), minocycline, moxalactam, mupirocin, nadifloxacin, natamycin, neomycin, netilmicin, norfloxacin, oleandomycin, oxytetracycline, p-sulfanilylbenzylamine, panipenem, paromomycin, pazufloxacin, penicillin N, pipacycline, pipemidic acid, polymyxin, primycin, quinacillin, ribostamycin, rifamide, rifampin, rifamycin SV, rifapentine, rifaximin, ristocetin, ritipenem, rokitamycin, rolitetracycline, rosaramycin, roxithromycin, salazosulfadimidine, sancycline, sisomicin, sparfloxacin, spectinomycin, spiramycin, streptomycin, succisulfone, sulfachrysoidine, sulfaloxic acid, sulfamidochrysoidine, sulfanilic acid, sulfoxone, teicoplanin, temafloxacin, temocillin, tetracycline, tetroxoprim, thiamphenicol, thiazolsulfone, thiostrepton, ticarcillin, tigemonam, tobramycin, tosufloxacin, trimethoprim, trospectomycin, trovafloxacin, tuberactinomycin and vancomycin. Exemplary antimicrobial agents may also include, but are not limited to, anti-fungals, such as amphotericin B, azaserine, candicidin(s), chlorphenesin, dermostatin(s), filipin, fungichromin, mepartricin, nystatin, oligomycin(s), perimycin A, tubercidin, imidazoles, triazoles, and griesofulvin. Further exemplary antimicrobial agents can include, but are not limited to anti-virals, such as acyclovir, valacyclovir, famcyclovir, gancyclovir, amantadine and others known in the art.

An antimicrobial agent of this invention can also be an antimicrobial peptide, including a plant derived antimicrobial peptide and/or an animal antimicrobial peptide. Antimicrobial peptides (AMPs) are short sequence peptides with generally fewer than 50 amino acid residues, which have antimicrobial activity against microorganisms. They are a first line of defense in plants and animals which are ubiquitous in nature with high selectivity against target organisms, and resistance against them is much less observed compared with current antibiotics (Zasloff, 2002).

AMPs are diverse and can be subdivided into two major groups based on their electrostatic charges, which are the most important characteristic of AMPs (Vizioli and Salzet, 2002). The largest group of AMPs is that of cationic molecules, which are wildly distributed in plants and animals. The much smaller group of AMPs is that of non-cationic molecules including anionic peptides, aromatic peptides and peptides derived from oxygen-binding proteins. Compared with the first group, the non-cationic peptides are scarce and often the term “antimicrobial peptides (AMPs)” is used to refer only to cationic AMPs (Zasloff, 2002; Keymanesh, 2009).

On the basis of structural features, cationic AMPs can be subdivided into three classes: (1) linear peptides often adopting α-helical structures; (2) cysteine-rich open-ended peptides containing a single or several disulfide bridges; and (3) cyclopeptides forming a peptide ring (Montesinos, 2007). However, they also share certain common structural characteristics such as (1) amino acid composition in which cationic and hydrophobic residues are most abundant; (2) amphipathicity; and (3) a remarkable diversity of structures and conformations even including some non-conventional and extended structures (Vizioli and Salzet, 2002; Keymanesh, 2009). In fact, the second characteristic, amphipathicity, in many cases is membrane-induced, and this is an important property of cationic AMPs which can facilitate their interactions with microbial membranes (Zasloff, 2002). Some cationic AMPs are enriched in certain amino acids. For example, many cationic AMPs are rich in cysteines forming a single or several disulfide bridges (e.g., Ib-AMP4 from balsamine and penaeidins from shrimp), which makes their structures more compact and stable under various biochemical conditions such as protease degradation and so on. This group of AMPs is widespread in nature, including plants, animals, insects, and fungi, and exhibit a significant sequence and structure diversity (Vizioli and Salzet, 2002).

As used herein, the term “tissue regeneration agent” describes an agent or molecule that functions to promote, initiate and/or enhance regeneration of tissue, including but not limited to, skin, bone, tooth, muscle, connective tissue, cells, cartilage, tendon, ligament, mucous membrane and any combination thereof. A regenerative agent or biomolecule of the present invention includes, but is not limited to, a differentiation stimulating biomolecule, a chemotaxis stimulating molecule, a proliferation stimulating biomolecule, a mobilization stimulating biomolecule, or any combination thereof.

In some embodiments of the invention, the differentiation stimulating biomolecule includes, but is not limited to, a bone morphogenic protein (BMP, including BMP-1, BMP-2, BMP-4, BMP-5, BMP-6, BMP-7, BMP-8a and/or BMP-9), a transforming growth factor (TGF), including TGF-alpha, TGF-beta 1, TGF-beta 2 and TGF-beta 3, vitamin B12, an insulin-like growth factor-I (e.g., IGF-I; Stem Cells 22:1152-1167 (2004)), IGF-II, or any combination thereof.

In other embodiments, the chemotaxis and/or proliferation stimulating biomolecule includes, but is not limited to, a hepatocyte growth factor (HGF), a stromal cell-derived growth factor-1 (SDF-1), a platelet derived growth factor-bb (PDGF-bb), an insulin-like growth factor (IGF), including IGF-I and IGF-II, an insulin-like growth factor binding protein (IGFBP), including IGFBP-1, IGFBP-2, IGFBP-3, IGFBP-4, IGFBP-5, IGFBP-6, IGFBP-7, TGF-beta 1, TGF-beta 3, BMP 2, BMP 4, BMP 7, basic fibroblast growth factor (bFGF), an interleukin (e.g., interleukin-8; interleukin-10) or any combination thereof.

In further embodiments of the invention, the mobilization stimulating biomolecule includes, but is not limited to, a hepatocyte growth factor (HGF), a stromal cell-derived growth factor-1 (SDF-1), a platelet derived growth factor-bb (PDGF-bb), an insulin-like growth factor (IGF), including IGF-I and IGF-II, an insulin-like growth factor binding protein (IGFBP), including IGFBP-1, IGFBP-2, IGFBP-3, IGFBP-4, IGFBP-5, IGFBP-6, IGFBP-7, TGF-beta 1, TGF-beta 3, BMP 2, BMP 4, BMP 7, basic fibroblast growth factor (bFGF), FGF, EGF, an interleukin (e.g., interleukin-8; interleukin-10) or any combination thereof.

In still further embodiments, the bone morphogenic protein (BMP) includes, but is not limited to, BMP-2, BMP-4, BMP-5, BMP-6, BMP-7, or any combination thereof. In yet other aspects of the invention, the transforming growth factor (TGF) includes, but is not limited to, TGF β-1, TGF β-3, or any combination thereof. In other aspects of the invention, the insulin-like growth factor (IGF) includes, but is not limited to, IGF-I, IGF-II, or any combination thereof. Thus, in particular aspects of the invention, the differentiation stimulating biomolecule that is an insulin-like growth factor is IGF-I. In other aspects of the invention, the chemotaxis and/or proliferation stimulating biomolecule that is an insulin-like growth factor is IGF-I, IGF-II, or any combination thereof. In further embodiments, the insulin-like growth factor binding protein (IGFBP) includes but is not limited to IGFBP-3, IGFBP-5, or any combination thereof. In still further embodiments, the interleukin is selected from the group consisting of IL-8, IL-10, or any combination thereof.

The present invention also provides various compositions. In some embodiments these compositions can be employed, e.g., in the methods described herein. Thus, the present invention provides a composition comprising, consisting essentially of and/or consisting of a KSHV Orf63 protein, a peptide and/or active fragment thereof and/or a nucleic acid encoding a KSHV Orf63 protein, peptide and/or active fragment thereof, which can be, for example, in a pharmaceutically acceptable carrier. Such compositions of this invention can further comprise, consist essentially of and/or consist of an anti-inflammatory agent, a cytokine, an immune modulator, an antagonist of a pro-inflammatory agent or any combination thereof and/or a nucleic acid encoding an anti-inflammatory agent, a cytokine, an immune modulator, an antagonist of a pro-inflammatory agent, a differentiation-stimulating agent, a chemotaxis stimulating agent, a proliferation stimulating agent, a mobilization stimulating agent or any combination thereof.

It is further contemplated that the present invention provides a kit comprising, consisting essentially of and/or consisting of compositions of this invention. It would be well understood by one of ordinary skill in the art that the kit of this invention can comprise one or more containers and/or receptacles to hold the reagents (e.g., KSHV Orf63 proteins, peptides and/or active fragments thereof, nucleic acids, viral vectors, etc.) of the kit, along with appropriate buffers and/or diluents and/or other solutions and directions for using the kit, as would be well known in the art. Such kits can further comprise anti-inflammatory agents, antagonists of pro-inflammatory agents and/or other cytokines, as well as nucleic acids encoding the same, in any combination, as described herein and as are well known in the art.

The compositions and kits of the present invention can also include other medicinal agents, pharmaceutical agents, carriers and diluents, etc. Actual methods of preparing such dosage forms are known, or will be apparent, to those skilled in this art.

In the kits of this invention, the compositions can be presented in unit\dose or multi-dose containers, for example, in sealed ampules and vials, and can be stored in a freeze-dried (lyophilized) condition requiring only the addition of the sterile liquid carrier, for example, saline or water-for-injection immediately prior to use.

### FURTHER DEFINITIONS

The following terms are used in the description herein and the appended claims:

As used herein, “a,” “an,” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

The present invention, as well as the term “KSHV Orf63,” encompasses any peptide, polypeptide, protein, analog, isoform or derivative of KSHV Orf63, the nucleic acid sequence and amino acid sequence of which is well known in the art.

Exemplary peptides of this invention are listed in Table 1. The KSHV Orf63 peptide, polypeptide, protein, isoform, analog and/or derivative thereof used in the present invention may be present in any amount that is sufficient to elicit a beneficial and/or therapeutic effect and, where applicable, may be present either substantially in the form of one optically pure enantiomer or as a mixture, racemic or otherwise, of enantiomers. As will be appreciated by those skilled in the art, the actual amount of peptide, polypeptide, protein, analogs and/or derivatives thereof used in the compositions of this invention will depend on the potency of the selected compound in question. The peptides, polypeptides, proteins, analogs and/or derivatives described herein may be obtained through commercial resources or may be prepared according to methods known to one skill in the art.

As used herein, “nucleic acid,” “nucleotide sequence” and “polynucleotide” encompass both RNA and DNA, including cDNA, genomic DNA, mRNA, synthetic (e.g., chemically synthesized) DNA and chimeras of RNA and DNA [e.g., DNA-RNA hybrid sequences (including both naturally occurring and non-naturally occurring nucleotides)], but are typically either single or double stranded DNA or RNA sequences.

The term polynucleotide or nucleotide sequence refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid or nucleotide sequence of this invention.

An “isolated nucleic acid” is a nucleotide sequence (e.g., DNA or RNA) that is not immediately contiguous with nucleotide sequences with which it is immediately contiguous (one on the 5′ end and one on the 3′ end) in the naturally occurring genome or environment of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by oligonucleotide synthesis, PCR or restriction endonuclease treatment), independent of other sequences. It also includes a recombinant DNA that is part of a hybrid nucleic acid encoding an additional polypeptide or peptide sequence.

The term “isolated” can refer to a nucleic acid, nucleotide sequence, polypeptide, peptide or fragment that is at least partially and in some embodiments substantially free of cellular material, viral material, and/or culture medium (e.g., when produced by recombinant DNA techniques), or chemical precursors or other chemicals (e.g., when chemically synthesized). Moreover, an “isolated fragment” is a fragment of a nucleic acid, nucleotide sequence or polypeptide that is not naturally occurring as a fragment and would not be found as such in the natural state. “Isolated” does not mean that the preparation is technically pure (homogeneous), but it is sufficiently pure to provide the polypeptide, peptide or nucleic acid in a form in which it can be used for the intended purpose.

An “isolated cell” refers to a cell that is at least partially separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated ex vivo and then returned to the subject.

The term “nucleic acid fragment” will be understood to mean a nucleotide sequence of reduced length relative to a reference nucleic acid or nucleotide sequence and comprising, consisting essentially of and/or consisting of a nucleotide sequence of contiguous nucleotides identical or almost identical (e.g., 60%, 65%, 70%, 75%, 80%, 85%, 90%, 92%, 95%, 98%, 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially of and/or consist of, oligonucleotides having a length of at least about 6, 7, 8, 9, 10, 11, 12, 13, 14, 15, 16, 17, 18, 19, 20, 21, 22, 23, 24, 25. 50, 75, 100, 150, 200, 250, 300, 350, 400, 450, 500, 750, 1000, 1500, 2000, 2500, 3000, 4000 or 5000 consecutive nucleotides of a nucleic acid or nucleotide sequence according to the invention.

Several methods known in the art may be used to produce a polynucleotide and/or vector according to this invention. A “vector” is any nucleic acid molecule for the cloning and/or amplification of nucleic acid as well as for the transfer of nucleic acid into a subject (e.g., into a cell of the subject). A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in vivo, i.e., capable of replication under its own control. The term “vector” includes both viral and nonviral nucleic acid molecules for introducing a nucleic acid into a cell in vitro, ex vivo, and/or in vivo.

A large number of vectors known in the art may be used to manipulate nucleic acids, incorporate response elements and promoters into genes, nucleotide sequences, coding sequences, etc. Such vectors include, for example, plasmids or modified viruses including, for example bacteriophages such as lambda derivatives, or plasmids such as pBR322 or pUC plasmid derivatives, or the Bluescript® vector. For example, the insertion of the nucleic acid fragments or segments that function as response elements and promoters into a suitable vector can be accomplished by ligating the appropriate nucleic acid fragments into a chosen vector that has complementary cohesive termini. Alternatively, the ends of the nucleic acid molecules may be enzymatically modified or any site may be produced by ligating nucleotide sequences (linkers) to the nucleic acid termini. Such vectors may be engineered to contain sequences encoding selectable markers that provide for the selection of cells that contain the vector and/or have incorporated the nucleic acid of the vector into the cellular genome. Such markers allow identification and/or selection of host cells that incorporate the nucleic acid and produce the proteins encoded by the marker,

Vectors have been used in a wide variety of gene delivery applications in cells, as well as in living animal subjects. Viral vectors that can be used include but are not limited to retrovirus, lentivirus, adeno-associated virus, poxvirus, alphavirus, baculovirus, vaccinia virus, herpes virus, Epstein-Barr virus, and adenovirus vectors, as well as any combination thereof. Nonlimiting examples of non-viral vectors include plasmids, liposomes, electrically charged lipids (cytofectins), nucleic acid-protein complexes, and biopolymers, as well as any combination thereof. In addition to a nucleic acid of interest, a vector may also comprise one or more regulatory regions (e.g., promoters, enhancers, termination sequences, etc.), and/or selectable markers useful in selecting, measuring, and monitoring nucleic acid transfer results (delivery to specific tissues, duration of expression, etc.).

“Promoter” refers to a nucleic acid sequence capable of controlling the expression of a coding sequence or functional RNA. In general, a coding sequence is located 3′ to a promoter sequence. Promoters may be derived in their entirety from a native sequence, or be composed of different elements derived from different promoters found in nature, or even comprise synthetic nucleic acid segments. It is understood by those skilled in the art that different promoters may direct the expression of a nucleotide sequence in different tissues or cell types and/or at different stages of development and/or in response to different environmental or physiological conditions.

Promoters that cause a nucleotide sequence to be expressed in most cell types at most times are commonly referred to as “constitutive promoters.” Promoters that cause a nucleotide sequence to be expressed in a specific cell type are commonly referred to as “cell-specific promoters” or “tissue-specific promoters.” Promoters that cause a nucleotide sequence to be expressed at a specific stage of development or cell differentiation are commonly referred to as “developmentally-specific promoters” or “cell differentiation-specific promoters.” Promoters that are induced and cause a nucleotide sequence to be expressed following exposure or treatment of the cell with an agent, biological molecule, chemical, ligand, light, or the like that induces the promoter are commonly referred to as “inducible promoters” or “regulatable promoters.” It is further recognized that, because in most cases the exact boundaries of regulatory sequences have not been completely defined, nucleotide sequences of different lengths may have identical promoter activity.

A “promoter sequence” is a nucleic acid regulatory region capable of binding RNA polymerase in a cell and initiating transcription of a downstream (3′ direction) coding sequence. For purposes of defining the present invention, the promoter sequence is bound at its 3′ terminus by the transcription initiation site and extends upstream (5′ direction) to include the minimum number of bases or elements necessary to initiate transcription at levels detectable above background. Within the promoter sequence can be found a transcription initiation site (defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced (if the coding sequence contains introns) and translated into the protein encoded by the coding sequence.

“Transcriptional and translational control sequences” are nucleic acid regulatory sequences, such as promoters, enhancers, terminators, and the like, that provide for the expression of a coding sequence in a cell. For example, in eukaryotic cells, polyadenylation signals are control sequences.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., the coding sequence is under the transcriptional control of the promoter). Coding sequences can be operably linked to regulatory sequences in sense and/or antisense orientation.

The nucleic acids or plasmids or vectors may further comprise at least one promoter suitable for driving expression of a nucleotide sequence in a cell. The term “expression vector” means a vector, plasmid or vehicle designed to enable the expression of an inserted nucleotide sequence following delivery of a nucleotide sequence into a cell. The cloned nucleotide sequence, i.e., the inserted nucleotide sequence, is usually placed under the control of control elements such as a promoter, a minimal promoter, an enhancer, or the like. Initiation control regions or promoters, which are useful to drive expression of a nucleic acid in a cell are numerous and familiar to those skilled in the art. Virtually any promoter capable of driving expression of a nucleotide sequence is suitable for the present invention, including but not limited to, viral promoters, bacterial promoters, animal promoters, mammalian promoters, synthetic promoters, constitutive promoters, tissue specific promoters, developmental specific promoters, inducible promoters, and/or light regulated promoters.

Vectors may be introduced into the desired cells by methods known in the art, e.g., transfection, electroporation, microinjection, transduction, cell fusion, DEAE dextran, calcium phosphate precipitation, lipofection (lysosome fusion), use of a gene gun, and/or a nucleic acid vector transporter in any order and in any combination (see, e.g., Wu et al., J. Biol. Chem. 267:963 (1992); Wu et al., J. Biol. Chem. 263:14621 (1988); and Hartmut et al., Canadian Patent Application No. 2,012,311, filed Mar. 15, 1990).

In some embodiments, a polynucleotide or nucleic acid of this invention can be delivered to a cell in vivo by lipofection. Synthetic cationic lipids designed to limit the difficulties and dangers encountered with liposome-mediated transfection can be used to prepare liposomes for in vivo transfection of a nucleotide sequence of this invention (Feigner et al., Proc. Natl. Acad. Sci. USA 84:7413 (1987); Mackey, et al., Proc. Natl. Acad. Sci. U.S.A. 85:8027 (1988); and Ulmer et al., Science 259:1745 (1993)). The use of cationic lipids may promote encapsulation of negatively charged nucleic acids, and also promote fusion with negatively charged cell membranes (Feigner et al., Science 337:387 (1989)). Particularly useful lipid compounds and compositions for transfer of nucleic acids are described in International Patent Publications WO 95/18863 and WO 96/17823, and in U.S. Pat. No. 5,459,127. The use of lipofection to introduce exogenous nucleotide sequences into specific organs in vivo has certain practical advantages. Molecular targeting of liposomes to specific cells represents one area of benefit. It is clear that directing transfection to particular cell types would be particularly preferred in a tissue with cellular heterogeneity, such as bone marrow, pancreas, liver, kidney, and the brain. Lipids may be chemically coupled to other molecules for the purpose of targeting (Mackey, et al., 1988, supra). Targeted peptides, e.g., hormones or neurotransmitters, and proteins such as antibodies, or non-peptide molecules could be coupled to liposomes chemically.

In various embodiments, other molecules can be used for facilitating delivery of a nucleic acid in vivo, such as a cationic oligopeptide (e.g., as described in International Patent Publication No. WO 95/21931), peptides derived from nucleic acid binding proteins (e.g., as described in International Patent Publication No. WO 96/25508), and/or a cationic polymer (e.g., as described in International Patent Publication No. WO 95/21931).

It is also possible to deliver a nucleic acid of this invention to a subject in vivo as naked nucleic acid (see, e.g., U.S. Pat. Nos. 5,693,622, 5,589,466 and 5,580,859). Receptor-mediated nucleic acid delivery approaches can also be used (Curiel et al., Hum. Gene Ther. 3:147 (1992); Wu et al., J. Biol. Chem. 262:4429 (1987)).

The term “transfection” means the uptake of exogenous or heterologous nucleic acid (RNA and/or DNA) by a cell. An “exogenous nucleotide sequence,” “heterologous nucleotide sequence” or “exogenous or heterologous nucleic acid” is typically a nucleotide sequence or nucleic acid molecule that is not naturally occurring in the virus genome in which it is present and/or is not naturally occurring in the cell into which it is introduced or is not naturally occurring in the cell into which it is introduced in the form and/or amount in which it is present in the cell upon introduction. Generally, the heterologous nucleic acid or nucleotide sequence comprises an open reading frame that encodes a peptide, a polypeptide and/or a nontranslated functional RNA.

A cell has been “transfected” with an exogenous or heterologous nucleic acid when such nucleic acid has been introduced or delivered inside the cell. A cell has been “transformed” by exogenous or heterologous nucleic acid when the transfected nucleic acid imparts a phenotypic change in the cell and/or in an activity or function of the cell. The transforming nucleic acid can be integrated (covalently linked) into chromosomal DNA making up the genome of the cell and/or it can be present as a plasmid (e.g., stably integrated and/or transient).

As used herein, “transduction” of a cell means the transfer of genetic material into the cell by the incorporation of nucleic acid into a virus particle and subsequent transfer into the cell via infection of the cell by the virus particle.

As used herein, the term “polypeptide” encompasses both peptides and proteins, unless indicated otherwise.

The terms “polypeptide,” “protein,” and “peptide” refer to a chain of covalently linked amino acids. In general, the term “peptide” refers to shorter chains of amino acids (e.g., 2-50 amino acids); however, all three terms overlap with respect to the length of the amino acid chain. Polypeptides, proteins and peptides may comprise naturally occurring amino acids, non-naturally occurring amino acids, or a combination of both. The polypeptides, proteins and peptides may be isolated from sources (e.g., cells or tissues) in which they naturally occur, produced recombinantly in cells in vivo or in vitro or in a test tube in vitro, and/or synthesized chemically. Such techniques are known to those skilled in the art. See, e.g., Sambrook et al., Molecular Cloning: A Laboratory Manual 2nd Ed. (Cold Spring Harbor, N.Y., 1989); Ausubel et al. Current Protocols in Molecular Biology (Green Publishing Associates, Inc. and John Wiley & Sons, Inc., New York).

The term “fragment,” as applied to a polypeptide or protein of this invention, will be understood to mean an amino acid sequence of reduced length relative to a reference (e.g., full length or “wild type”) polypeptide or amino acid sequence and comprising, consisting essentially of, and/or consisting of an amino acid sequence of contiguous amino acids identical to or substantially similar to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, 250, 300, 350, 400, 450, 500, 550, 600, 650, 700, 750, 800, 850 or 900 or more consecutive amino acids of a polypeptide or amino acid sequence according to the invention.

As used herein, “fragment” also refers to a portion of a KSHV Orf63 protein that retains at least one biological activity associated with KSHV Orf63 (e.g., reduction of inflammation) and can have at least about 50% 60%, 65%, 70%, 75%, 80%, 85%, 90% 95% or more of the biological activity as compared with the full-length (e.g., reference) protein or even has a greater level of biological activity.

The term “domain” as used herein is intended to encompass a part of a protein sequence and structure that can evolve, function and exist independently of the rest of the protein chain. A domain is capable of forming a compact three-dimensional structure and often can be independently stable and folded. One domain may appear in a variety of evolutionarily related proteins. Domains can vary in length from between about 25 amino acids up to about 500, 600, 700 or 800 amino acids in length. A “domain” can also encompass a domain from a wild-type protein that has had an amino acid residue, or residues, replaced by conservative substitution. Because they are self-stable in a protein milieu, domains can be “swapped” by genetic engineering between one protein and another to make chimeric proteins.

The terms “variant” or “variants,” as used herein, are intended to designate KSHV Orf63 having the “wild type” or “parent” amino acid sequence (e.g., as provided under the GenBank® Database Accession number provided herein), wherein one or more amino acids of the parent sequence have been substituted by another amino acid and/or wherein one or more amino acids of the parent sequence have been deleted and/or wherein one or more amino acids have been inserted in the parent sequence protein and/or wherein one or more amino acids have been added to the parent sequence. Such addition can take place either at the N-terminal end or at the C-terminal end of the parent protein or both and/or in the interior of the sequence. The “variant” or “variants” within this definition still have KSHV Orf63 activity in their activated form. In one embodiment, a variant is at least 70% identical with the wild type or parent amino acid sequence of KSHV Orf63. In some embodiments a variant is at least 50%, 60%, 70%, 75%, 80%, 85, 90%, or 95% identical with the amino acid sequence of KSHV Orf63. In other embodiments a variant is at least 90% identical with the amino acid sequence of KSHV Orf63. In a further embodiment a variant is at least 95%, 96%, 97%, 98%, or 99% identical with the amino acid sequence of KSHV Orf63.

The variant may have “conservative” changes, wherein a substituted amino acid has similar structural or chemical properties. In particular, such changes can be guided by known similarities between amino acids in physical features such as charge density, hydrophobicity/hydrophilicity, size and configuration, so that amino acids are substituted with other amino acids having essentially the same functional properties. For example: Ala may be replaced with Val or Ser; Val may be replaced with Ala, Leu, Met, or Ile, preferably Ala or Leu; Leu may be replaced with Ala, Val or Ile, preferably Val or Ile; Gly may be replaced with Pro or Cys, preferably Pro; Pro may be replaced with Gly, Cys, Ser, or Met, preferably Gly, Cys, or Ser; Cys may be replaced with Gly, Pro, Ser, or Met, preferably Pro or Met; Met may be replaced with Pro or Cys, preferably Cys; His may be replaced with Phe or Gln, preferably Phe; Phe may be replaced with His, Tyr, or Trp, preferably H is or Tyr; Tyr may be replaced with His, Phe or Trp, preferably Phe or Trp; Trp may be replaced with Phe or Tyr, preferably Tyr; Asn may be replaced with Gln or Ser, preferably Gln; Gln may be replaced with His, Lys, Glu, Asn, or Ser, preferably Asn or Ser; Ser may be replaced with Gln, Thr, Pro, Cys or Ala; Thr may be replaced with Gln or Ser, preferably Ser; Lys may be replaced with Gln or Arg; Arg may be replaced with Lys, Asp or Glu, preferably Lys or Asp; Asp may be replaced with Lys, Arg, or Glu, preferably Arg or Glu; and Glu may be replaced with Arg or Asp, preferably Asp. Once made, changes can be routinely screened to determine their effects on function.

Alternatively, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, such as for example, LASERGENE™ software. In particular embodiments, a “functional variant” retains at least one biological activity normally associated with KSHV Orf63. In particular embodiments, the “functional variant” retains at least about 40%, 50%, 60%, 75%, 85%, 90%, 95%, 97%, 98% or more biological activity normally associated with KSHV Orf63.

As used herein, “derivative” refers to a component that has been subjected to a chemical modification. For example, derivatization of a protein component can involve the replacement of a hydrogen by an acetyl, acyl, alkyl, amino, formyl, or morpholino group. Derivative molecules can retain the biological activities of the naturally occurring molecules but can confer advantages such as longer lifespan and/or enhanced activity.

In particular embodiments, a biologically active variant or derivative of any of the protein components of this invention has at least about 50%, 60%, 65%, 70%, 75%, 80%, 85%, 90%, 95%, 96%, 97%, 98%, 99% or more amino acid sequence similarity or identity with the amino acid sequence of a naturally-occurring protein.

A domain or fragment of a polypeptide or protein of this invention can be produced by methods well known and routine in the art. Fragments of this invention can be produced, for example, by enzymatic or other cleavage of naturally occurring peptides or polypeptides or by synthetic protocols that are well known. Such fragments can be tested for one or more of the biological activities of this invention (e.g., reducing inflammation and/or promoting and/or accelerating healing of a wound, ulcer, incision site, etc.) according to the methods described herein, which are routine methods for testing activities of polypeptides and/or peptides, and/or according to any art-known and routine methods for identifying such activities. Such production and testing to identify biologically active fragments and peptides of the polypeptides described herein would be well within the scope of one of ordinary skill in the art and would be routine.

The invention further provides homologues, as well as methods of obtaining homologues, of the polypeptides and/or fragments and/or peptides of this invention from other organisms. As used herein, an amino acid sequence or protein is defined as a homologue of a polypeptide or fragment or peptide of the present invention if it shares significant homology or identity to a polypeptide, peptide and/or fragment of the present invention. Significant homology or identity means at least 50%, 60%. 65%, 70-%, 75%, 80%, 85%, 90%, 95%, 97%, 98%, 99% and/or 100% homology or identity with another amino acid sequence. In some embodiments, by using the nucleic acids that encode the KSHV Orf63 proteins, peptides and/or fragments of this invention (as are known in the art and incorporated by reference herein), as a probe or primer, and techniques such as PCR amplification and colony/plaque hybridization, one skilled in the art can identify homologues of the KSHV Orf63 polypeptides, peptides and/or fragments of this invention in other organisms on the basis of information available in the art.

A subject of this invention is any subject that is susceptible to inflammation and/or inflammatory diseases and disorders. Nonlimiting examples of a subject of this invention include mammals, such as humans, nonhuman primates, domesticated mammals (e.g., dogs, cats, rabbits), laboratory animals (e.g., mice, rats and other rodents), livestock and agricultural mammals (e.g., horses, cows, pigs).

A subject of this invention can be “in need of” the methods of the present invention, e.g., because the subject has, or is believed at risk for, a disorder including those described herein and/or is a subject that would benefit from the methods of this invention. For example, a subject in need of the methods of this invention can be, but is not limited to, a subject diagnosed with, having or suspected to have, or at risk of having or developing inflammation and/or an inflammatory disease or disorder.

The term “percent identity,” as known in the art, describes a relationship between two or more polypeptide sequences or two or more polynucleotide sequences, as determined by comparing the sequences. In the art, “identity” also means the degree of sequence relatedness between polypeptide or polynucleotide sequences as determined by the match between strings of such sequences. “Identity” and “similarity” can be readily calculated by known methods, including but not limited to those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

Exemplary methods to determine identity are designed to give the best match between the sequences tested. Methods to determine identity and similarity are codified in publicly available computer programs. Sequence alignments and percent identity calculations can be performed using the Megalign program of the LASERGENE bioinformatics computing suite (DNASTAR Inc., Madison, Wis.). Multiple alignments of sequences may be performed using the Clustal method of alignment (Higgins and Sharp (1989) CABIOS 5:151-153), with the default parameters (GAP PENALTY=10, GAP LENGTH PENALTY=10). Exemplary default parameters for pairwise alignments using the Clustal method can be selected: KTUPLE 1, GAP PENALTY=3, WINDOW=5 and DIAGONALS SAVED=5.

The term “sequence analysis software” refers to any computer algorithm or software program that is useful for the analysis of nucleotide and/or amino acid sequences. “Sequence analysis software” is commercially available or can be independently developed. Typical sequence analysis software will include but is not limited to the GCG suite of programs (Wisconsin Package Version 9.0, Genetics Computer Group (GCG), Madison, Wis.), BLASTP, BLASTN, BLASTX (Altschul et al., J. Mol. Biol. 215:403-410 (1990), and DNASTAR (DNASTAR, Inc. 1228 S. Park St. Madison, Wis. 53715 USA). Within the context of this application it will be understood that where sequence analysis software is used for analysis, the results of the analysis will be based on the “default values” of the program referenced, unless otherwise specified. As used herein “default values” will mean any set of values or parameters, which originally load with the software when first initialized.

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

The alignment may include the introduction of gaps in the sequences to be aligned. In addition, for sequences which contain either more or fewer amino acids than the polypeptides specifically disclosed herein, it is understood that in one embodiment, the percentage of sequence identity will be determined based on the number of identical amino acids in relation to the total number of amino acids. Thus, for example, sequence identity of sequences shorter than a sequence specifically disclosed herein, will be determined using the number of amino acids in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

A “therapeutic polypeptide,” “therapeutic peptide” or “therapeutic fragment” is a polypeptide, peptide or fragment that can alleviate or reduce symptoms that result from an absence or defect or deficiency in a protein in a cell or subject. Alternatively, a “therapeutic polypeptide,” “therapeutic peptide” or “therapeutic fragment” is a polypeptide, peptide or fragment that otherwise confers a benefit to a subject, e.g., by reducing inflammation and/or alleviating or reducing the symptoms of an inflammatory disease or disorder.

The term “therapeutically effective amount” or “effective amount,” as used herein, refers to that amount of a polypeptide, peptide, fragment, nucleic acid, virus and/or composition of this invention that imparts a modulating effect, which, for example, can be a beneficial effect, to a subject afflicted with a condition (e.g., a disorder, disease, syndrome, illness, injury, traumatic and/or surgical wound), including improvement in the condition of the subject (e.g., in one or more symptoms), delay or reduction in the progression of the condition, prevention or delay of the onset of the condition, and/or change in clinical parameters, status or classification of a disease or illness, etc., as would be well known in the art.

For example, a therapeutically effective amount or effective amount can refer to the amount of a polypeptide, peptide, fragment, nucleic acid, virus, composition, compound and/or agent that improves a condition (e.g., reduces inflammation or ameliorates the symptoms of an inflammatory disease or disorder) in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to a control or reference.

“Treat” or “treating” or “treatment” refers to any type of action that imparts a modulating effect, which, for example, can be a beneficial effect, to a subject afflicted with a condition (e.g., disorder, disease, syndrome, illness, traumatic or surgical wound, injury, etc.), including improvement in the condition of the subject (e.g., in one or more symptoms), delay or reduction in the progression of the condition, prevention or delay of the onset of the condition, and/or change in clinical parameters, disease or illness, etc., as would be well known in the art.

By the terms “treat,” “treating” or “treatment of” (or grammatically equivalent terms), it is also meant that the severity of the subject's condition is reduced or at least partially improved or ameliorated and/or that some alleviation, mitigation or decrease in at least one clinical symptom is achieved and/or there is a delay in the progression of the condition and/or prevention or delay of the onset of a disease or disorder. In certain embodiments, the methods of this invention can be employed to reduce inflammation and/or reduce or ameliorate the symptoms of an inflammatory disease or disorder.

By “prevent,” “preventing” or “prevention” is meant to avoid or eliminate the development and/or manifestation of a pathological state and/or disease condition or status in a subject.

In methods of this invention, the disorder can be any disorder in which treatment of infection and/or reduction of inflammation (e.g., at a lesion site and/or wound site and/or disease site and/or surgical site) in a subject is indicated and/or desired. In some embodiments of this invention, the disorder can be, but is not limited to, diabetic ulcer, periodontal disease, chronic lesions, wounds, and any combination thereof.

In the methods of this invention in which inflammation is reduced, the inflammation can be reduced (e.g., reduced by 10%, 20%, 30%, 40%, 50%, 60%, 70%, 80%, 90%, 95% or 100%) as compared to the amount of inflammation present at a lesion and/or wound site prior to contact with agent of this invention. In particular embodiments, the amount of inflammation can be substantially reduced (e.g., by at least about 30%, 40%, 50%, 60%, 70%, 80%, 90%, 100%). Thus in some embodiments of this invention, the site of inflammation is contacted with the agent of this invention for a period of time sufficient to reduce inflammation by at least about 50% (i.e., to substantially reduce inflammation).

The amount of inflammation can be determined by measuring the amount of pro-inflammatory agents (e.g., IL-6, IL-1beta, IL-18, MMP-1, etc.) present at the site of inflammation and/or in a subject according to protocols as described herein and as are well known in the art. A reduction in the amount of inflammation is also determined by measuring the amount of pro-inflammatory agents at the site of inflammation before and after contact with an agent (e.g., polypeptide, fragment, peptide, nucleic acid vector, etc. of this invention) of this invention. A reduction in the amount of the pro-inflammatory agents under analysis indicates a reduction in inflammation and the amount of inflammation reduction as measured by percent can be determined from such assays according to methods standard in the art.

In particular embodiments, the present invention provides a composition comprising, consisting essentially of and/or consisting of a protein, peptide, fragment nucleic acid and/or virus of this invention in a pharmaceutically acceptable carrier and, optionally, further comprising other medicinal agents, pharmaceutical agents, stabilizing agents, buffers, carriers, adjuvants, diluents, etc.

In some embodiments, a composition of this invention can comprise, consist essentially of and/or consist of a protein, peptide, fragment, nucleic acid and/or virus of this invention in combination with an anti-inflammatory agent, a cytokine, an immune modulator and/or a locally acting analgesic (e.g., lidocaine). In some embodiments, a composition of this invention can comprise, consist essentially of and/or consist of a protein, peptide, fragment, nucleic acid and/or virus of this invention in combination with a nucleic acid encoding an anti-inflammatory agent and/or cytokine of this invention.

Further provided herein is a pharmaceutical composition comprising a protein, peptide, fragment, nucleic acid and/or virus of this invention in a pharmaceutically acceptable carrier, in any combination.

“Pharmaceutically acceptable,” as used herein, means a material that is not biologically or otherwise undesirable, i.e., the material may be administered to a subject along with the compositions of this invention, without causing substantial deleterious biological effects or interacting in a deleterious manner with any of the other components of the composition in which it is contained. The material would naturally be selected to minimize any degradation of the active ingredient and to minimize any adverse side effects in the subject, as would be well known to one of skill in the art (see, e.g., Remington's Pharmaceutical Science; latest edition). Exemplary pharmaceutically acceptable carriers for the compositions of this invention include, but are not limited to, sterile pyrogen-free water and sterile pyrogen-free physiological saline solution, as well as other carriers suitable for injection into and/or delivery to a subject of this invention, particularly a human subject, as would be well known in the art.

A further aspect of the invention is a method of administering or delivering a KSHV Orf63 protein, peptide, fragment, nucleic acid and/or virus of the invention to a subject of this invention. Administration or delivery to a human subject or an animal in need thereof can be by any means known in the art for administering proteins, peptides, fragments, nucleic acids and/or viruses. In some embodiments, the protein, peptide, fragment, nucleic acid and/or virus is delivered in a therapeutically effective dose in a pharmaceutically acceptable carrier.

In embodiments in which a nucleic acid of this invention is delivered in a viral vector (e.g., a virus particle), the dosage of virus particles to be administered to a subject will depend upon the mode of administration, the disease or condition to be treated, the individual subject's condition, the particular virus vector, and the nucleic acid to be delivered, and can be determined in a routine manner. Exemplary doses are virus titers of at least about 10⁵, 10⁶, 10⁷, 10⁸, 10⁹, 10¹⁰, 10¹¹, 10¹², 10³, 10¹⁴, 10¹⁵ transducing units or more, including in some embodiments about 10⁸-10¹³ transducing units and including in yet other embodiments about 10¹² transducing units.

In some embodiments, more than one administration (e.g., two, three, four or more administrations) of the protein, peptide, fragment, nucleic acid and/or viral vector may be employed to achieve the desired level of gene expression over a period of various intervals, e.g., daily, weekly, monthly, yearly, etc.

Exemplary modes of administration of the proteins, peptides, fragments, nucleic acids and/or vectors of this invention can include oral, rectal, transmucosal, topical, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration, and the like, as well as direct tissue or organ injection (e.g., to liver, skeletal muscle, cardiac muscle, diaphragm muscle or brain). Administration can also be to a tumor (e.g., in or a near a tumor or a lymph node). The most suitable route in any given case will depend on the nature and severity of the condition being treated and on the nature of the particular protein, peptide, fragment, nucleic acid and/or vector that is being used.

For injection, the carrier will typically be a liquid. For other methods of administration, the carrier may be either solid or liquid. For inhalation administration, the carrier will be respirable, and will preferably be in solid or liquid particulate form.

As described in the embodiments herein, a protein, peptide, fragment, nucleic acid and/or vector a can be administered directly to an injury and/or trauma and/or surgical site of a subject according to the methods of this invention as described herein. In certain embodiments, the protein, peptide, fragment, nucleic acid and/or virus vector will be present in a pharmaceutical composition further comprising a pharmaceutically acceptable carrier.

Dosages of the KSHV Orf63 protein, peptides and/or active fragment thereof to be administered to a subject will depend upon the mode of administration, the disease or condition to be treated, the individual subject's condition, the particular protein, peptide and/or fragment or nucleic acid encoding same, and any other agents being administered to the subject and can be determined in a routine manner according to methods well known in the art. An exemplary dosage range for a human subject is from about 1 microgram/ml of vehicle to about 500 milligrams/ml of vehicle

In particular embodiments, more than one administration (e.g., two, three, four or more administrations) of the protein, peptide, fragment and/or nucleic acid of this invention may be employed to achieve the desired result over a period of various intervals, e.g., daily, weekly, monthly, yearly, etc.

The present invention will now be described with reference to the following examples. It should be appreciated that these examples are for the purposes of illustrating aspects of the present invention, and do not limit the scope of the invention as defined by the claims.

### EXAMPLES

Bioinformatics: NCBI BLASTP for NLRP1 and Orf63 was performed using the amino acid sequence of NLRP1 (NCBI accession # NP_{—}127499) and the amino acid sequence of KSHV Orf63 (NCBI accession# YP_{—}001129420.1). The ClustalW2 program was used to align the full-length sequence of NLRP1 isoform 1 (NCBI accession # NP_{—}127497), NLRP1 isoform 3 (NCBI accession # NP_{—}127499.1), NOD2 (NCBI accession # Q9HC29), KSHV Orf63 (NCBI accession # YP 001129420.1), and RRV Orf63 (NCBI accession # AAF60049).

Reagents: Total RNA was harvested from THP-1 cells with TriZol and, cDNA synthesis was performed using Superscript III reverse transcriptase. PCR reactions were performed using Pfx DNA polymerase with primers designed to the open reading frames of procaspase-1, pro-IL-1beta, NOD1, NOD2, and NLRP3. The amplicons were cloned into pcDNA3.1D to make V5 tagged pcDNA3-procaspasel-V5, pcDNA3-pro-IL-1β-V5, and pcDNA3-NOD2-V5 plasmids. Epitope tags were interchanged using annealed oligonucleotides for FLAG (pcDNA3-ASC-FLAG, pcDNA3-NOD1-FLAG) or Myc (pcDNA3-NLRP3-myc) using standard procedures. Sequence fidelity was verified by DNA sequencing. Expression was verified by immunoblot analysis to epitope tags of transfected plasmids. pUNO-NLRP1 was purchased from Invivogen. pcDNA3-NLRP1-V5 was generated by amplifying isoform 1 of NLRP1 by PCR and subcloning into pcDNA3. KSHV Orf63 was amplified by PCR using primers adding BamHI and EcoRV restriction sites with a FLAG epitope tag at the c-terminus. Amplified Orf63 was cloned into pcDNA3 vector to generate pcDNA3-Orf63-FLAG. The same procedure was used to generate pcDNA3-Orf63-V5. Orf63-N-FLAG and Orf63ΔN-FLAG were generated by cloning fragments encoding amino acids 1-200 (Orf63-N) and 201-928 (Orf63ΔN). To generate GST-tagged Orf63, Orf63 was subcloned from pcDNA3-Orf63-FLAG using BamHI and NotI restriction sites and cloned into pGex5p-1 vector. pcDNA-NLRP1-myc, accompanying myc-tagged mutants and GST-tagged NLRP1 were previously described (15).

Cell lines: BCBL-1, THP-1-Orf63 and THP-1-Control cells were maintained in RPMI medium 1640 (Cellgro) containing 10% FBS and 1% penicillin-streptomycin (PS). 293T and lentivirus producer 293FT cells were maintained in DMEM medium (Cellgro) with 10% FBS and PS. KSHV-293T were maintained as 293T with 1 mg/ml Puromycin. Cells were maintained at 37° C. in 5% carbon dioxide. BCBL-1-shRNA-NLRP1 and BCBL-1-shRNA-non-targeting cell lines were generated by nucleofecting BCBL-1 cells with shRNA-NLRP1 or shRNA-non-targeting (Santa Cruz) control plasmids. Nucleofected cells were selected for expression of smRNA plasmids by the addition of 1.0 ug/ml puromycin. Knock-down of NLRP1 was confirmed by immunoblotting for NLRP1 (Cell Signaling).

NLRP1 Inflammasome Reconstitution Assays: 0.2×10⁶ HEK293T cells were seeded in 24-well plates in cell culture media lacking antibiotics. 24 hours later, cells were transfected with 200 ng pUNO-NLRP1 (Invitrogen), 5 ng ASC-FLAG, 5 ng pro-caspase-1-V5 and either 5 ng human pro-IL-1β-V5 or 20 ng mouse pro-IL-1β (14) using lipofectamine-2000 (Invitrogen). 24 hours later, cell lysates and supernatants were harvested and analyzed for IL-1β secretion and expression by ELISA (BD) or immunoblot, respectively. Secreted IL-1β levels were generally normalized by using a LDH release assay to normalize for cell death. For immunoblotting, cleaved IL-1β (Cell signaling) or murine IL-1β (R&D Systems) antibodies were used to detect human cleaved- or mouse pro- and cleaved-IL-1β expression, respectively. Immunoblots of extracellular IL-1β were performed after precipitation with trichloracetic acid.

Orf63-Lentivirus Production and Infection: Orf63-FLAG was subcloned into a pLenti7.3 lentivirus expression system (Invitrogen) according to manufacturer's instructions. Briefly, Orf63-FLAG was PCR amplified to include a 5′-terminus KOZAK sequence and subcloned into pENTR Gateway vector to yield pENTR-Orf63-FLAG (Note: stop codon not mutated to enable FLAG epitope detection). Next, pLenti7.3-Orf63-FLAG was generated by performing LR recombination reaction between pENTR-Orf63-FLAG and pLenti7.3 expression vector. A clone containing pLenti7.3 that did not recombine was isolated as a negative control. Orf63 expression was confirmed by transfecting into 293T cells and performing anti-FLAG immunoblots. To generate lentivirus, pLenti7.3-Orf63-FLAG or control pLenti7.3 were transfected into 293FT cells (Invitrogen) with ViraPower Packaging Mix. Virus was harvested 72 hours later. Transduction of THP-1 cells was performed by centrifugation of 5×10⁶ cells with 1 ml virus, 8 ug/ml polybrene for 3 hours at 3000 rpm at room temperature (RT). Twenty-four hours later, the transduction was repeated to enhance infection. The transduced cells were sorted for GFP expression by flow cytometry and Orf63 expressing clones isolated by limiting dilution. Expression of FLAG-tagged Orf63 in THP-1-Orf63 cells was confirmed by immunoblot.

THP-1 Stimulations: 1×10⁶ pLenti7.3 or pLenti7.3-Orf63 cells were plated in 12-well plates in 500 ul RPMI containing 2% FBS and PS. Cells were either mock treated or primed with 5 ng/ml lipopolysaccharide (Sigma) for 1 hour followed by treatment with 10 μg/ml muramyldipeptide (Sigma), 2.5 mM adenosine triphosphate (ATP), or 130 μg/ml Alum (Thermo Scientific) for 6 hours at 37° C. Supernatants were harvested and clarified by centrifugation at 1500 rpm at RT and cytokine analysis was performed as described below.

Immunoblot and Co-immunoprecipitation Assays: Cell lysates were prepared in 0.1% NP-40 lysis buffer containing protease inhibitor cocktail (Roche). Proteins were resolved on either 10% or 12.5% SDS-PAGE gels and transferred to PVDF membranes by wet-transfer. Membranes were blocked for 1 hour in 5% non-fat dry milk (NFDM) containing 0.1% TBS-Tween 20 and washed 3× in 0.1% TBS-Tween 20 at room temperature. Monoclonal, horse-radish peroxidase (HRP) conjugated antibodies (Sigma) to M2-FLAG, C-myc, and V5 epitopes were used to detect expression of epitope tagged proteins. IL-1β and cleaved IL-1β antibodies (Cell signaling) were used at 1:1000 in 5% bovine serum albumin overnight followed by anti-rabbit-HRP secondary antibody (Cell Signaling) to detect the 34 kDa and 17 kDa forms of IL-1β. Detection of cleaved IL-1β in supernatants was performed by concentrating supernatants using Millipore YP-10 centrifugal filter devices or trichloroacetic acid precipitation followed by immunoblot. For co-immunoprecipitation studies, antibodies against the FLAG, V5 or c-myc epitopes were used to precipitate proteins in the presence of 20 μl protein A/G beads (Santa Cruz) overnight at 4° C. Protein complexes were washed four times in either lysis or RIPA buffer, incubated at 95° C. for 5 minutes and resolved by immunoblot. To detect Orf63's interaction with endogenous NLRP1, Orf63 was immunoprecipitated from THP-1 cells 24 hours following nucleofection (Amaxa) using anti-V5 monoclonal antibody (Sigma) as described herein. Endogenous NLRP1 was detected by immunoblotting using human anti-NLRP1 antibody (Cell Signaling). For direct immunoprecipitation, Orf63 was expressed in BL21 Escherichia coli cells by induction with 1 mM isopropyl β-D-1-thiogalactopyranoside (IPTG) for 2 hours at 37° C. GST-Orf63 was purified with GST-sepharose (Amersham). GST-sepharose-Orf63 was incubated with precision protease overnight at 4° C. Cleaved Orf63 was further purified by incubation with anti-FLAG-M2 antibody for 1 hour at 4° C. followed by incubation with protein A/G beads for 3 hours. Next, beads were washed 5 times with lysis buffer to remove unbound Orf63 and resuspended in 1 ml lysis buffer and purified NLRP1. Mouse IgG (Santa Cruz) control reactions were also prepared. Orf63 and NLRP 1 were incubated overnight at 4° C., washed 4 times with lysis buffer and NLRP1 interactions were detected by immunoblotting for NLRP 1.

In-vitro transcription/translation: [³⁵S]-methionine (Perkin-Elmer) labeled NLRP1, NLRP3 and control luciferase proteins were prepared using TnT T7 Quick Coupled Transcription/Translation System (Promega) according to manufacturer's instructions. pcDNA3-NLRP1 and pcDNA3-NLRP3 plasmids were used for the transcription/translation reaction.

GST binding Assay: Expression and purification of GST fusion proteins were performed as previously described (29) and equal amounts of GST and GST-Orf63 were bound to glutathione beads (29). Equivalent amounts (1×10⁵ cpm) of ³⁵S-methionine labeled NLRP1, NLRP3 and luciferase proteins were incubated with the GST and GST-Orf63 bound protein in NETN+ buffer plus protease inhibitors for an hour at 4° C. NETN+ buffer comprised of 20 mM Tris (pH 7.5), 100 mM NaCl, 1 mM EDTA, 0.1% NP-40, 1 mM DTT. The bound complexes were washed five times (5 minute washes) in NETN+ buffer. The samples were subjected to SDS-PAGE. The gel was fixed, dried and exposed to a phosphoimager.

Gel Filtration Assay: HEK293T cells were plated in 15 cm dishes in antibiotic-free media. 24 hours later, cells were transfected with NLRP1-myc or NLRP1-myc and Orf63-flag expression plasmids with lipofectamine-2000 as described. 24 hours post-transfection, cells were washed with ice-cold 1×PBS followed by lysis in hypotonic buffer (20 mM HEPES-KOH pH7.5, 10 mM KCL, 1.5 mM MgCl₂, 1 mM EDTA, 1 mM EGTA and Roche Protease Inhibitor Cocktail) and shearing with a 27-gauge needle. Soluble lysates were run over a Superose 6 gel filtration column (GE Lifesciences) using a Bio-Rad Duoflow chromatography system. Column elution fractions were analyzed for comigration of NLRP 1 and Orf63 by immunoblot for myc and FLAG, respectively.

Caspase-1 Fluorescence Assay: Caspase-1 fluorescence assay (R&D systems) was performed according to manufacturer's instructions. Briefly, 293T cells were transfected as described herein. Twenty-four hours later, cells were washed in ice-cold 1×PBS, and whole cell extracts prepared. 50 μL of extract was added to 50 mL of 2× reaction buffer containing DTT, followed by 5 μL, of WEHD-7-amino-4-trifluoromethyl coumarin (AFC) substrate. Caspase-1 activity was measured every 15 minutes over 2 hours using a fluorescence plate reader at 505 nm.

ELISA: Supernatants were analyzed for cytokine expression using IL-1β, TNF-α (BD Biosciences) and IL-18 (R&D Systems) ELISAs according to the manufacturer's instructions. IL-1β secretion was normalized by the LDH release assay.

Lactate-Dehydrogenase Release Assay: LDH release assays (Promega) were performed according to the manufacturer's instructions. LDH release data were used to normalize IL-1β secretion in 293T inflammasome reconstitution and THP-1 stimulation assays to account for cell death.

Reactivation Assays: KSHV-293T cells were seeded at 0.5×10⁶ cells per well in a 6-well plate. Cells were transfected 24 hours later with plasmids encoding procaspase-1, ASC, pro-IL-1β, RTA or vector control, along with either 50 nM Orf630N-TARGET (Dharmacon) siRNA (Sense: AGACAAAGCUGUUGAUGGAUU, Antisense: UCCAUCAACAGCUUUGUCUUU) or control non-targeting siRNA (Dharmacon). Cells and supernatants were harvested forty eight hours later. Supernatants were subjected to IL-1β ELISA while total RNA was isolated from the cell pellets. In order to confirm knockdown of Orf63, cDNA was generated and used as a template for PCR with Orf63 primers (Sense: CCCACTACGCGGATCAGATA, Antisense: GCTCTTGCATAATGCCTCTA). GAPDH primers were used for loading control. PCR products were resolved on a 1% agarose gel. NLRP1 inhibition of reactivation was performed by coexpressing NLRP1 inflammasome components as described herein with RTA or vector control. vIL-6 expression was analyzed by immunoblot 72 hours post-reactivation.

Inhibition of KSHV reactivation in BCBL-1. 1×10⁶BCBL-1-shRNA-NLRP1 or BCBL-1-shRNA-control cells were either mock treated or treated with 25 ng/ml TPA (Sigma). Viral genomes were analyzed by qPCR using primers specific for Orf49 as previously described 96 hours post-reactivation (6).

Analysis of KSHV transcription: 2.5×10⁶ BCBL-1 cells were nucleofected with siRNAs against Orf63 or non-targeting control. 24 hours later, cells were either mock stimulated or treated with 25 ng/ml TPA for 96 hours. Orf49 and Orf57 viral gene transcription was analyzed as previously described (6).

Human Primary Monocyte Infections: KSHV was grown and purified as previously described (7). Primary human monocytes were isolated from peripheral blood mononuclear cells by negative selection using the monocyte isolation kit II from Milltenyi Biotec. Purification of monocytes was verified by flow cytometry for CD14 (Miltenyi Biotec) and average purities were greater than 98%. Immediately following purification, 1.5×10⁷ monocyteswere infected with KSHV by centrifugation at 2000 rpm at 30° C. for 1 hour. KSHV-infected monocytes were cultured in 20% FBS. 48 hours post-infection, media was changed to antibiotic free media. 72 hours post-infection, productive infection was confirmed by fluorescence microscopy. Next, siRNAs against Orf63 or non-targeting control were transfected by Lipofectamine RNAimax (Invitrogen) according to manufacturer's instruction. 120 hours post-infection, supernatants were analyzed for IL-1β expression by ELISA and knockdown of Orf63 tested by PCR, qPCR for Orf49, Orf50 and Orf57 transcription was assessed as previously described (6).

Results: This is the first report of molecular mimicry of a NLR protein by a human virus and we show that KSHV Orf63, a viral homolog of human NLRP1, blocks NLR-dependent innate immune responses, including caspase-1 activation and processing of IL-1β and IL-18.

The nucleotide-binding and oligomerization, leucine-rich repeat (NLR) family of proteins sense microbial infections and activate the inflammasome, a multi-protein complex that promotes microbial clearance. Kaposi's sarcoma-associated herpesvirus (KSHV) is linked to several human malignancies. We report that KSHV Orf63 is a viral homolog of human NLRP1. Orf63 blocked NLRP1-dependent innate immune responses, including caspase-1 activation and processing of interleukin (IL)-1β and IL-18. KSHV Orf63 interacted with NLRP1, NLRP3, and NOD2. Inhibition of Orf63 expression resulted in increased expression of IL-1β during the KSHV lifecycle. Furthermore, inhibition of NLRP1 was necessary for efficient reactivation and generation of progeny virus. The viral homolog subverts the function of cellular NLRs, which indicates that modulation of NLR-mediated innate immunity is important for the life-long persistence of herpesviruses.

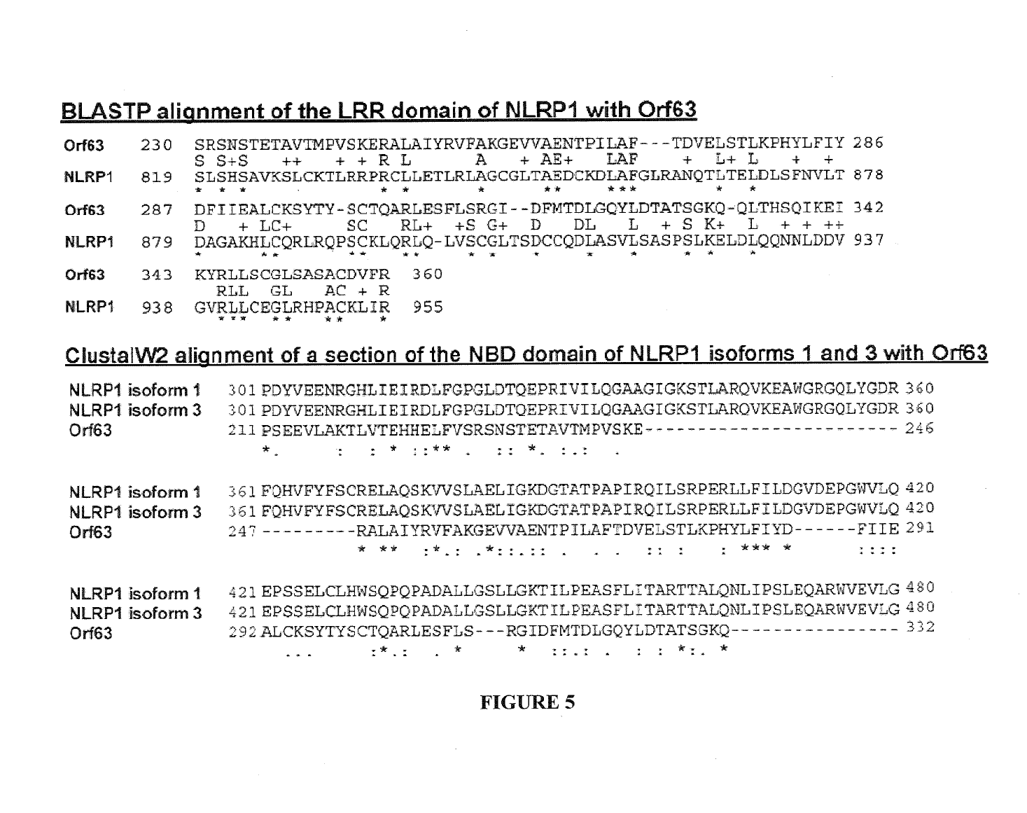

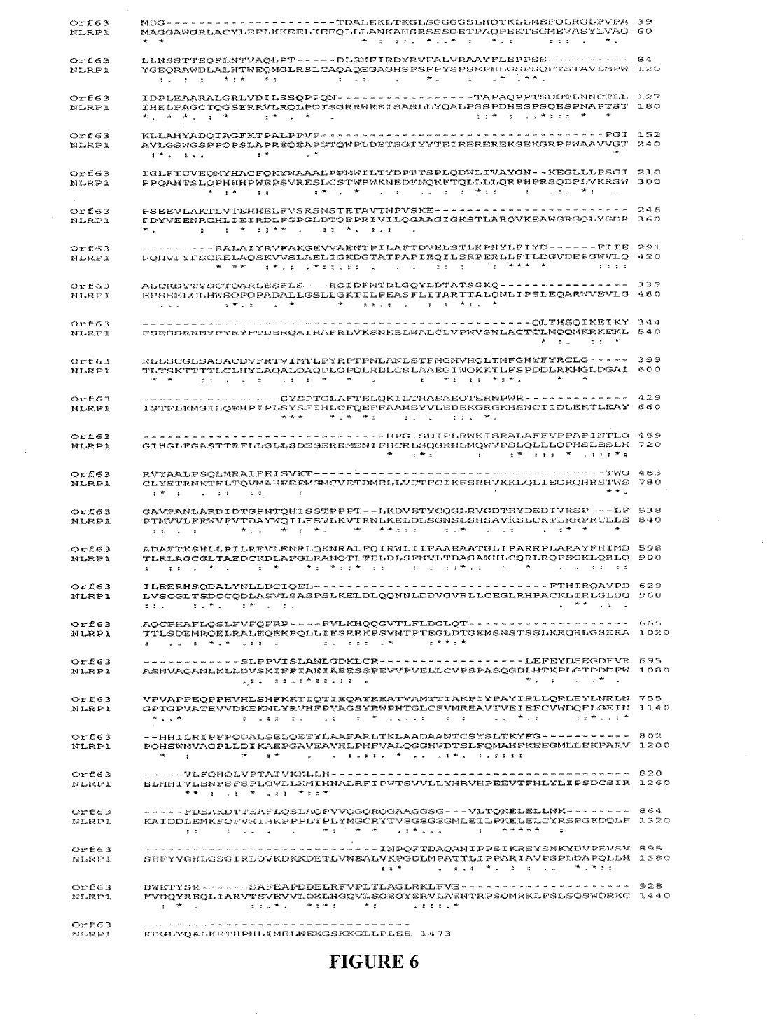

KSHV Orf63 is an uncharacterized tegument protein. Basic Local Alignment Search Tool for proteins (BLASTP) of NLRP1 and KSHV Orf63 revealed that these proteins are homologous (E value=0.0002) and that Orf63 showed significant similarity to the LRR domain of NLRP1 (FIG. 5). A ClustalW2 alignment of the two proteins also showed homology of the nucleotide-binding domain (NBD) of NLRP1 (FIG. 5) and full-length NLRP1 (FIG. 6) with KSHV Orf63. However, KSHV Orf63 does not contain the effector caspase activation and recruitment domain (CARD) or pyrin domain (PYD) of NLRP1, which are required for its activation, indicating that Orf63 may function as an inhibitor of NLRP1.

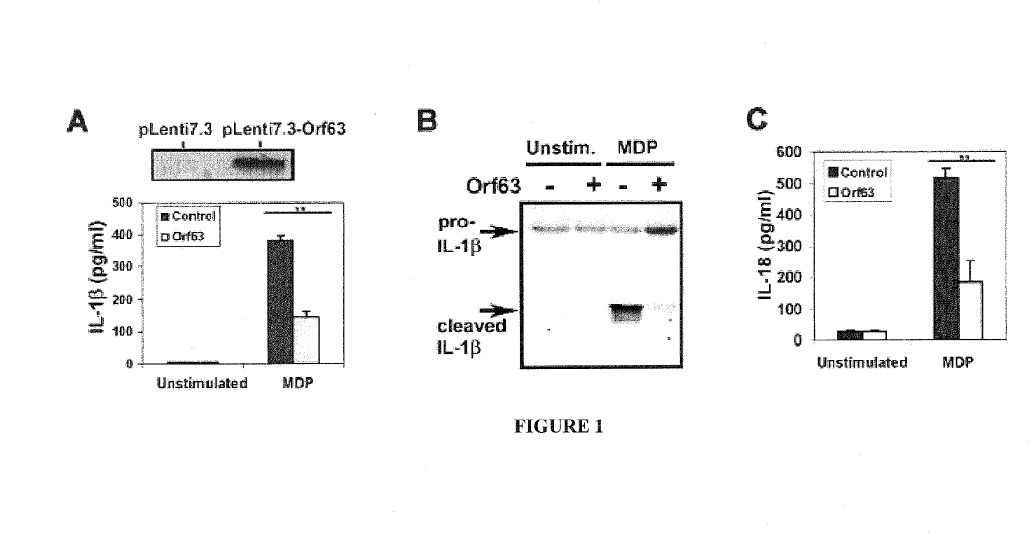

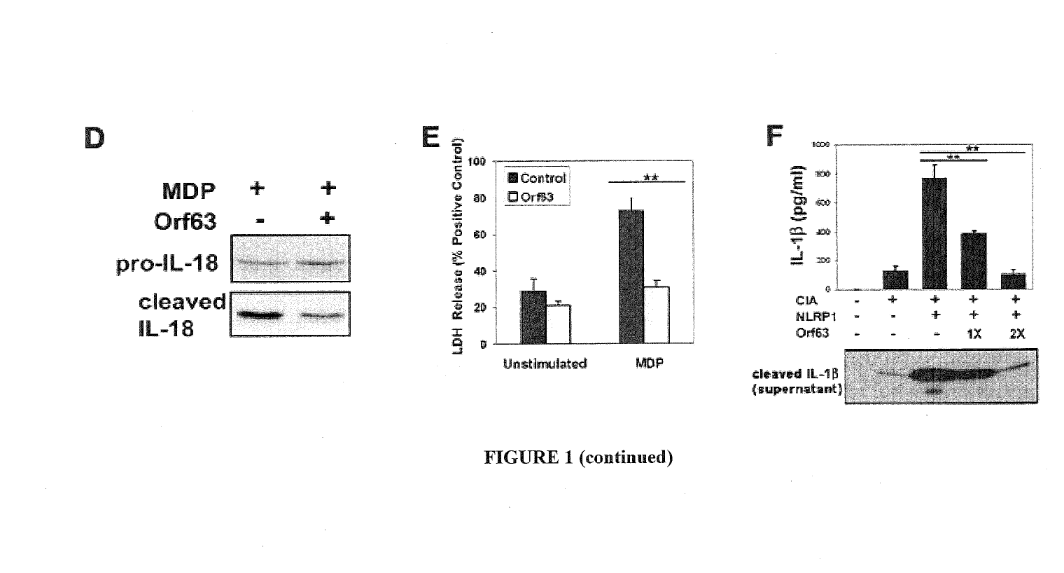

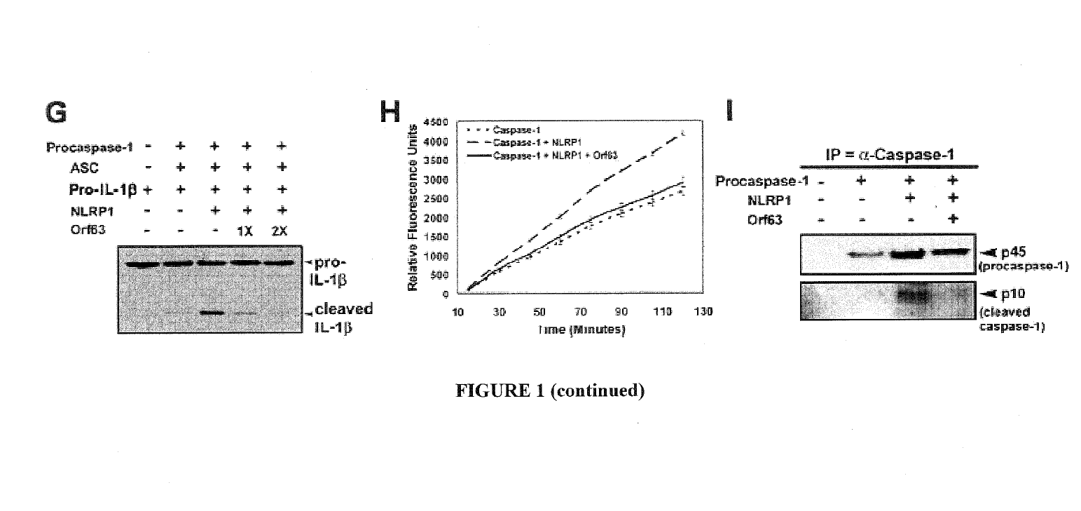

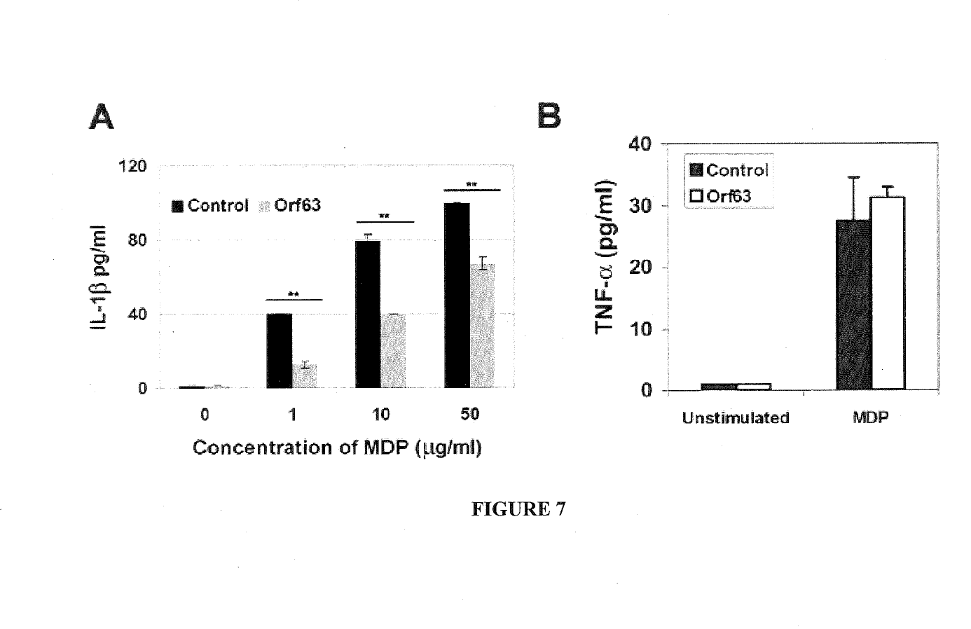

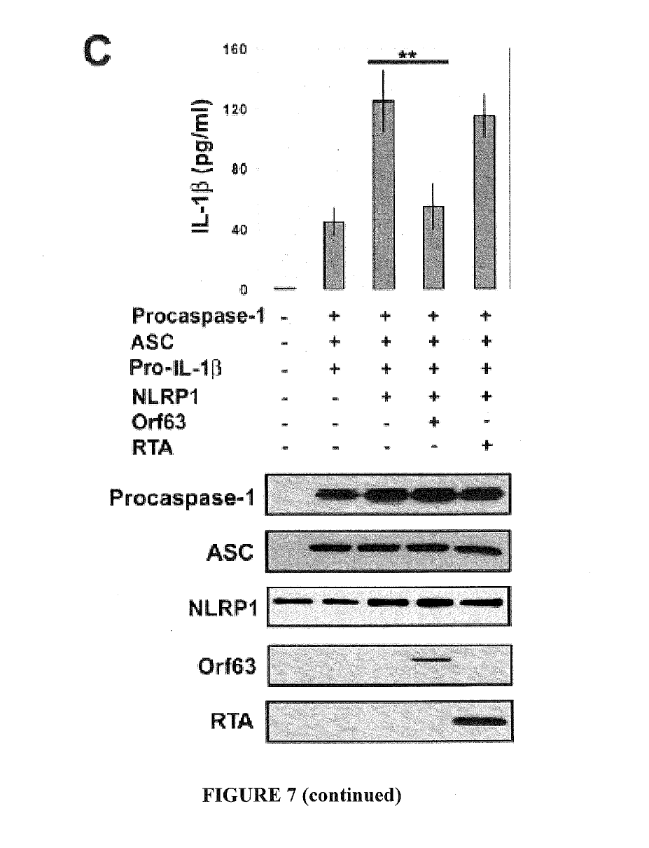

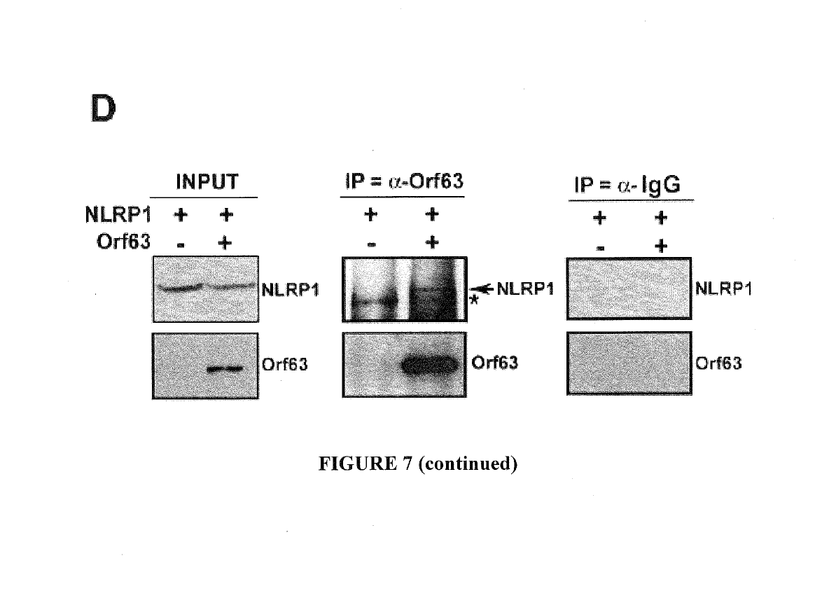

To investigate whether Orf63 inhibits NLRP1 activity, THP-1 cells stably expressing Orf63 and control cells were primed with lipopolysaccharide (LPS) to upregulate IL-1β transcription, followed by stimulation of NLRP1 with the bacteria cell wall constituent, muramyldipeptide (MDP). We found that Orf63 expression significantly inhibited MDP-induced IL-1β and IL-18 production compared to control cells (FIG. 1A-D and FIG. 7A). Furthermore no changes in the inflammasome-independent cytokine, tumor necrosis factor (TNF)-α, were observed, confirming the specificity of Orf63 inhibition of the NLRP1 inflammasome (FIG. 7B).

Inflammasome activation leads to production of proinflammatory cytokines and eventual cell death. In cells expressing Orf63, NLRP1-dependent cell death (as measured by lactate dehydrogenase (LDH) activity) was significantly inhibited compared to vector alone (FIG. 1E), demonstrating that Orf63 protects cells from NLRP 1-dependent cell death.

To further examine the role of Orf63 in blocking NLRP1, we transfected 293T cells with Orf63 and the NLRP1 inflammasome components ASC, procaspase-1 and pro-IL-1β (14). Reconstitution of the NLRP1 inflammasome resulted in an increase in IL-1β secretion, which was inhibited by expression of Orf63 in a dose-dependent fashion (FIGS. 1F and G). In contrast, another KSHV viral protein, replication and transcription activator (RTA) was unable to inhibit IL-1β secretion (FIG. 7C). We also investigated the ability of Orf63 to inhibit caspase-1 enzymatic activity. Transfection of procaspase-1 and NLRP1 into 293T cells resulted in increased caspase-1 specific activity that was inhibited by co-expression of Orf63 (FIG. 1H). Furthermore, NLRP1-induced proteolytic processing of procaspase-1 to activated caspase-1 was also inhibited (FIG. 1I).

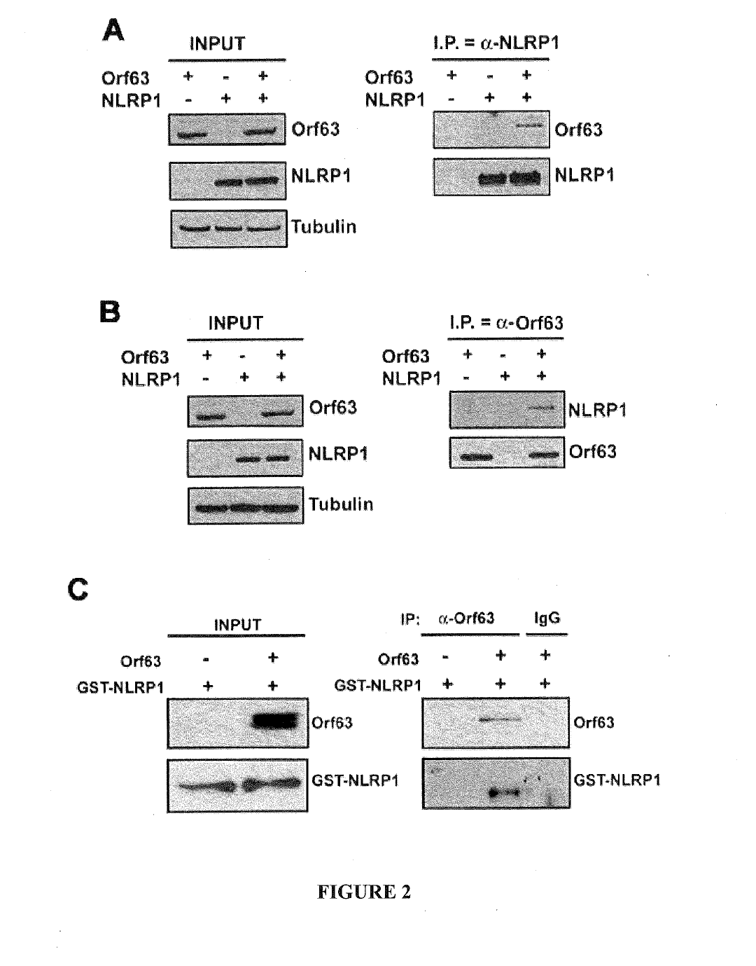

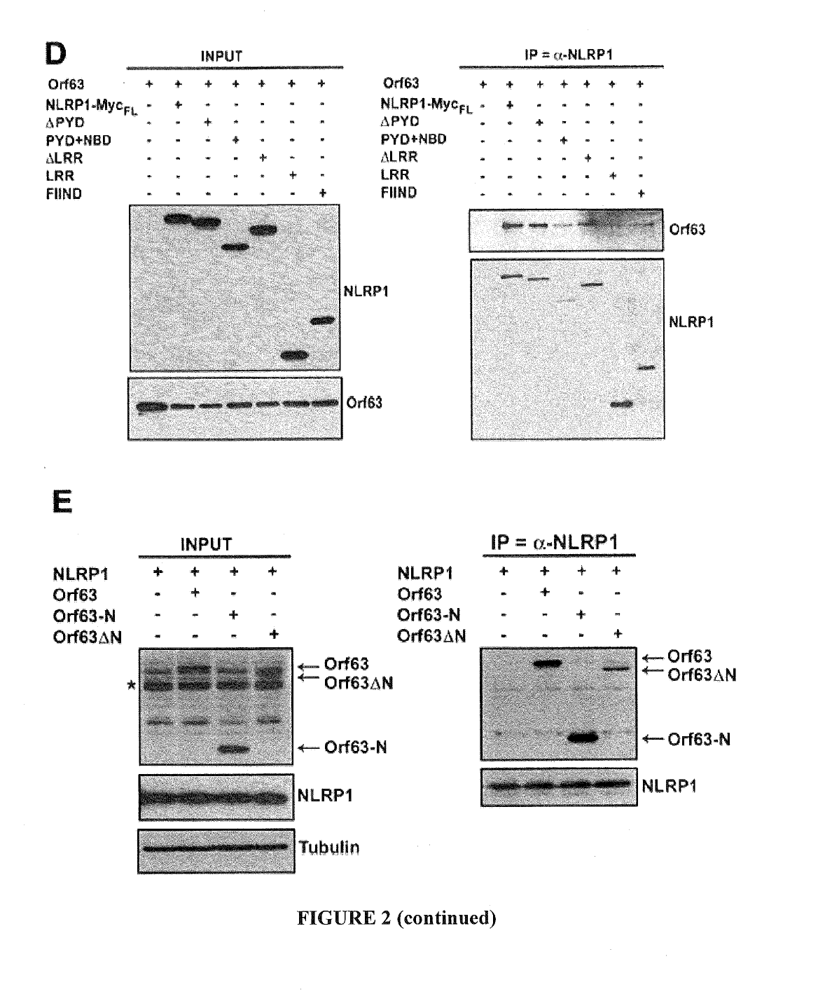

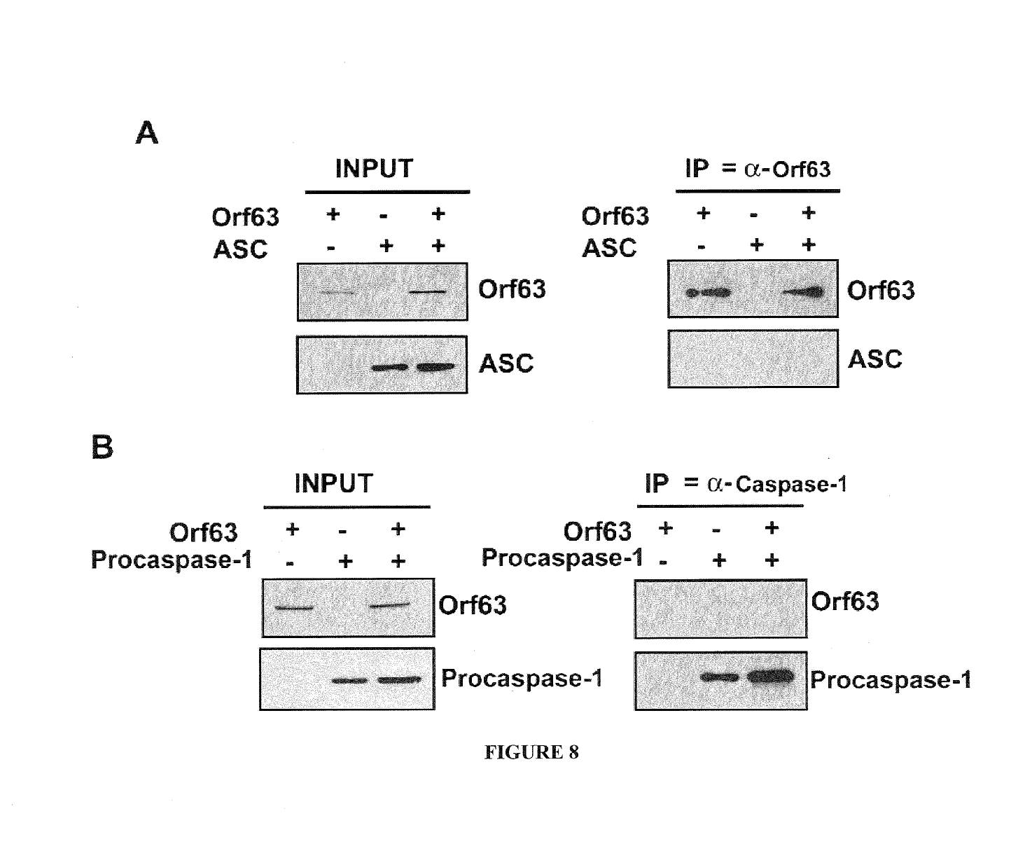

Next, we investigated whether Orf63 could interact with NLRP1. Orf63 co-immunoprecipitated with NLRP1, and vice versa, in 293T cells co-transfected with plasmids to express these proteins (FIGS. 2A and B). Endogenous NLRP1 was also shown to interact with Orf63 when Orf63 was expressed in THP-1 cells (FIG. 7D). Thus, Orf63 is capable of interacting with NLRP1 and/or in a complex with components of the NLRP1 inflammasome. To investigate the latter scenario, we tested the ability of Orf63 to interact with ASC or caspase-1. We found that Orf63 did not interact with either ASC or caspase-1 in the absence of NLRP1 (FIG. 8). Next, Orf63 was immunoprecipitated from a mixture of purified Orf63 made in bacteria and purified NLRP1 made in insect cells, and we found that NLRP1 protein co-immunoprecipitated with Orf63, suggesting that Orf63 directly interacts with NLRP 1 in the absence of other proteins (FIG. 2C).

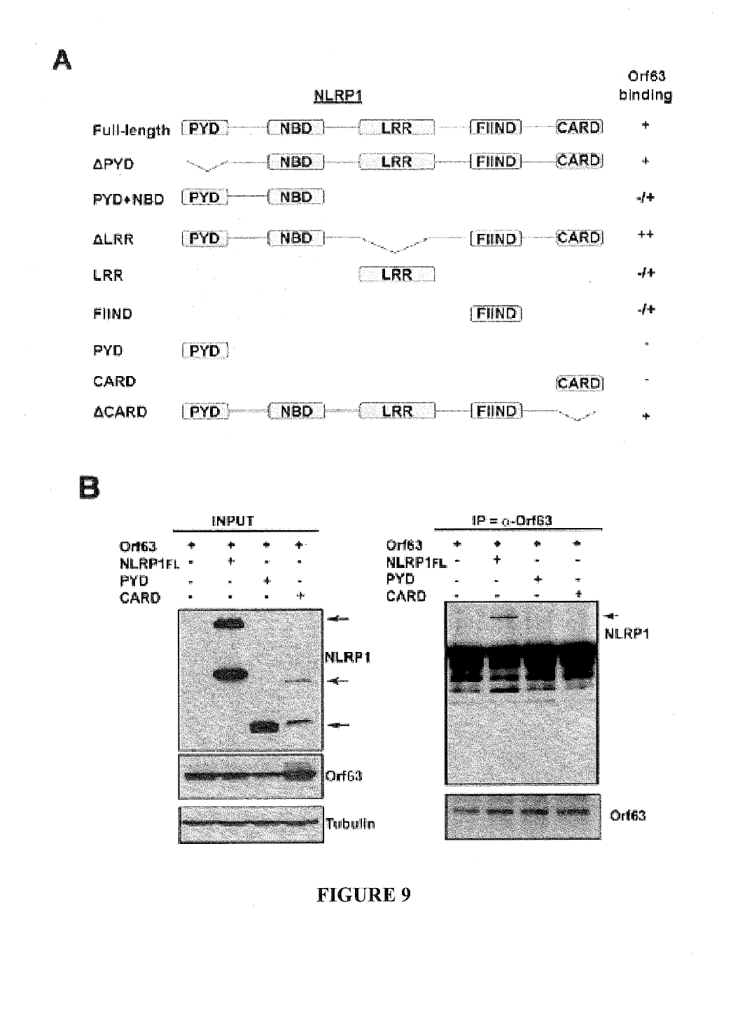

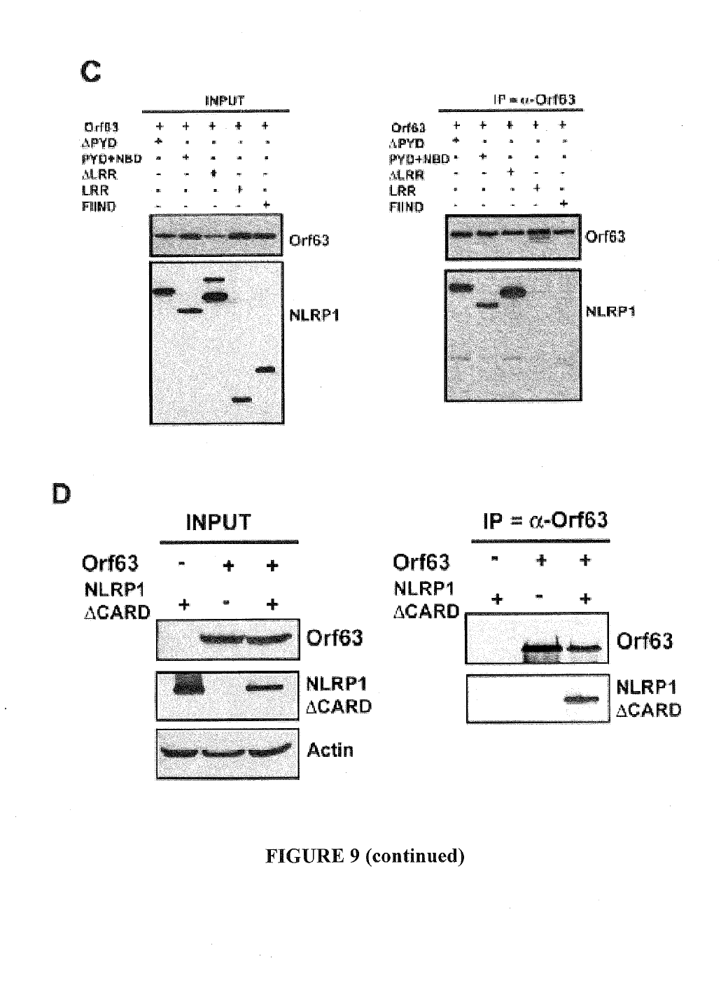

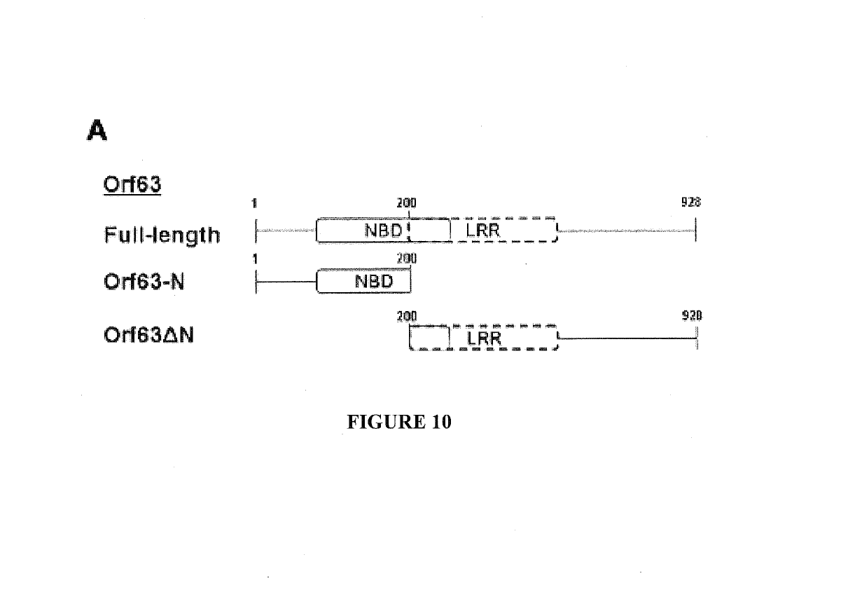

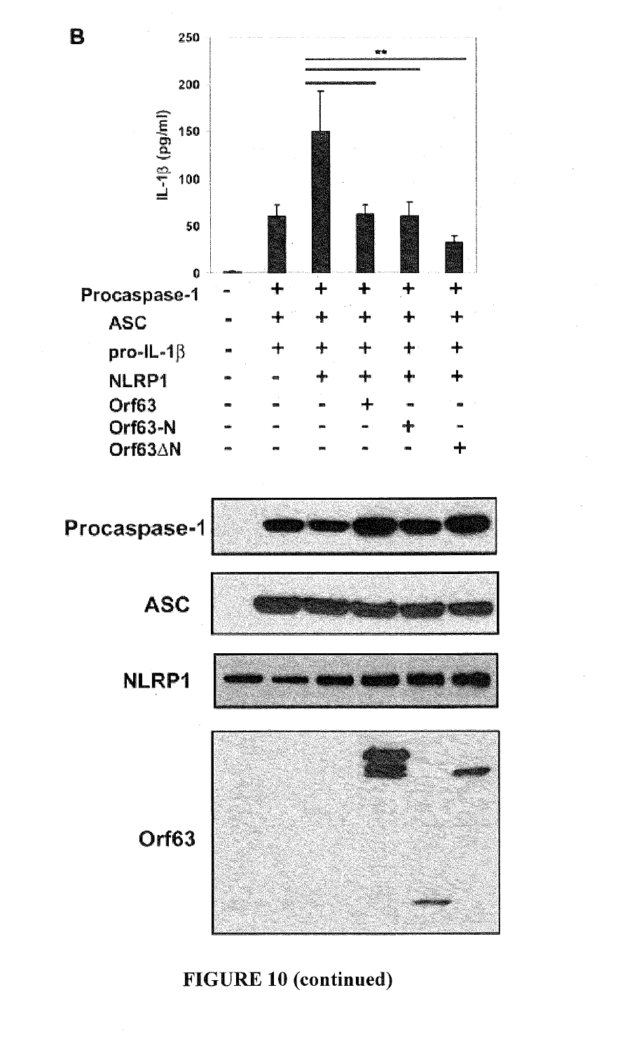

To identify the structural elements of NLRP1 that were required for interaction with Orf63, we tested several domain mutants of NLRP1 (15). KSHV Orf63 interacted with the NBD, LRR and FIIND (function to find) domains of NLRP1, whereas no interaction was observed with the PYD and CARD effector domains of NLRP1 (FIG. 2D and FIG. 9A-D). The LRR domain of NLRP1 is thought to negatively regulate its activation by folding back onto the NBD domains (14, 16). Interaction of Orf63 with NLRP1 suggests that Orf63 may be functioning similarly to the NLRP1 LRR to block activity. Sequence alignments indicated that Orf63 most closely aligned with the LRR and NBD domains of NLRP1 (FIG. 5A). Hence, we created mutants of Orf63; Orf63-N, which contains most of the region similar to NBD but lacks the region that aligns with the LRR, and Orf63ΔN, which contains the entire LRR and only a part of the NBD (FIG. 10A), and tested the ability of these mutants to bind NLRP1 by co-immunoprecipitation assays. Either domain was sufficient for interacting with NLRP1 (FIG. 2E) similar to previous reports on NLR proteins (17, 18). Furthermore, in NLRP1 inflammasome reconstitution assays, both Orf63 mutants were capable of inhibiting NLRP1 activity (FIG. 10B).

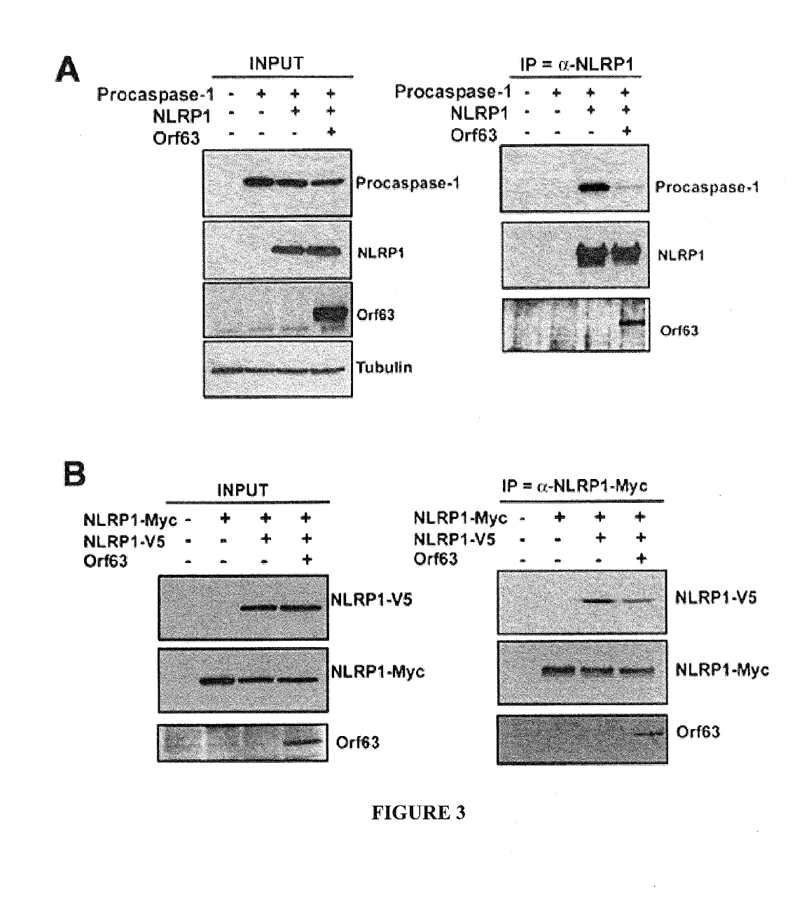

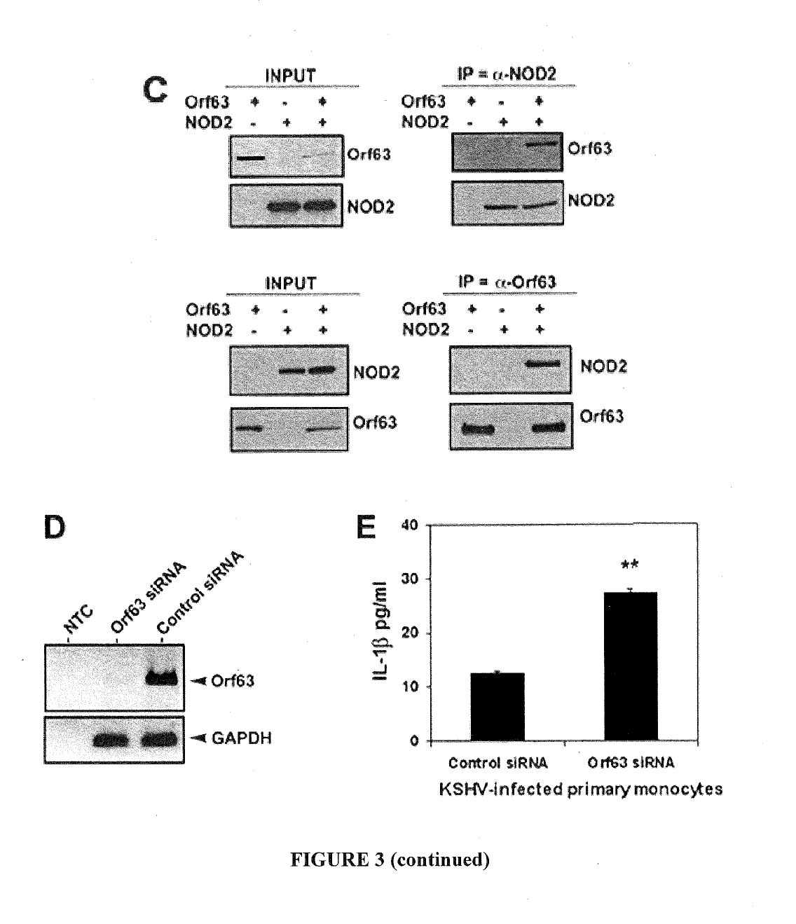

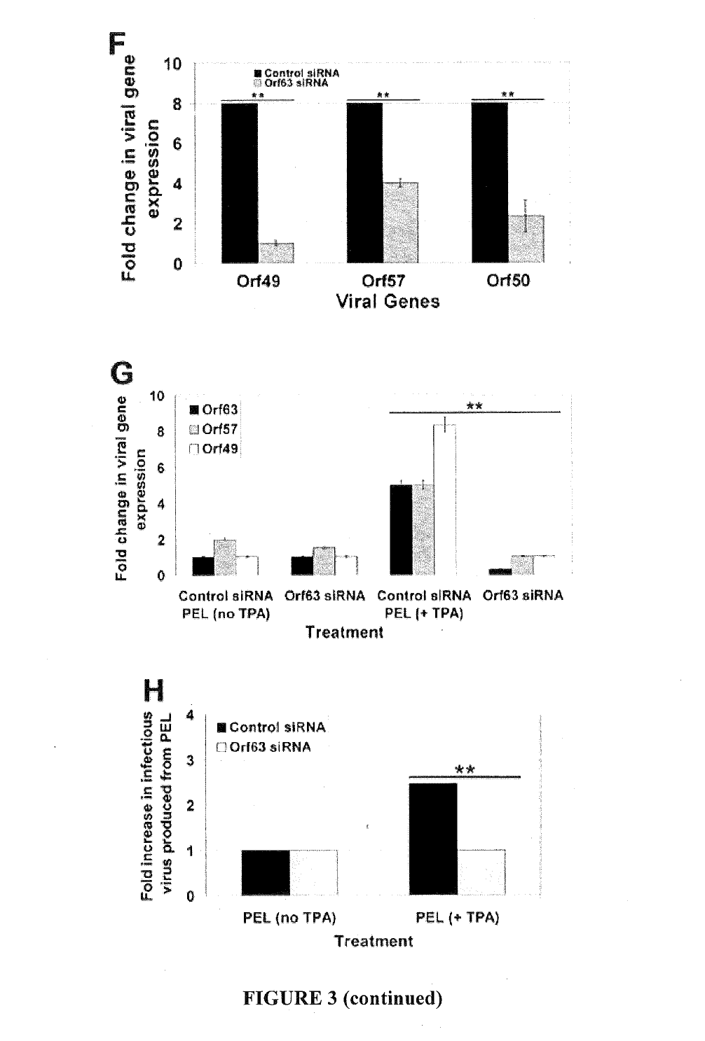

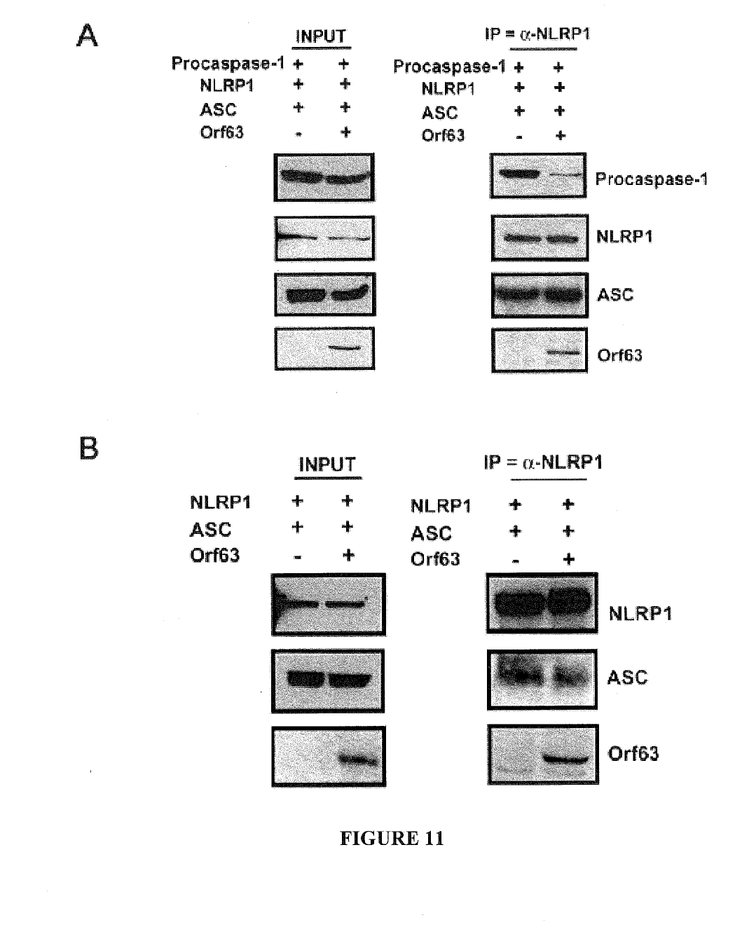

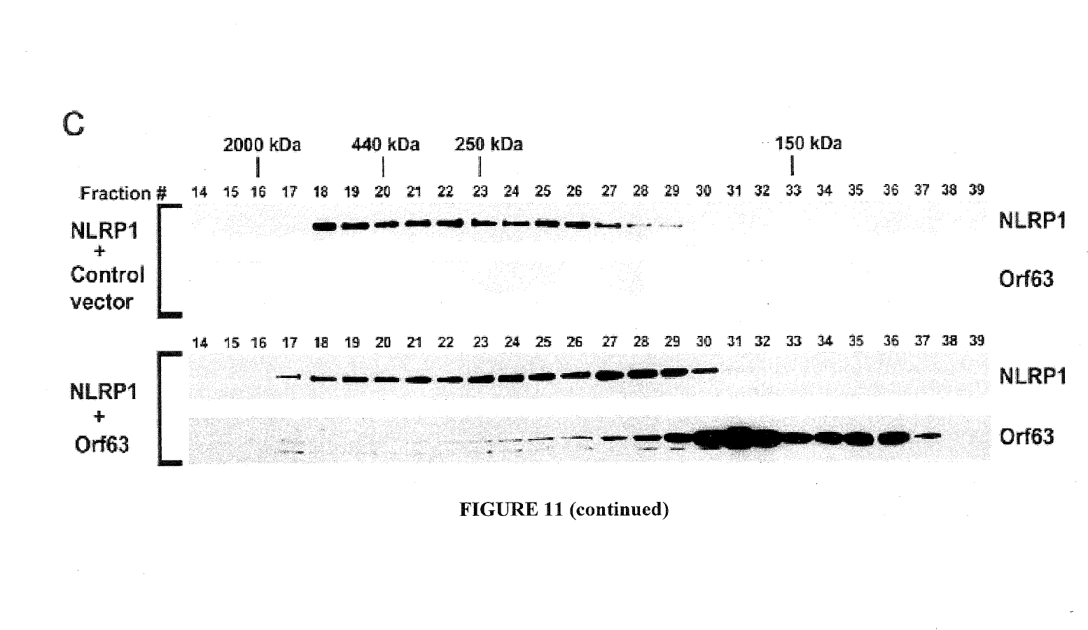

The NLRP1 inflammasome is comprised of multiple proteins complexed with NLRP1, caspase-1 and ASC (13). Our data demonstrate that Orf63 can inhibit NLRP1 function and can also interact with NLRP1. To determine the mechanism of NLRP1 inhibition, we tested the ability of Orf63 to bind NLRP1 and to inhibit the formation of the NLRP1 inflammasome. The presence of Orf63 inhibited the interaction of NLRP1 with procaspase-1, but not its interaction with ASC (FIG. 3A and FIGS. 11A and B). NLRP1 oligomerization has been shown to be required for activation (14). We confirmed that NLRP 1 self-associates and found that Orf63 blocked this self-association (FIG. 3B). Gel fractionation analysis under native conditions revealed that the presence of Orf63 caused more NLRP1 to fractionate in the lower molecular weight fractions, where Orf63 is also expressed (FIG. 11C). This provides further support that Orf63 inhibits NLRP1 oligomerization. Taken together, our findings indicate that Orf63 hinders inflammasome formation by both preventing NLRP1 oligomerization and inhibiting the association of NLRP1 with procaspase-1.

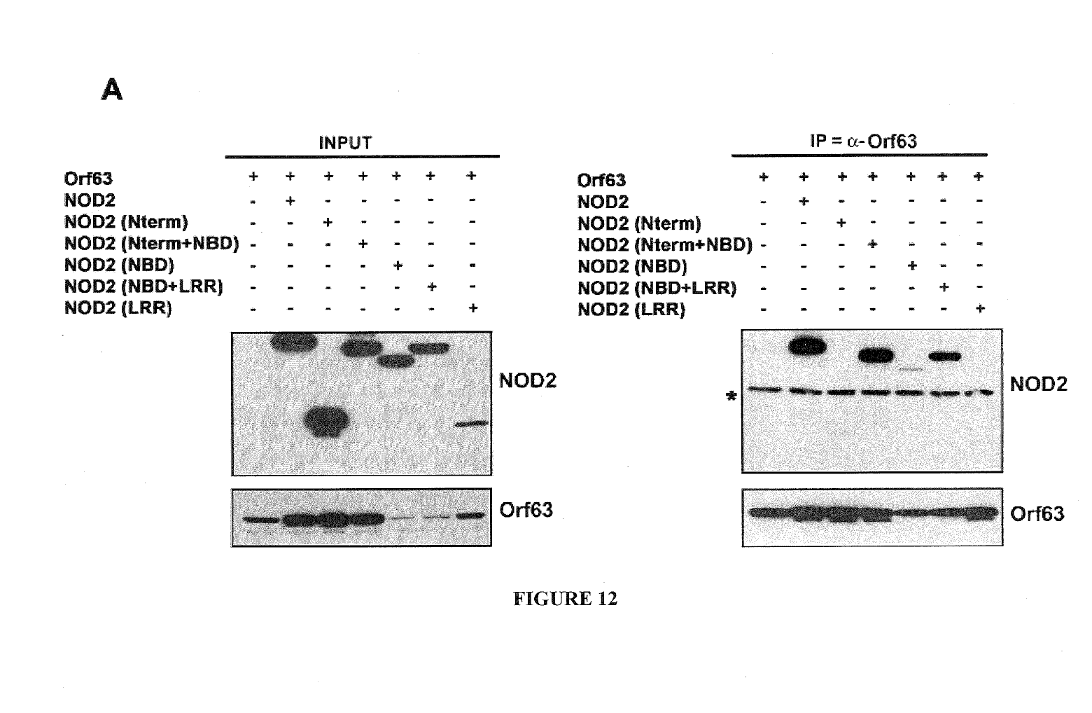

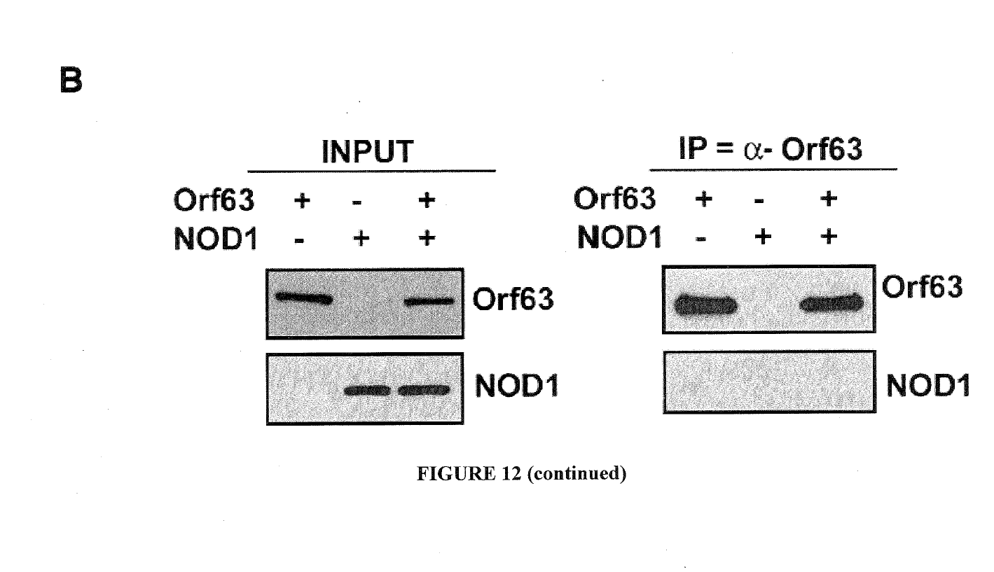

NLRP1 can also interact with NOD2 and this interaction enhances its activity in terms of inflammasome function (19). Reciprocal immunoprecipitations revealed that Orf63 interacts with NOD2 (FIG. 3C). Similar to NLRP1, the interaction of Orf63 with NOD2 required the NBD domain (FIG. 12A). In contrast, Orf63 did not interact with NOD1 (FIG. 12B).

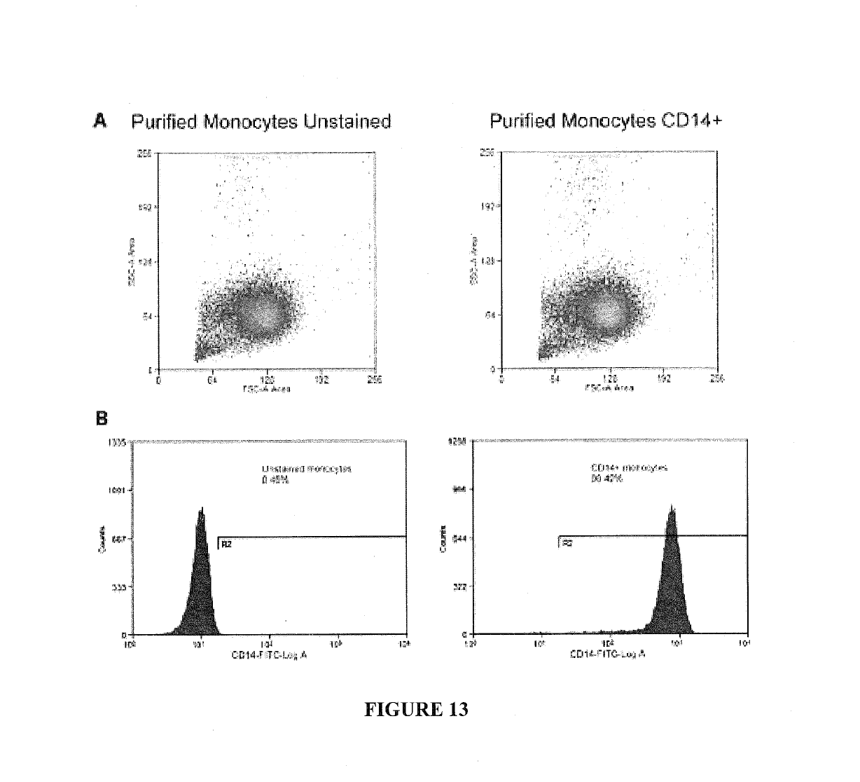

NLRP1 activation and subsequent IL-1β and IL-18 secretion is detrimental to viral infection. We hypothesized that inhibition of Orf63 would result in increased proinflammatory cytokine production during KSHV infection. KSHV has been shown to infect human monocytes (7), therefore we isolated primary human monocytes from blood from healthy donors (FIGS. 13A and B) and infected them with KSHV in the presence and absence of siRNAs against Orf63 (FIG. 3D). We detected increased IL-1β expression and decreased viral gene expression in KSHV-infected monocytes transfected with siRNAs targeting Orf63 compared to non-targeting control (FIGS. 3E and F, respectively).

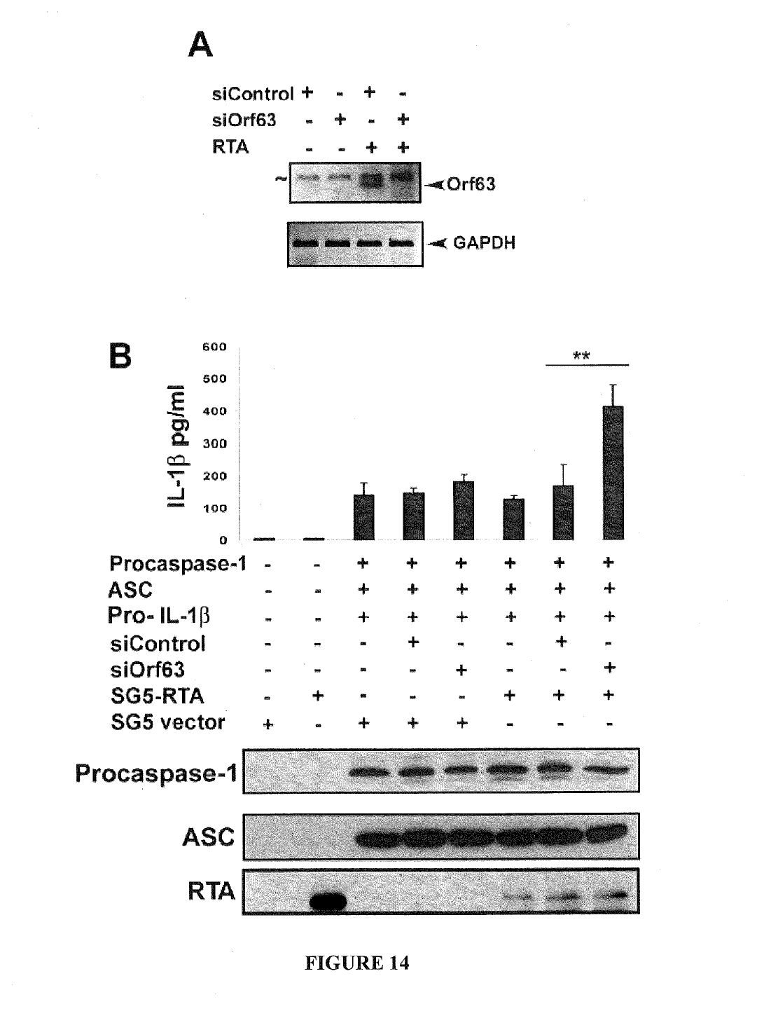

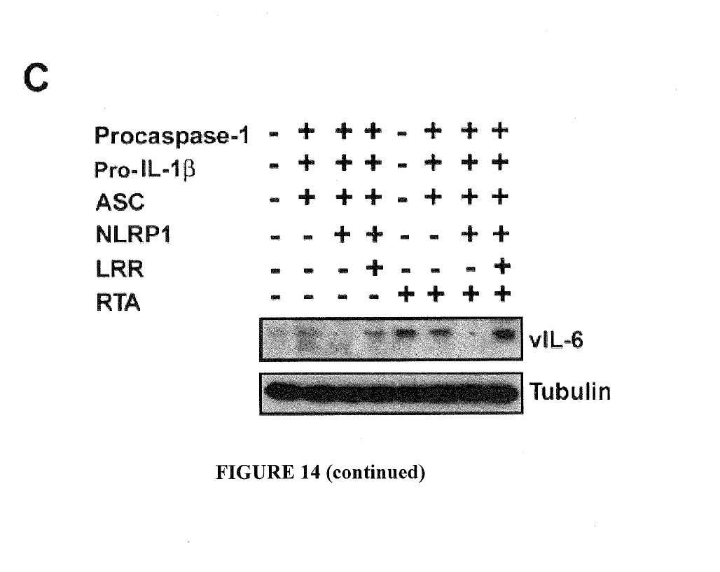

We next determined the effect of Orf63 during viral reactivation. KSHV-293T cells containing endogenous NLRP1 were transfected with a plasmid encoding RTA to induce KSHV reactivation and lytic gene expression (20). Knockdown of Orf63 resulted in a statistically significant increase in IL-1β expression compared to non-targeting control (FIGS. 14A and B). Reconstitution of cells with the NLRP1 inflammasome inhibited expression of KSHV reactivation as measured by expression of the vIL-6 lytic protein (FIGS. 14C); however, when only the LRR domain of NLRP1, which is an inhibitor of NLR activity (14, 16, 21), was co-transfected with the inflammasome components, vIL-6 expression was restored (FIG. 14C).

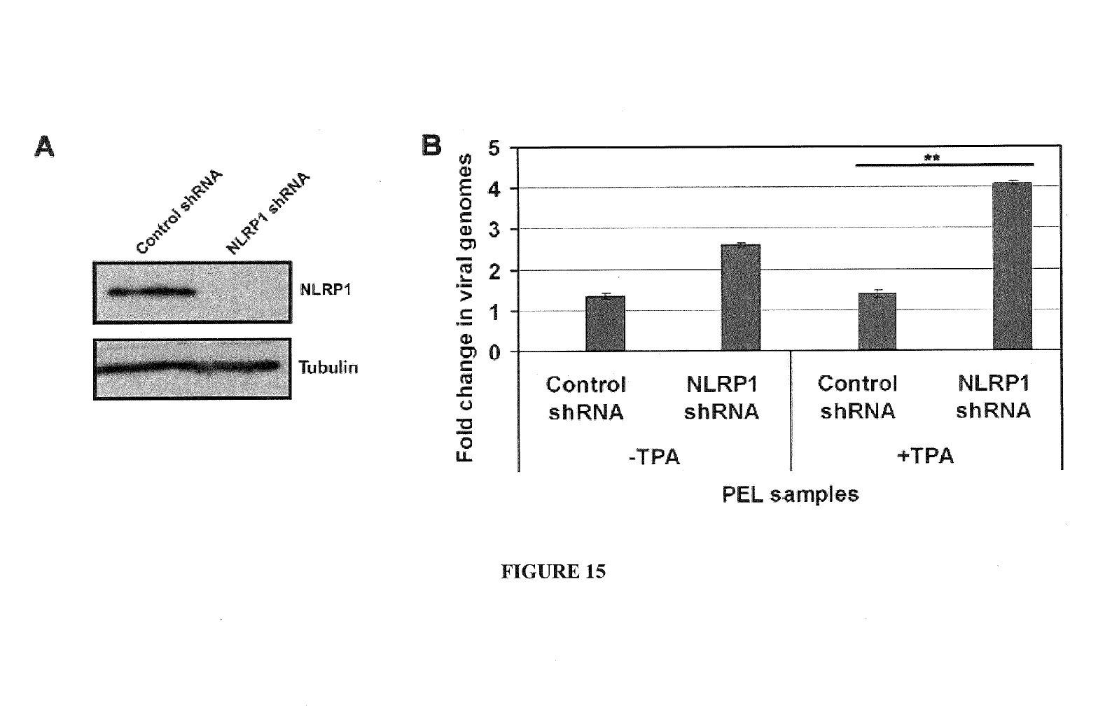

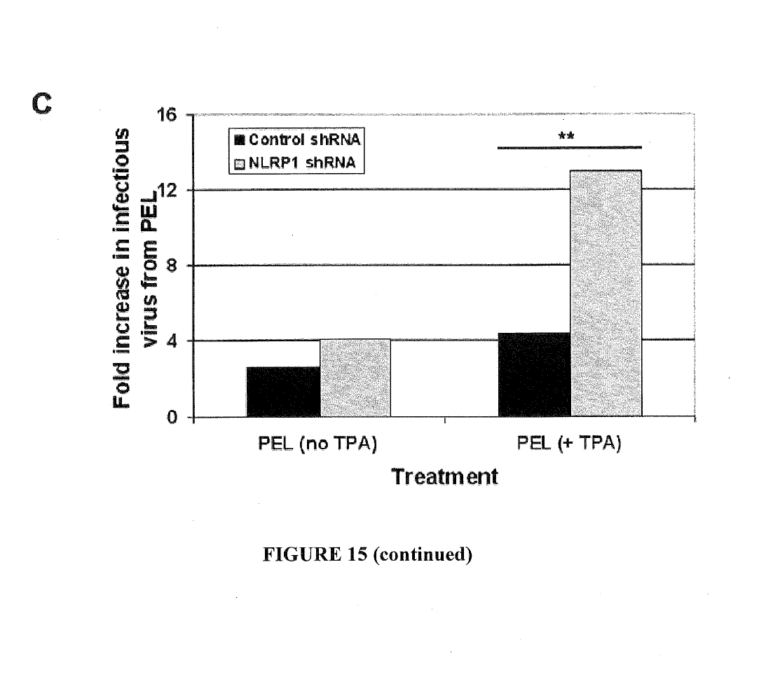

Similarly, when NLRP1 expression was knocked-down in KSHV-infected latent BCBL-1 primary effusion lymphoma (PEL) cells (FIG. 15A), we detected an increase in viral genomes and infectious virus, which was enhanced after induction of lytic replication (FIGS. 15B and C). To confirm that Orf63 is necessary for KSHV lytic gene expression, we inhibited Orf63 expression in BCBL-1 PEL cells using Orf63 siRNA. Orf63 siRNA transfected PEL showed a significant loss of Orf63 expression (FIG. 3G). Further, Orf63 knockdown resulted in the loss of lytic gene expression as measured by loss of Orf49 and Orf57 viral lytic transcript expression (FIG. 3G). After knockdown of Orf63 in reactivated PEL, supernatant was transferred to naïve Vero cells and viral infectivity on Vero cells was quantitated by real-time PCR. We found that Orf63 knockdown resulted in a block of infectious virus produced during PEL reactivation (FIG. 3H). Taken together, our data show that Orf63 can block NLRP1 activation and the production of IL-1β. This function of Orf63 appears to be critical for viral gene expression and viral genome replication during KSHV primary infection as well as KSHV reactivation from latency.

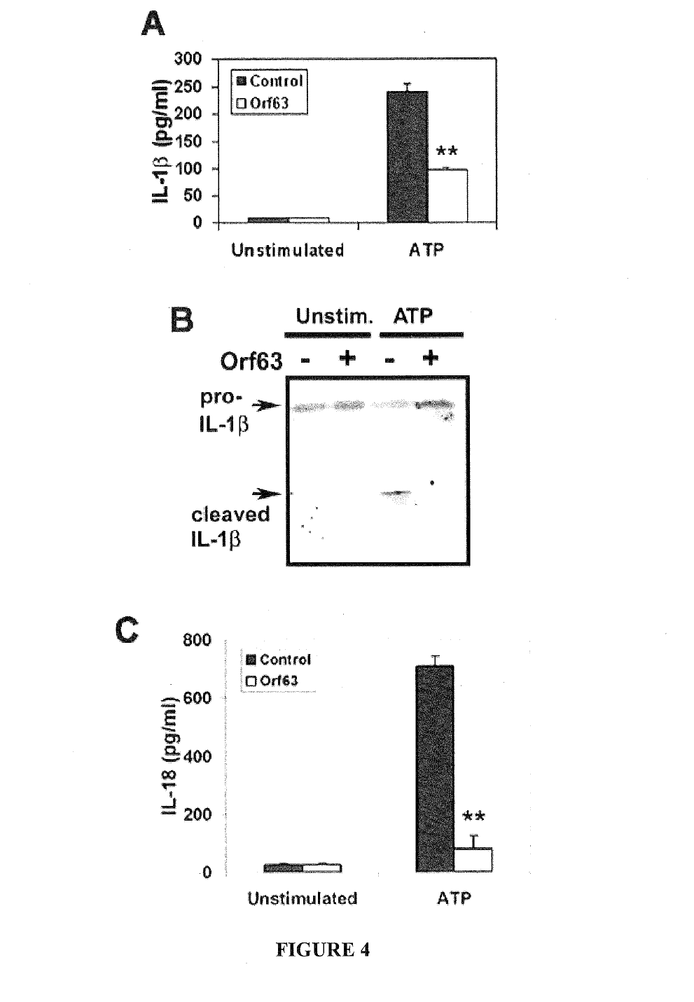

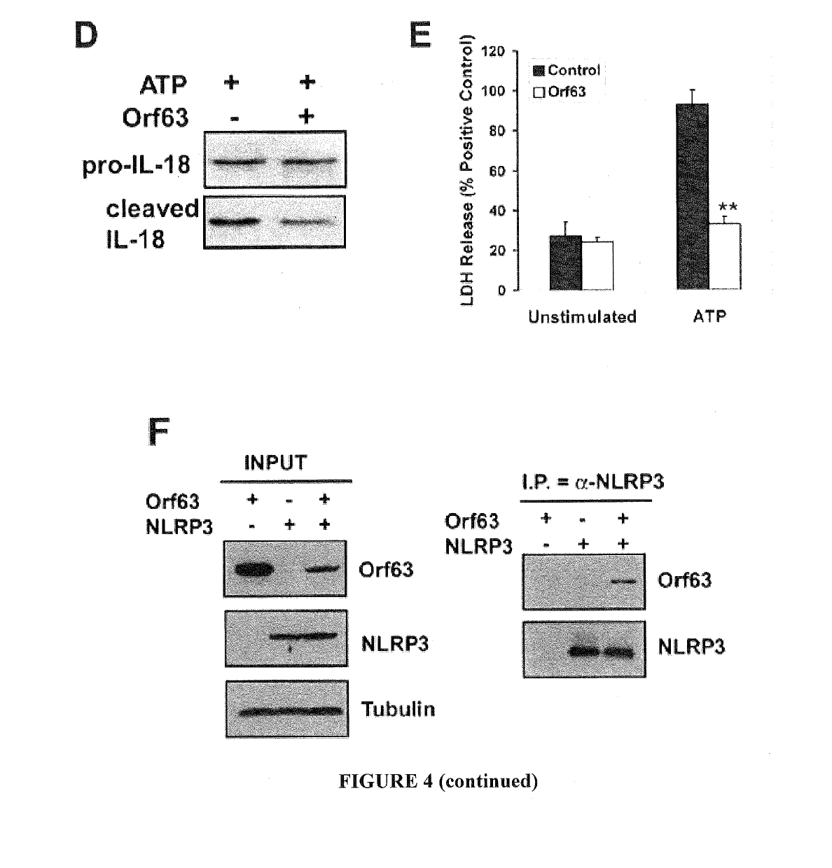

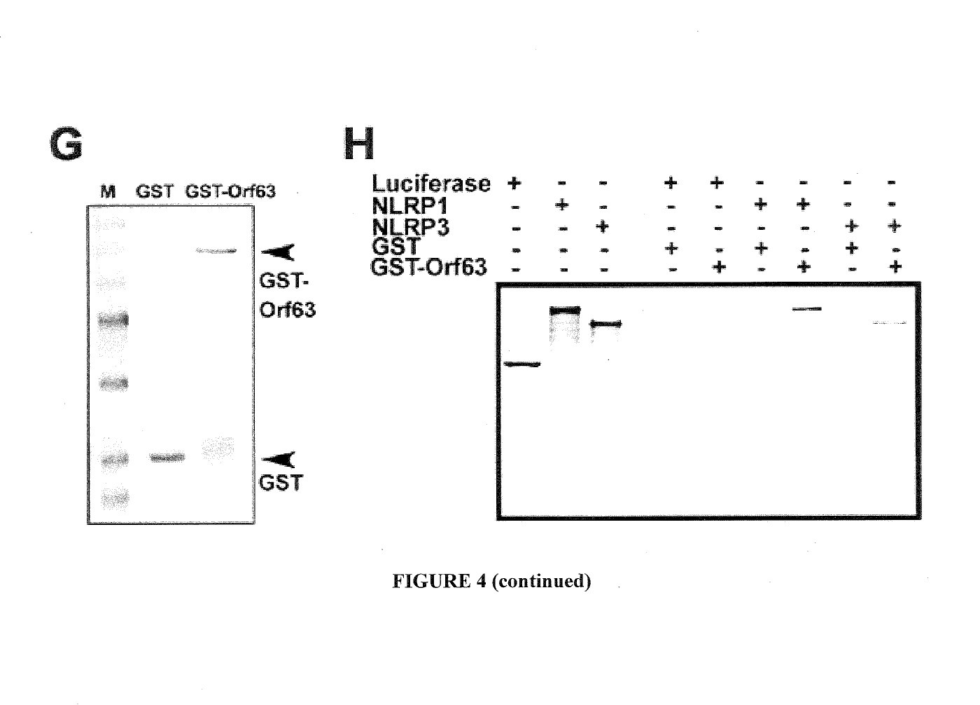

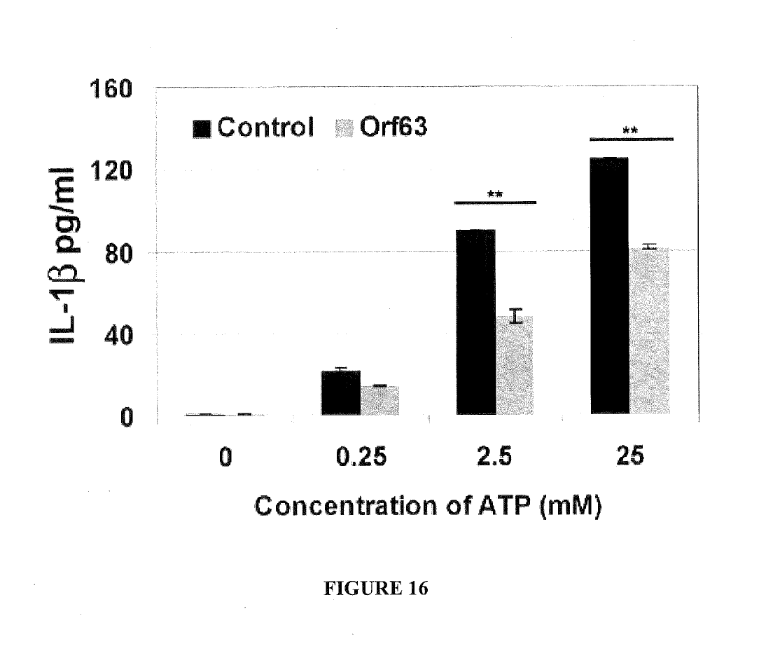

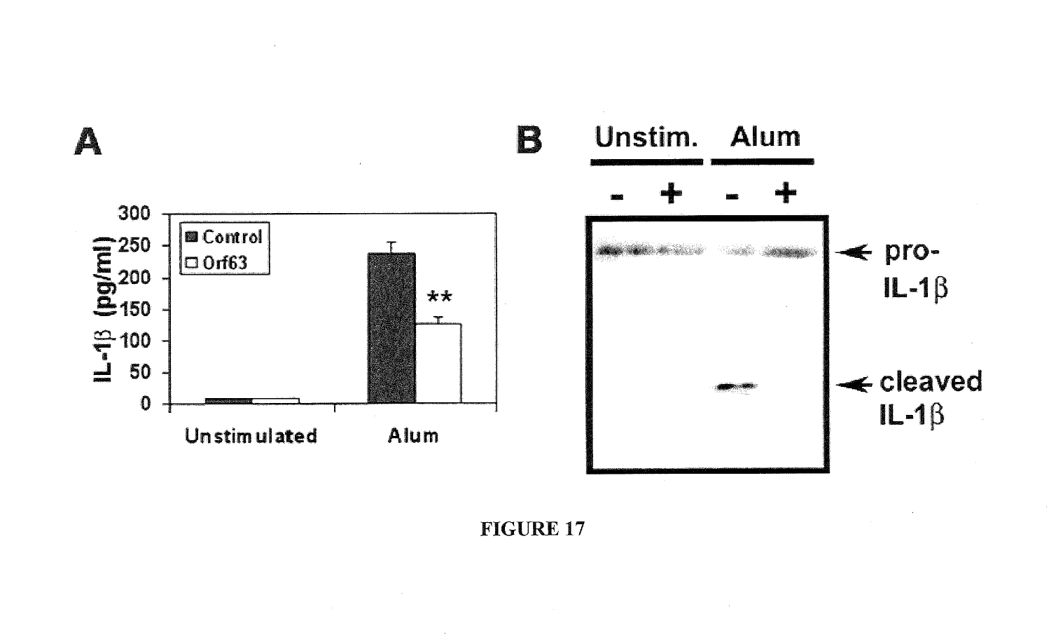

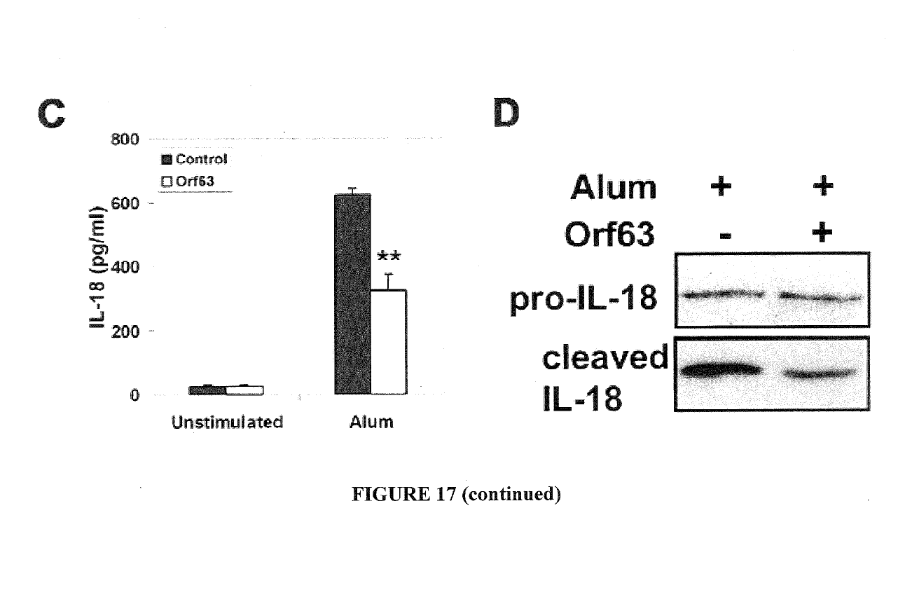

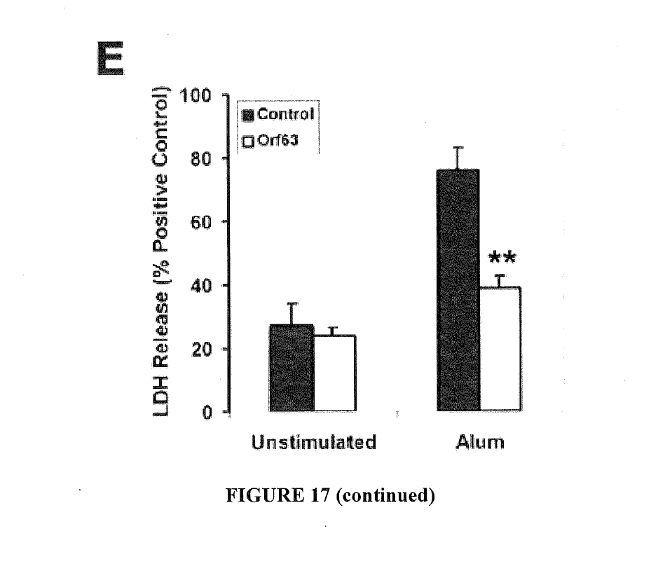

BLASTP alignments revealed homology of Orf63 to the NBD and LRR domains of NLRP1, which are conserved motifs among the NLR family members. To investigate whether Orf63 could also inhibit the activation of other NLR family members, we stimulated THP-1-Orf63 or control THP-1 cells with several NLRP3 agonists (12). Orf63 inhibited IL-1β and IL-18 cleavage and production after treatment with the NLRP3 agonists, ATP and Alum (FIG. 4A-D, FIG. 16 and FIGS. 17A-D). Orf63 also protected cells from NLRP3-dependent cell death in response to these agonists as measured by a LDH release assay (FIG. 4E and FIG. 17E). Orf63 co-immunoprecipitated with NLRP3 when both proteins were exogenously expressed in 293T cells (FIG. 4F). Furthermore, ³⁵S-methionine-labeled NLRP1 and NLRP3 bound to purified GST-Orf63 protein (FIGS. 4G and H).

KSHV exists in diverse cell types including monocytes, B cells, epithelial, and endothelial cells (22-24). NLRP1 is ubiquitously expressed compared to NLRP3, which is more restricted in its expression (25, 26). Herpesviruses establish life-long latency and must encode proteins that function in the context of all types of cellular events including adverse circumstances.

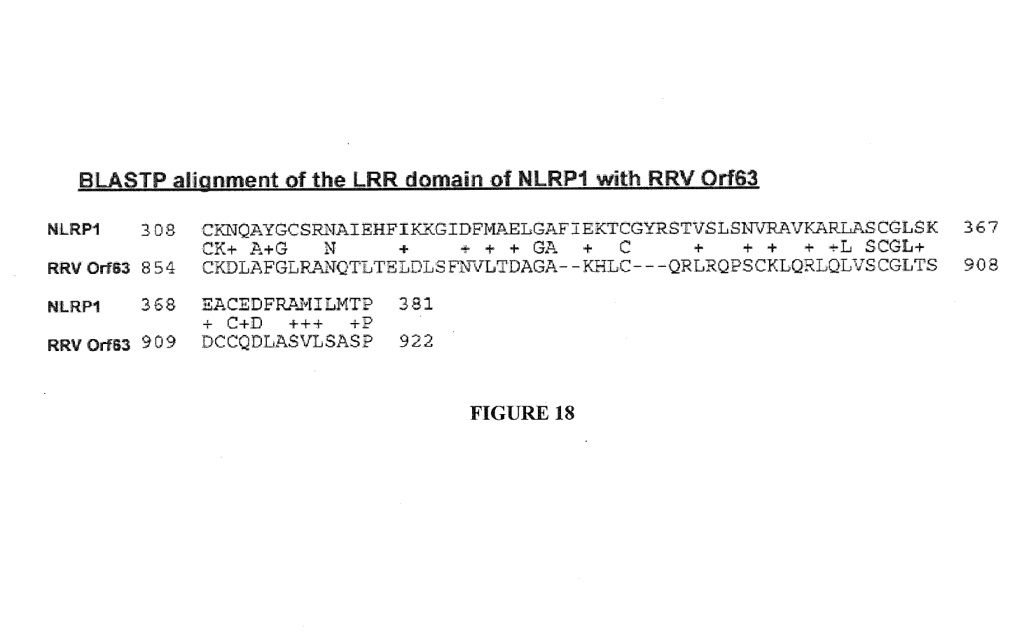

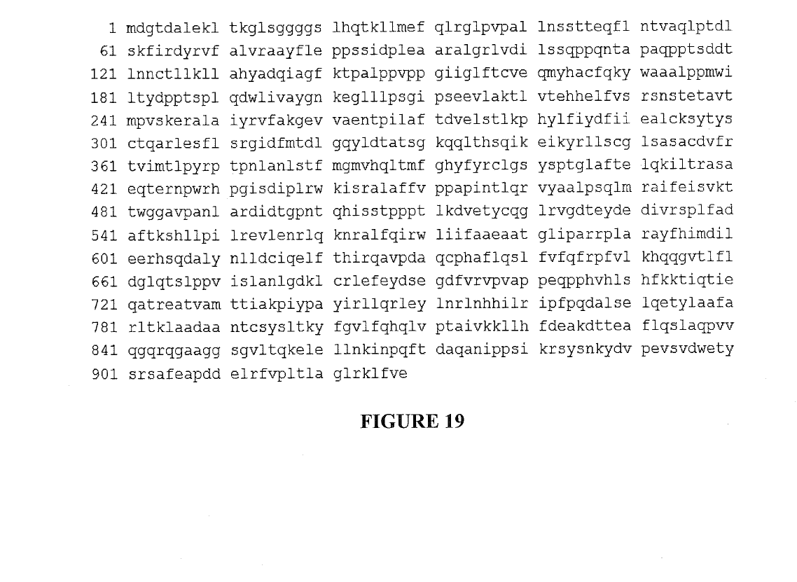

The NLR nomenclature requires that in order to be classified as a NLR, the protein must contain a NBD and a LRR domain, which are two domains that are evolutionarily conserved in all NLRs (27). Poxviruses encode a pyrin-only containing protein named M13L that inhibits IL-1β and IL-18 secretion (28) through a different mechanism since M13L lacks both a NBD and a LRR domain. KSHV Orf63 does show homology to both the NBD and LRR domains of NLRs and is a homolog of NLRP1. We also found that another herpesvirus encodes a viral homolog of NLRP1 (FIG. 18). This suggests that the targeting of NLR proteins might play a very important role in the herpesvirus lifecycle.

KSHV encodes a viral homolog of cellular NLRP1 without the CARD and PYD effector domains of its cellular counterpart. Although Orf63 did not demonstrate significant similarity to NLRP3, it blocked NLRP3 activity, suggesting that Orf63 is capable of broad inhibition of NLR inflammasome responses. Thus, during the course of evolution with its human host, KSHV has usurped and modified a cellular NLR gene to inhibit the host inflammasome response.

In summary, this is the first report of molecular piracy of a NLR protein by a virus. KSHV Orf63, the viral homolog of NLRP1, inhibits activation of the cellular NLRP1 inflammasome. These data indicate that KSHV has usurped and modified NLRP1 to encode a viral homolog containing a LRR domain without the CARD and PYD effector domains of its cellular counterpart. KSHV Orf63 inhibited NLRP1-dependent caspase-1 enzymatic activity and cytokine expression. KSHV Orf63 also inhibited NLRP3-dependent activity and protected cells from NLRP3 induced cell death. Although Orf63 did not demonstrate significant similarity to NLRP3 by BLASTP alignment, it blocked NLRP3 activity, indicating that Orf63 is capable of broad inhibition of NLR inflammasome responses. These observations also suggest that several NLRs may contribute to control of viral infection and that multiple pathways may therefore be targeted by viral proteins to overcome host cell defenses. Thus, these findings suggest that during the course of evolution with its human host, KSHV has pirated a cellular NLR gene to inhibit the host inflammasome response.

In vivo studies of Orf63 and peptides and fragments thereof: The nucleotide-binding leucine-rich repeat (NLR) family of innate immune receptors is associated with various autoinflammatory, autoimmune and metabolic disorders as well as cancer development (33). In the present invention, we discovered that Orf63, a protein encoded by Kaposi's sarcoma-associated herpesvirus (KSHV), inhibited inflammatory signaling mediated by NLR family members NLRP1, NLRP3 and perhaps NOD2 (35). Orf63 is a viral mimic of cellular NLRP1, having pirated conserved amino acids of NLRP1 that enables broad inhibition of NLRP1, NLRP3, NOD2 and likely additional NLRs since the conserved region is in the domain shared by all NLR family members. The conserved amino acids represent a minimal motif that can be synthesized as a small peptide inhibitor (Orf63p) and tested according to well known protocols for therapeutic benefit in treating NLR-associated diseases and disorders.

NLR-associated diseases have been studied using a variety of in-vivo mouse models, which can be utilized in the present invention to investigate the therapeutic effects of Orf63p. For example, atherosclerosis, an inflammatory disorder of the arterial wall, is modeled using Apo-E-deficient mice that are fed a high cholesterol diet (34). The intraperitoneal injection of cholesterol crystals induces acute inflammation that is dependent on NLRP3 activation, and links the pathogenesis of the disease to inflammasome signaling. Moreover, mouse models of type II diabetes that rely on the production of islet amyloid polypeptide and subsequent NLRP3 activation are well established and could be used to test the potential of Orf63p to inhibit inflammasome-dependent inflammation (36). Peptide administration has been successfully used to ameliorate inflammatory signaling by injection through a number of routes, including intraperitoneal, intradermal and intranasal injections (30-32). Thus, Orf63 ps of this invention will be tested in vivo using NLR-associated disorder mouse models by peptide injection through multiple routes to assess clinical efficacy. Since models such as the atherosclerosis and diabetes model and others are well established and have defined disease manifestation, this enables the use of protocols to evaluate Orf63 ps based on previously established peptide testing methods in vivo.

Peptide analysis: Studies will be conducted to test peptides that are 60 amino acids long located in the Orf63 NBD and LRR domains that span the 230-360 amino acid sequence of Orf63 (e.g., peptides having the amino acid sequence as shown in any of SEQ ID NOs:45-71). These peptides will be tested for the ability to block NLR-mediated IL1-beta upregulation and IL-18 upregulation in cell-based assays as described herein.

The above examples clearly illustrate the advantages of the invention. Although the present invention has been described with reference to specific details of certain embodiments thereof, it is not intended that such details should be regarded as limitations upon the scope of the invention except as and to the extent that they are included in the accompanying claims.

Throughout this application, various patents, patent publications and non-patent publications are referenced. The disclosures of these patents, patent publications and non-patent publications in their entireties are incorporated by reference herein into this application in order to more fully describe the state of the art to which this invention pertains.

### REFERENCES

1. E. Cesarman, Y. Chang, P. S. Moore, J. W. Said, D. M. Knowles, N Engl J Med. 332, 1186 (1995).
2. Y. Chang et al., Science. 266, 1865 (1994).
3. L. Coscoy, Nat Rev Immunol. 7, 391 (2007).
4. T. Kawai, S. Akira, Nat 11, 373 (2010).
5. E. Meylan, J. Tschopp, M. Karin, Nature. 442, 39 (2006).
6. S. M. Gregory et al., Proc Natl Acad Sci USA. 106, 11725 (2009).
7. J. West, B. Damania, J. Virol. 82, 5440 (2008).
8. C. Boschan et al., Am J Med Genet A. 140, 883 (2006).
9. Y. Jin et al., N Engl J Med 356, 1216 (2007).
10. T. Lequerre et al., Rheumatology (Oxford). 46, 709 (2007).
11. S. L. Masters, A. Simon, I. Aksentijevich, D. L. Kastner, Annu Rev Immunol. 27, 621 (2009).
12. K. Schroder, J. Tschopp, Cell. 140, 821 (2010).
13. F. Martinon, K. Burns, J. Tschopp, Mol. Cell. 10, 417 (2002).
14. B. Faustin et al., Mol. Cell. 25, 713 (2007).
15. J. M. Bruey et al., Cell. 129, 45 (2007).
16. T. Tanabe et al., Embo J 23, 1587 (2004).
17. J. S. Damiano, V. Oliveira, K. Welsh, J. C. Reed, Biochem J. 381, 213 (2004).
18. S. B. Hake et al., Mol Cell Biol. 20, 7716 (2000).
19. L. C. Hsu et al., Proc Nall Acad Sci USA. 105, 7803 (2008).
20. R. Sun et al., Proc Natl Acad Sci USA. 95, 10866 (1998).
21. J. L. Poyet et al., J Biol Chem 276, 28309 (Jul. 27, 2001).
22. C. Blasig et al., J Virol. 71, 7963 (1997).
23. P. Monini et al., Blood. 93, 4044 (1999).
24. J. Pauk et al., N Engl J Med. 343, 1369 (2000).
25. T. Hlaing et al., J Biol Chem. 276, 9230 (2001).
26. G. A. Manji et al., J Biol. Chem. 277, 11570 (2002).
27. J. P. Ting et al., Immunity. 28, 285 (2008).
28. J. B. Johnston et al., Immunity. 23, 587 (2005).
29. B. Damania, J. C. Alwin, Genes Dev 10, 1369 (Jun. 1, 1996).
30 Briggs et al. 1995. Peptides inhibit selectin-mediated cell adhesion in vitro, and neutrophil influx into inflammatory sites in vivo. Glycobiology 5:583-8.
31. Chima et al. 2011. C-peptide, a novel inhibitor of lung inflammation following hemorrhagic shock. Am J Physiol Lung Cell Mol Physiol 300:L730-9. Epub 2011 March 11.
32. Cooper et al. 2001. Attenuation of interleukin 8-induced nasal inflammation by an inhibitor peptide. Am J Respir Crit. Care Med. 163:1198-205.
33. Davis et al. 2011. The inflammasome NLRs in immunity, inflammation, and associated diseases. Annu Rev Immunol 29:707-35.
Duewell et al. 2010. NLRP3 inflammasomes are required for atherogenesis and activated by cholesterol crystals. Nature 464:1357-61.
35. Gregory et al. 2011. Discovery of a viral NLR homolog that inhibits the inflammasome. Science 331:330-4.
36. Masters et al. 2010. Activation of the NLRP3 inflammasome by islet amyloid polypeptide provides a mechanism for enhanced IL-1beta in type 2 diabetes. Nat Immunol 11:897-904. Epub 2010 Sep. 12.

TABLE 1
KSHV Orf63 peptides

```
    1. MDGTDALEKLTKGLSGGGGS (SEQ ID NO: 1)
    2. ALEKLTKGLSGGGGSLHQTK (SEQ ID NO: 2)
    3. TKGLSGGGGSLHQTKLLMEF (SEQ ID NO: 3)
    4. GGGGSLHQTKLLMEFQLRGL (SEQ ID NO: 4)
    5. LHQTKLLMEFQLRGLPVPAL (SEQ ID NO: 5)
    6. LLMEFQLRGLPVPALLNSST (SEQ ID NO: 6)
    7. QLRGLPVPALLNSSTTEQFL (SEQ ID NO: 7)
    8. PVPALLNSSTTEQFLNTVAQ (SEQ ID NO: 8)
    9. LNSSTTEQFLNTVAQLPTDL (SEQ ID NO: 9)
  10. TEQFLNTVAQLPTDLSKFIR (SEQ ID NO: 10)
  11. NTVAQLPTDLSKFIRDYRVF (SEQ ID NO: 11)
  12. LPTDLSKFIRDYRVFALVRA (SEQ ID NO: 12)
  13. SKFIRDYRVFALVRAAYFLE (SEQ ID NO: 13)
  14. DYRVFALVRAAYFLEPPSSI (SEQ ID NO: 14)
  15. ALVRAAYFLEPPSSIDPLEA (SEQ ID NO: 15)
  16. AYFLEPPSSIDPLEAARALG (SEQ ID NO: 16)
  17. PPSSIDPLEAARALGRLVDI (SEQ ID NO: 17)
  18. DPLEAARALGRLVDILSSQP (SEQ ID NO: 18)
  19. ARALGRLVDILSSQPPQNTA (SEQ ID NO: 19)
  20. RLVDILSSQPPQNTAPAQPP (SEQ ID NO: 20)
  21. LSSQPPQNTAPAQPPTSDDT (SEQ ID NO: 21)
  22. PQNTAPAQPPTSDDTLNNCT (SEQ ID NO: 22)
  23. PAQPPTSDDTLNNCTLLKLL (SEQ ID NO: 23)
  24. TSDDTLNNCTLLKLLAHYAD (SEQ ID NO: 24)
  25. LNNCTLLKLLAHYADQIAGF (SEQ ID NO: 25)
  26. LLKLLAHYADQIAGFKTPAL (SEQ ID NO: 26)
  27. AHYADQIAGFKTPALPPVPP (SEQ ID NO: 27)
  28. QIAGFKTPALPPVPPGIIGL (SEQ ID NO: 28)
  29. KTPALPPVPPGIIGLFTCVE (SEQ ID NO: 29)
  30. PPVPPGIIGLFTCVEQMYHA (SEQ ID NO: 30)
  31. GIIGLFTCVEQMYHACFQKY (SEQ ID NO: 31)
  32. FTCVEQMYHACFQKYWAAAL (SEQ ID NO: 32)
  33. QMYHACFQKYWAAALPPMWI (SEQ ID NO: 33)
  34. CFQKYWAAALPPMWILTYDP (SEQ ID NO: 34)
  35. WAAALPPMWILTYDPPTSPL (SEQ ID NO: 35)
  36. PPMWILTYDPPTSPLQDWLI (SEQ ID NO: 36)
  37. LTYDPPTSPLQDWLIVAYGN (SEQ ID NO: 37)
  38. PTSPLQDWLIVAYGNKEGLL (SEQ ID NO: 38)
  39. QDWLIVAYGNKEGLLLPSGI (SEQ ID NO: 39)
  40. VAYGNKEGLLLPSGIPSEEV (SEQ ID NO: 40)
  41. KEGLLLPSGIPSEEVLAKTL (SEQ ID NO: 41)
  42. LPSGIPSEEVLAKTLVTEHH (SEQ ID NO: 42)
  43. PSEEVLAKTLVTEHHELFVS (SEQ ID NO: 43)
  44. LAKTLVTEHHELFVSRSNST (SEQ ID NO: 44)
  45. VTEHHELFVSRSNSTETAVT (SEQ ID NO: 45)
  46. ELFVSRSNSTETAVTMPVSK (SEQ ID NO: 46)
  47. RSNSTETAVTMPVSKERALA (SEQ ID NO: 47)
  48. ETAVTMPVSKERALAIYRVF (SEQ ID NO: 48)
  49. MPVSKERALAIYRVFAKGEV (SEQ ID NO: 49)
  50. ERALAIYRVFAKGEVVAENT (SEQ ID NO: 50)
  51. IYRVFAKGEVVAENTPILAF (SEQ ID NO: 51)
  52. AKGEVVAENTPILAFTDVEL (SEQ ID NO: 52)
  53. VAENTPILAFTDVELSTLKP (SEQ ID NO: 53)
  54. PILAFTDVELSTLKPHYLFI (SEQ ID NO: 54)
  55. TDVELSTLKPHYLFIYDFII (SEQ ID NO: 55)
  56. STLKPHYLFIYDFIIEALCK (SEQ ID NO: 56)
  57. HYLFIYDFIIEALCKSYTYS (SEQ ID NO: 57)
  58. YDFIIEALCKSYTYSCTQAR (SEQ ID NO: 58)
  59. EALCKSYTYSCTQARLESFL (SEQ ID NO: 59)
  60. SYTYSCTQARLESFLSRGID (SEQ ID NO: 60)
  61. CTQARLESFLSRGIDFMTDL (SEQ ID NO: 61)
  62. LESFLSRGIDFMTDLGQYLD (SEQ ID NO: 62)
  63. SRGIDFMTDLGQYLDTATSG (SEQ ID NO: 63)
  64. FMTDLGQYLDTATSGKQQLT (SEQ ID NO: 64)
  65. GQYLDTATSGKQQLTHSQIK (SEQ ID NO: 65)
  66. TATSGKQQLTHSQIKEIKYR (SEQ ID NO: 66)
  67. KQQLTHSQIKEIKYRLLSCG (SEQ ID NO: 67)
  68. HSQIKEIKYRLLSCGLSASA (SEQ ID NO: 68)
  69. EIKYRLLSCGLSASACDVFR (SEQ ID NO: 69)
  70. LLSCGLSASACDVFRTVIMT (SEQ ID NO: 70)
  71. LSASACDVFRTVIMTLPYRP (SEQ ID NO: 71)
  72. CDVFRTVIMTLPYRPTPNLA (SEQ ID NO: 72)
  73. TVIMTLPYRPTPNLANLSTF (SEQ ID NO: 73)
  74. LPYRPTPNLANLSTFMGMVH (SEQ ID NO: 74)
  75. TPNLANLSTFMGMVHQLTMF (SEQ ID NO: 75)
  76. NLSTFMGMVHQLTMFGHYFY (SEQ ID NO: 76)
  77. MGMVHQLTMFGHYFYRCLGS (SEQ ID NO: 77)
  78. QLTMFGHYFYRCLGSYSPTG (SEQ ID NO: 78)
  79. GHYFYRCLGSYSPTGLAFTE (SEQ ID NO: 79)
  80. RCLGSYSPTGLAFTELQKIL (SEQ ID NO: 80)
  81. YSPTGLAFTELQKILTRASA (SEQ ID NO: 81)
  82. LAFTELQKILTRASAEQTER (SEQ ID NO: 82)
  83. LQKILTRASAEQTERNPWRH (SEQ ID NO: 83)
  84. TRASAEQTERNPWRHPGISD (SEQ ID NO: 84)
  85. EQTERNPWRHPGISDIPLRW (SEQ ID NO: 85)
  86. NPWRHPGISDIPLRWKISRA (SEQ ID NO: 86)
  87. PGISDIPLRWKISRALAFFV (SEQ ID NO: 87)
  88. IPLRWKISRALAFFVPPAPI (SEQ ID NO: 88)
  89. KISRALAFFVPPAPINTLQR (SEQ ID NO: 89)
  90. LAFFVPPAPINTLQRVYAAL (SEQ ID NO: 90)
  91. PPAPINTLQRVYAALPSQLM (SEQ ID NO: 91)
  92. NTLQRVYAALPSQLMRAIFE (SEQ ID NO: 92)
  93. VYAALPSQLMRAIFEISVKT (SEQ ID NO: 93)
  94. PSQLMRAIFEISVKTTWGGA (SEQ ID NO: 94)
  95. RAIFEISVKTTWGGAVPANL (SEQ ID NO: 95)
  96. ISVKTTWGGAVPANLARDID (SEQ ID NO: 96)
  97. TWGGAVPANLARDIDTGPNT (SEQ ID NO: 97)
  98. VPANLARDIDTGPNTQHISS (SEQ ID NO: 98)
  99. ARDIDTGPNTQHISSTPPPT (SEQ ID NO: 99)
100. TGPNTQHISSTPPPTLKDVE (SEQ ID NO: 100)
101. QHISSTPPPTLKDVETYCQG (SEQ ID NO: 101)
102. TPPPTLKDVETYCQGLRVGD (SEQ ID NO: 102)
103. LKDVETYCQGLRVGDTEYDE (SEQ ID NO: 103)
104. TYCQGLRVGDTEYDEDIVRS (SEQ ID NO: 104)
105. LRVGDTEYDEDIVRSPLFAD (SEQ ID NO: 105)
106. TEYDEDIVRSPLFADAFTKS (SEQ ID NO: 106)
107. DIVRSPLFADAFTKSHLLPI (SEQ ID NO: 107)
108. PLFADAFTKSHLLPILREVL (SEQ ID NO: 108)
109. AFTKSHLLPILREVLENRLQ (SEQ ID NO: 109)
110. HLLPILREVLENRLQKNRAL (SEQ ID NO: 110)
111. LREVLENRLQKNRALFQIRW (SEQ ID NO: 111)
112. ENRLQKNRALFQIRWLIIFA (SEQ ID NO: 112)
113. KNRALFQIRWLIIFAAEAAT (SEQ ID NO: 113)
114. FQIRWLIIFAAEAATGLIPA (SEQ ID NO: 114)
115. LIIFAAEAATGLIPARRPLA (SEQ ID NO: 115)
116. AEAATGLIPARRPLARAYFH (SEQ ID NO: 116)
117. GLIPARRPLARAYFHIMDIL (SEQ ID NO: 117)
118. RRPLARAYFHIMDILEERHS (SEQ ID NO: 118)
119. RAYFHIMDILEERHSQDALY (SEQ ID NO: 119)
120. IMDILEERHSQDALYNLLDC (SEQ ID NO: 120)
121. EERHSQDALYNLLDCIQELF (SEQ ID NO: 121)
122. QDALYNLLDCIQELFTHIRQ (SEQ ID NO: 122)
123. NLLDCIQELFTHIRQAVPDA (SEQ ID NO: 123)
124. IQELFTHIRQAVPDAQCPHA (SEQ ID NO: 124)
125. THIRQAVPDAQCPHAFLQSL (SEQ ID NO: 125)
126. AVPDAQCPHAFLQSLFVFQF (SEQ ID NO: 126)
127. QCPHAFLQSLFVFQFRPFVL (SEQ ID NO: 127)
128. FLQSLFVFQFRPFVLKHQQG (SEQ ID NO: 128)
129. FVFQFRPFVLKHQQGVTLFL (SEQ ID NO: 129)
130. RPFVLKHQQGVTLFLDGLQT (SEQ ID NO: 130)
131. KHQQGVTLFLDGLQTSLPPV (SEQ ID NO: 131)
132. VTLFLDGLQTSLPPVISLAN (SEQ ID NO: 132)
133. DGLQTSLPPVISLANLGDKL (SEQ ID NO: 133)
134. SLPPVISLANLGDKLCRLEF (SEQ ID NO: 134)
135. ISLANLGDKLCRLEFEYDSE (SEQ ID NO: 135)
136. LGDKLCRLEFEYDSEGDFVR (SEQ ID NO: 136)
137. CRLEFEYDSEGDFVRVPVAP (SEQ ID NO: 137)
138. EYDSEGDFVRVPVAPPEQPP (SEQ ID NO: 138)
139. GDFVRVPVAPPEQPPHVHLS (SEQ ID NO: 139)
140. VPVAPPEQPPHVHLSHFKKT (SEQ ID NO: 140)
141. PEQPPHVHLSHFKKTIQTIE (SEQ ID NO: 141)
142. HVHLSHFKKTIQTIEQATRE (SEQ ID NO: 142)
143. HFKKTIQTIEQATREATVAM (SEQ ID NO: 143)
144. IQTIEQATREATVAMTTIAK (SEQ ID NO: 144)
145. QATREATVAMTTIAKPIYPA (SEQ ID NO: 145)
146. ATVAMTTIAKPIYPAYIRLL (SEQ ID NO: 146)
147. TTIAKPIYPAYIRLLQRLEY (SEQ ID NO: 147)
148. PIYPAYIRLLQRLEYLNRLN (SEQ ID NO: 148)
149. YIRLLQRLEYLNRLNHHILR (SEQ ID NO: 149)
150. QRLEYLNRLNHHILRIPFPQ (SEQ ID NO: 150)
151. LNRLNHHILRIPFPQDALSE (SEQ ID NO: 151)
152. HHILRIPFPQDALSELQETY (SEQ ID NO: 152)
153. IPFPQDALSELQETYLAAFA (SEQ ID NO: 153)
154. DALSELQETYLAAFARLTKL (SEQ ID NO: 154)
155. LQETYLAAFARLTKLAADAA (SEQ ID NO: 155)
156. LAAFARLTKLAADAANTCSY (SEQ ID NO: 156)
157. RLTKLAADAANTCSYSLTKY (SEQ ID NO: 157)
158. AADAANTCSYSLTKYFGVLF (SEQ ID NO: 158)
159. AADAANTCSYSLTKYFGVLF (SEQ ID NO: 159)
160. SLTKYFGVLFQHQLVPTAIV (SEQ ID NO: 160)
161. FGVLFQHQLVPTAIVKKLLH (SEQ ID NO: 161)
162. QHQLVPTAIVKKLLHFDEAK (SEQ ID NO: 162)
163. PTAIVKKLLHFDEAKDTTEA (SEQ ID NO: 163)
164. KKLLHFDEAKDTTEAFLQSL (SEQ ID NO: 164)
165. FDEAKDTTEAFLQSLAQPVV (SEQ ID NO: 165)
166. DTTEAFLQSLAQPVVQGQRQ (SEQ ID NO: 166)
167. FLQSLAQPVVQGQRQGAAGG (SEQ ID NO: 167)
168. AQPVVQGQRQGAAGGSGVLT (SEQ ID NO: 168)
169. QGQRQGAAGGSGVLTQGELE (SEQ ID NO: 169)
170. GAAGGSGVLTQKELELLNKI (SEQ ID NO: 170)
171. SGVLTQKELELLNKINPQFT (SEQ ID NO: 171)
172. QKELELLNKINPQFTDAQAN (SEQ ID NO: 172)
173. LLNKINPQFTDAQANIPPSI (SEQ ID NO: 173)
174. NPQFTDAQANIPPSIKRSYS (SEQ ID NO: 174)
175. DAQANIPPSIKRSYSNKYDV (SEQ ID NO: 175)
176. IPPSIKRSYSNKYDVPEVSV (SEQ ID NO: 176)
177. KRSYSNKYDVPEVSVDWETY (SEQ ID NO: 177)
178. NKYDVPEVSVDWETYSRSAF (SEQ ID NO: 178)
179. PEVSVDWETYSRSAFEAPDD (SEQ ID NO: 179)
180. DWETYSRSAFEAPDDELRFV (SEQ ID NO: 180)
181. SRSAFEAPDDELRFVPLTLA (SEQ ID NO: 181)
182. EAPDDELRFVPLTLAGLRKL (SEQ ID NO: 182)
183. ELRFVPLTLAGLRKLFVE (SEQ ID NO: 183)
```

1. A method of reducing inflammation in a subject, comprising administering to the subject an effective amount of a KSHV Orf63 protein or a biologically active fragment thereof and/or a KSHV Orf63 peptide, thereby reducing inflammation in the subject.

2. A method of treating an inflammatory disorder in a subject in need thereof, comprising administering to the subject an effective amount of a KSHV Orf63 protein or a biologically active fragment thereof and/or a KSHV Orf63 peptide, thereby treating the inflammatory disorder in the subject.

3. A method of inhibiting activity in a subject of a proinflammatory molecule selected from the group consisting of interleukin-1β, interleukin-18, NLRP1, NLRP3, Caspase-1, any other NLR protein family member and any combination thereof, comprising administering to the subject an effective amount of a KSHV Orf63 protein or a biologically active fragment thereof and/or a KSHV Orf63 peptide, thereby inhibiting activity of the proinflammatory molecule in the subject.

4. The method of claim 1, wherein the KSHV Orf63 protein or biologically active fragment thereof and/or peptide is administered directly to a site of inflammation in the subject.

5. The method of claim 1, wherein the KSHV Orf63 protein or biologically active fragment thereof and/or peptide is administered to the subject intravenously, orally and/or transdermally.

6. The method of claim 1, wherein a KSHV Orf63 peptide is administered to the subject and the peptide is selected from the group consisting of:
a) a peptide having the amino acid sequence of SEQ ID NO:45;
b) a peptide having the amino acid sequence of SEQ ID NO:46;
c) a peptide having the amino acid sequence of SEQ ID NO:47;
d) a peptide having the amino acid sequence of SEQ ID NO:48;
e) a peptide having the amino acid sequence of SEQ ID NO:49;
f) a peptide having the amino acid sequence of SEQ ID NO:50;
g) a peptide having the amino acid sequence of SEQ ID NO:51;
h) a peptide having the amino acid sequence of SEQ ID NO:52;
i) a peptide having the amino acid sequence of SEQ ID NO:53;
j) a peptide having the amino acid sequence of SEQ ID NO:54;
k) a peptide having the amino acid sequence of SEQ ID NO:55;
l) a peptide having the amino acid sequence of SEQ ID NO:56;
m) a peptide having the amino acid sequence of SEQ ID NO:57;
n) a peptide having the amino acid sequence of SEQ ID NO:58;
o) a peptide having the amino acid sequence of SEQ ID NO:59;
p) a peptide having the amino acid sequence of SEQ ID NO:60;
q) a peptide having the amino acid sequence of SEQ ID NO:61;
r) a peptide having the amino acid sequence of SEQ ID NO:62;
s) a peptide having the amino acid sequence of SEQ ID NO:63;
t) a peptide having the amino acid sequence of SEQ ID NO:64;
u) a peptide having the amino acid sequence of SEQ ID NO:65;
v) a peptide having the amino acid sequence of SEQ ID NO:66;
w) a peptide having the amino acid sequence of SEQ ID NO:67;
x) a peptide having the amino acid sequence of SEQ ID NO:68;
y) a peptide having the amino acid sequence of SEQ ID NO:69;
z) a peptide having the amino acid sequence of SEQ ID NO:70;
aa) a peptide having the amino acid sequence of SEQ ID NO:71; and
bb) any combination of (a) through (aa) above.

7. The method of claim 1, wherein the effective amount of the KSHV Orf63 protein or biologically active fragment thereof or the KSHV Orf63 peptide is in the range of about 1 microgram/ml to about 500 milligrams/ml.

8. The method of claim 1, further comprising administering an anti-inflammatory agent, an anti-microbial agent, a tissue regeneration agent or any combination thereof to the subject.

9. The method of claim 8, wherein the anti-inflammatory agent is selected from the group consisting of an inhibitor of interleukin-1 (IL-1), an inhibitor of interleukin-6 (IL-6), an inhibitor of tumor necrosis factor alpha (TNF-α), an inhibitor of Caspase-1, an inhibitor of NF-KB or members of the NF-KB pathway that activate NF-KB, an inhibitor of matrix metalloproteinase (MMP) 1, 2, 8 and/or 9, an inhibitor of p38 mitogen activated protein kinase (MAPK), an inhibitor of extracellular signal-related kinase (ERK) (ERK1; ERK2), SBR203580 (p38 inhibitor), PD98059 (ERK inhibitor), U0126 (inhibitor of MMP expression) simvastatin (inhibitor of MMP-1 expression), and any combination thereof.

10. The method of claim 2, wherein the inflammatory disorder is selected from the group consisting of atherosclerosis, ulcerative colitis, inflammatory bowel disease, Crohn's disease, pancreatitis, pelvic inflammatory disease, rheumatoid arthritis, osteoarthritis, asthma, hay fever, seasonal allergies, perennial allergies, vasculitis, psoriasis, allergic rhinitis, peptic ulcer disease, acne vulgaris, dermatitis, hypersensitivity, glomerulonephritis, sarcoidosis, inflammation-associated cancer, transplant rejection, gout, postoperative intra-abdominal sepsis, ischemia-reperfusion injury, pancreatic damage, liver damage, sepsis, septic shock, gastric damage caused by certain drugs, stress-induced gastric damage, gastric damage caused by H. pylori, inflammatory pain, chronic kidney disease, intestinal inflammation, autoimmune disorder, familial cold autoinflammatory syndrome, Muckle Wells syndrome, neonatal onset multisystem inflammatory disease (NOMID), vitiligo, and any combination thereof.

11. The method of claim 1, wherein the inflammation is caused by infection, trauma, allergic reaction, surgical wounding, autoimmune dysfunction, autoinflammatory disorder, autoimmune disorder, genetic predisposition, allergic reaction, surgical wounding, burn, tissue damage, tissue incision and any combination thereof.

12. An isolated peptide comprising the amino acid sequence of any of SEQ ID NOs:1-183 or any combination thereof.

13. The isolated peptide of claim 12, comprising:
a) the amino acid sequence of SEQ ID NO:45;
b) the amino acid sequence of SEQ ID NO:46;
c) the amino acid sequence of SEQ ID NO:47;
d) the amino acid sequence of SEQ ID NO:48;
e) the amino acid sequence of SEQ ID NO:49;
f) the amino acid sequence of SEQ ID NO:50;
g) the amino acid sequence of SEQ ID NO:51;
h) the amino acid sequence of SEQ ID NO:52;
i) the amino acid sequence of SEQ ID NO:53;
j) the amino acid sequence of SEQ ID NO:54;
k) the amino acid sequence of SEQ ID NO:55;
l) the amino acid sequence of SEQ ID NO:56;
m) the amino acid sequence of SEQ ID NO:57;
n) the amino acid sequence of SEQ ID NO:58;
o) the amino acid sequence of SEQ ID NO:59;
p) the amino acid sequence of SEQ ID NO:60;
q) the amino acid sequence of SEQ ID NO:61;
r) the amino acid sequence of SEQ ID NO:62;
s) the amino acid sequence of SEQ ID NO:63;
t) the amino acid sequence of SEQ ID NO:64;
u) the amino acid sequence of SEQ ID NO:65;
v) the amino acid sequence of SEQ ID NO:66;
w) the amino acid sequence of SEQ ID NO:67;
x) the amino acid sequence of SEQ ID NO:68;
y) the amino acid sequence of SEQ ID NO:69;
z) the amino acid sequence of SEQ ID NO:70;
aa) the amino acid sequence of SEQ ID NO:71; and
bb) any combination of (a) through (aa) above.

14. A composition comprising the isolated peptide or combination thereof of claim 12 in a pharmaceutically acceptable carrier.

15. The composition of claim 14, further comprising a KSHV Orf63 protein.

16. An isolated nucleic acid encoding a biologically active fragment or peptide of a KSHV Orf63 protein.

17. A virus particle comprising the nucleic acid of claim 16.

18. A composition comprising the isolated nucleic acid of claim 16, in a pharmaceutically acceptable carrier.

19. The composition of claim 14, further comprising an anti-inflammatory agent, an antimicrobial agent, a tissue regeneration agent or any combination thereof.

20. A composition comprising the virus particle of claim 17.
